(19) United States
(12) Patent Application Publication
(10) Pub. No.: US 2005/0255504 A1
(43) Pub. Date: Nov. 17, 2005

(54) Method of detecting an increased susceptibility to breast cancer

(76) Inventor: Fritz F. Parl, Nashville, TN (US)

Correspondence Address: NEEDLE & ROSENBERG, P.C., SUITE 1000, 999 PEACHTREE STREET, ATLANTA, GA, 30309-3915, US

(21) Appl. No.: 11/052,544

(22) Filed: Feb. 7, 2005

Related U.S. Application Data
(60) Provisional application No. 60/543,866, filed on Feb. 12, 2004.

(51) Int. Cl. C12Q 1/68

(52) U.S. Cl. 435/6

(57) ABSTRACT

The present invention provides methods for identifying a subject having an increased risk of developing cancer, for example, breast cancer, comprising determining the presence of the homozygous wild-type genotype of GSTM1, wherein the presence of the homozygous wild-type genotype of GSTM1 identifies a subject with increased risk of cancer. The present invention also provides methods for identifying a subject having a reduced risk of cancer, for example, breast cancer, comprising determining the presence of the homozygous null allele genotype of GSTM1, wherein the presence of the homozygous null allele genotype of GSTM1 identifies a subject with decreased risk of cancer. Also provided are isolated nucleotide sequences and kits for identifying the GSTM1 genotype in a subject.

This application claims priority to U.S. provisional application No. 60/543,866, filed Feb. 12, 2004. The aforementioned application is herein incorporated by this reference in its entirety.

This invention was made with government support under NIH Grants CA/ES83752, CA50468, P50-CA98131 and U.S. Army Breast Cancer Training Grant DAMD-17-94-J4024. The government has certain rights in the invention.

### BACKGROUND OF THE INVENTION

1. Field of the Invention

This invention relates generally to a method of detecting an increased susceptibility to cancer. In particular, the present invention provides a method of detecting an increased risk of developing breast cancer.

2. Background Art

Glutathione S-transferases (GSTs) constitute a superfamily of ubiquitous, multifunctional enzymes, which play a key role in cellular detoxification (139). The GSTs catalyze the conjugation of the tripeptide glutathione (GSH) to a wide variety of exogenous and endogenous chemicals with electrophilic functional groups (e.g., products of oxidative stress, environmental pollutants, and carcinogens), thereby neutralizing their electrophilic sites, and rendering the products more water-soluble (56). Based on sequence homology and immunological crossreactivity, human cytosolic GSTs have been grouped into seven families, designated GST Alpha, Mu, Pi, Sigma, Omega, Theta, and Zeta (39, 94). The GSTm subfamily is encoded by a 100-kb gene cluster at 1p13.3 arranged as 5′-GSTM4-GSTM2-GSTM1-GSTM5-GSTM3-3′ (156). Deletion of the GSTM1 gene, GSTM1-0 frequently affects both alleles, resulting in the so-called null genotype, GSTM1−/−. A meta-analysis of 30 studies involving over 10,000 individuals identified the GSTM1 null genotype in 53% Caucasians, with a 42 to 60% range for individual studies (8, 43). The frequency of the GSTM1 null genotype was similar in Asians but lower in African-Americans, 27% (16-36%). Detailed mapping of the GSTα gene cluster revealed that the GSTM1 gene is flanked by two almost identical 4.2-kb regions. The GSTM1-0 deletion is caused by homologous recombination involving the left and right 4.2-kb repeats (156). Analysis of 20 GSTM1-0 alleles from 13 unrelated individuals showed the same recombination pattern which results in a 16-kb deletion containing the entire GSTM1 gene. The GSTM1 gene is excised relatively precisely leaving the adjacent GSTM2 and GSTM5 genes intact. Therefore, one can rule out recombination with neighboring GSTM genes as a possible mechanism for the GSTM1-0 deletion, despite extensive homologies in certain regions.

In view of the importance of GSTs in cellular detoxification, the enzyme deficiency associated with the GSTM1 null genotype has attracted considerable attention with regard to cancer epidemiology. A search of the literature published from 1993 to 2003 listed over 500 studies of the GSTM1 genotype in relation to lung, breast, colon, brain, and various other types of cancer. These studies have in common PCR-based genotyping using an assay designed to identify the wild-type (wt) allele of GSTM1 (117). The absence of a PCR product (273 bp) indicates the GSTM1 null genotype. Consequently, study participants were categorized as either wt or null ‘genotypes’. This analytical approach has one basic flaw in that it does not positively identify the null allele and, therefore, cannot distinguish homozygous wt/wt from heterozygous wt/− individuals. Assuming that the presence of 2, 1, or 0 GSTM1 alleles is associated with a gene dosage effect resulting in high-, low-, or non-GSTM1 conjugator phenotypes, the current approach oversimplifies phenotypes as all or none. Not surprisingly, the large number of studies utilizing this approach has yielded confusing data, which resulted in inconsistent or contradictory publications on the association of the GSTM1 ‘genotype’ with various malignancies (36, 44, 130).

Estrogens are clearly carcinogenic in humans and rodents but the molecular pathways by which these hormones induce cancer are only partially understood. In broad terms, two distinct mechanisms of estrogen carcinogenicity have been outlined. Stimulation of cell proliferation and gene expression by binding to the estrogen receptor is one important mechanism in hormonal carcinogenesis (Nandi, 1995). However, estrogenicity is not sufficient to explain the carcinogenic activity of all estrogens because some estrogens are not carcinogenic. Increasing evidence of a second mechanism of carcinogenicity has focused attention on catechol estrogen metabolites, which are less potent estrogens than 17β-estradiol (E2), but can directly or indirectly induce various types of DNA damage ranging from modification of bases to single-strand breakage, all of which are thought to have mutagenic potential (Cavalieri, 1997; Floyd, 1990; Han, 1994; Yager, 1996).

The two main estrogens, E2 and estrone (E1), are metabolized to catechol estrogens, their 2-OH and 4-OH derivatives. Two phase I enzymes, CYP1A1 and CYP1B1, are responsible for the hydroxylation of E2 and E1 to the 2-OH and 4-OH catechol estrogens (i.e., 2-OHE1, 2OHE2,4-OHE1, and 4-OHE2;). The 2-OH and 4-OH catechol estrogens are oxidized to semiquinones (E1-2,3SQ, E2-2,3SQ, E1-3,4SQ, and E2-3,4SQ) and quinones (E1-2,3Q, E2-2,3Q, E1-3,4Q, and E2-3,4Q). The latter are highly reactive electrophilic metabolites that are capable of forming DNA adducts (Abul-Hajj, 1988; Dwivedy, 1992). Further DNA damage results from quinone-semiquinone redox cycling, generated by enzymatic reduction of catechol estrogen quinones to semiquinones and subsequent autoxidation back to quinones (Liehr, 1986; Liehr, 1990; Liehr, 1990). Two phase II enzymes, i.e., catechol-O-methyltransferase (COMT) and glutathione S-transferases (GSTs), either inactivate catechol estrogens or protect against estrogen carcinogenesis by detoxifying products of oxidative damage that may arise upon redox cycling of catechol estrogens. COMT inactivates 2-OH and 4-OH catechol estrogens by O-methylation, forming 2-MeO and 4-MeO methoxy estrogens (Roy, 1990). GSTP1, and GSTT1 inactivate catechol estrogen quinones by conjugation with glutathione (Iverson, 1996).

Although other cytochrome P450 enzymes, such as CYP1A2 and CYP3A4, are involved in hepatic and extrahepatic estrogen hydroxylation, CYP1A1 and CYP1B1 display the highest level of expression in breast tissue (Huang, 1997; Shimada, 1996). In turn, CYP1B1 exceeds CYP1A1 in its catalytic efficiency as E2 hydroxylase and differs from CYP1A1 in its principal site of action (Hayes, 1996; Spink, 1992; Spink, 1994). CYP1B1 has its primary activity at the C-4 position of E2, whereas CYP1A1 has its primary activity at the C-2 position in preference to 4-hydroxylation. Thus, CYP1B1 appears to be the main cytochrome P450 responsible for the 4-hydroxylation of E2. The 4-hydroxylation activity of CYP1B1 has received particular attention due to the fact that the 2-OH and 4-OH catechol estrogens differ in carcinogenicity. Treatment with 4-OHE2 and 4-OHE 1, but not 2-OHE2 and 2-OHE1, induced renal cancer in Syrian hamster (Li, 1987; Liehr, 1986). Analysis of renal DNA demonstrated that 4-OHE2 and 4-OHE1 significantly increased levels of the oxidized base 8-hydroxy-deoxyguanosine, while 2-OHE2 did not cause oxidative DNA damage (Han, 1995). Similarly, 4-OHE2 induced DNA single-strand breaks while 2-OHE2 had a negligible effect. Comparison of the corresponding catechol estrogen quinones showed that E2-3,4Q and E1-3,4Q produced two to three orders of magnitude higher levels of depurinating DNA adducts than E2-2,3Q and E1-2,3Q (Cavalieri, 1997). Finally, examination of microsomal E2 hydroxylation in human breast cancer showed significantly higher 4-OHE2/2-OHE2 ratios in tumor tissue than in adjacent normal breast tissue (Liehr, 1996). All these findings support a causative role of 4-OH catechol estrogens in carcinogenesis and implicate CYP1B1 as a key player in the process.

Genetic variants of each of the enzymes involved in catechol estrogen metabolism have been identified. The CYP1A1 gene possesses four polymorphisms of which two result in amino acid substitutions: codon 461Thr→Asn and codon 462Ile→Val (Cascorbi, 1996; Hayashi, 1991). Six polymorphisms of the CYP1B1 gene have been described, of which four result in amino acid substitutions (Bailey, 1998; Stoilov, 1998). Two of these amino acid substitutions: codon 432Val→Leu and codon 453Asn→Ser) have been described (Bailey, 1998). Stoilov et al. (Stoilov, 1998) described the other two amino acid substitutions in codons 48 (Arg→Gly) and 119 (Ala→Ser). The COMT gene possesses a common polymorphism in codon 158Val→Met (Lachman, 1996). The GSTP1 gene contains polymorphisms in codons 105Ile→Val and 113Ala→Val (Ali-Osman, 1997; Zimniak, 1994). The functional implications of these polymorphisms in terms of enzyme activities have been investigated. The 462Ile→Val substitution in recombinant variant CYP1A1 does not appear to alter enzymatic activity (Persson, 1997; Zhang, 1996). However, in vivo CYP1A1 activity was more readily inducible in lymphocytes with the Val/Val genotype than in wild type lymphocytes (Cosma, 1993). Recombinant wild type and each of the polymorphic variants of CYP1B1 were expressed and purified, followed by assays of E2 hydroxylation activity (Hanna, 2000). Quantitation of 2-OH-E2 and 4-OH-E2 by gas chromatography/mass spectrometry showed that the CYP1B1 variants displayed 2.4- to 3.4-fold higher catalytic efficiencies than the wild type enzyme. Using catecholamines as substrate, Syvanen et al. (Syvanen, 1997) determined that COMT activity in red blood cells from individuals with the homozygous Met/Met genotype was reduced two-thirds compared to individuals with the homozygous Val/Val wild type. Heterozygotes showed intermediate activity. It is likely that the polymorphism in codon 158Val→Met affects O-methylation of catechol estrogens in a similar manner because both catecholamines and catechol estrogens are recognized as catechol substrates by COMT. The GSTP1 polymorphisms in codons 105Ile→Val and 113Ala→Val are associated with a 3- to 4-fold reduction in catalytic activity compared to wild type GSTP1 (Ali-Osman, 1997; Zimniak, 1994). Approximately 20% of individuals possess the homozygous null GSTT1 genotype and are therefore devoid of functional GSTT1 enzyme (Wiencke, 1995). Thus, inherited alterations in the activity of any of these six enzymes may be associated with significant changes in estrogen metabolism. The associated interindividual differences in life-long exposure to carcinogenic catechol estrogens hold the potential to explain differences in breast cancer risk.

The present invention shows that inherited alterations in CYP1A1, CYP1B1, COMT, GSTP1, and GSTT1 activity are useful in predicting increased risk of developing an estrogen-related cancer, such as breast cancer.

### SUMMARY OF THE INVENTION

The present invention provides a method for identifying a subject having an increased risk of developing cancer, comprising determining the allele or alleles of the subject's GSTM1 gene, whereby a subject being homozygous for the wild-type allele or a subject being heterozygous for the wild-type and null alleles is identified as having an increased risk of developing cancer, for example, breast cancer.

The present invention further provides a method for identifying a subject having a decreased risk of developing cancer, comprising determining the allele or alleles of the subject's GSTM1 gene, whereby a subject having an allele of the GSTM1 gene which is correlated with a decreased risk of developing cancer and which comprises a homozygous null allele is identified as having a decreased risk of developing cancer, for example, breast cancer.

Another aspect of the present invention is a diagnostic kit for determining the presence in a subject of an allele of the gene encoding GSTM1 that is correlated with an increased risk of developing cancer, comprising means for distinguishing a homozygous wild-type subject or a heterozygous wild-type/null subject from a subject with a homozygous null genotype.

The kit comprises an identifying means comprising a first nucleic acid primer pair having a nucleic acid with the sequence identified as SEQ ID NO:20 and a nucleic acid with the sequence identified as SEQ ID NO:21, and a second nucleic acid primer pair selected from the group of a nucleic acid primer pair having a nucleic acid with the sequence identified as SEQ ID NO:22 and a nucleic acid with the sequence identified as SEQ ID NO:23; a nucleic acid primer pair having a nucleic acid with the sequence identified as SEQ ID NO:24 and a nucleic acid with the sequence identified as SEQ ID NO:25; and a nucleic acid primer pair having a nucleic acid with the sequence identified as SEQ ID NO:26 and a nucleic acid with the sequence identified as SEQ ID NO:27.

The present invention further provides a diagnostic kit for determining the presence in a subject of a homozygous null allele of the gene encoding GSTM1 that is correlated with a decreased risk of developing cancer, comprising means for identifying the allele of the subject's GSTM1 gene in a biological sample from the subject, wherein the identifying means comprises a nucleic acid primer pair selected from the group of a nucleic acid primer pair having a nucleic acid with the sequence identified as SEQ ID NO:3 and a nucleic acid with the sequence identified as SEQ ID NO:23; a nucleic acid primer pair having a nucleic acid with the sequence identified as SEQ ID NO:24 and a nucleic acid with the sequence identified as SEQ ID NO:25; and a nucleic acid primer pair having a nucleic acid with the sequence identified as SEQ ID NO:26 and a nucleic acid with the sequence identified as SEQ ID NO:27.

The present invention also provides isolated nucleic acids that can be used, according to methods well-known in the art, to identify a subject with the homozygous wild-type genotype of GSTM1, the heterozygous wild-type/null genotype of GSTM1 and the homozygous null-type genotype of GSTM1, for example, SEQ ID NO:20, SEQ ID NO:21, SEQ ID NO:22, SEQ ID NO:23, SEQ ID NO:24, SEQ ID NO:25, SEQ ID NO:26 and SEQ ID NO:27.

The invention provides primers, wherein the primers are from about 15 to about 35 nucleotides in length, and wherein a primer has a nucleotide sequence specific for cosmid clone cgtm1 from about nucleotide 15734 to about nucleotide 17595 or a nucleotide sequence specific for cosmid clone cgtm12 from about nucleotide 8402 to about nucleotide 10260.

The present invention also provides primer pairs that can be used with various methods known in the art, for example PCR, to identify a subject as being homozygous for the wild-type allele of GSTM1, or heterozygous for the wild-type allele and the null allele of GSTM1, or homozygous for the null allele of GSTM1. Examples of these primer pairs include, but are not limited to, nucleic acids with the sequences identified as SEQ ID NO:20 and SEQ ID NO:21; SEQ ID NO:22 and SEQ ID NO:23; SEQ ID NO:24 and SEQ ID NO:25; and SEQ ID NO:26, and SEQ ID NO:27.

The present invention provides a method for identifying a subject having an increased risk of developing an estrogen-related cancer comprising determining which alleles of the genes encoding. CYP 1 B 1 and COMT are present in the genome of the subject so as to determine an estrogen metabolizing enzyme genotype for the individual, and correlating the estrogen metabolizing enzyme genotype of the individual to an increased risk of developing breast cancer, wherein a subject having an estrogen metabolizing enzyme genotype comprising one of
(a) CYP1B1 432Val/Leu, CYP1B1 453Asn/Ser,
(b) CYP1B1 432Val/Leu, CYP1B1 453Ser/Ser,
(c) CYP1B1 432Val/Leu, COMT 158Val/Met,
(d) CYP1B1 432Val/Leu, COMT 158Met/Met;
(e) CYP1B1 432Val/Val, CYP1B1 453Asn/Ser,
(f) CYP1B1 432Val/Val, CYP1B1 453Ser/Ser,
(g) CYP1B1 432Val/Val, COMT 158Val/Met,
(h) CYP1B1 432Val/Val, COMT 158Met/Met,
   has an increased risk of developing an estrogen-related cancer.

The present invention also provides a method for identifying a subject having an increased risk of developing an estrogen related cancer comprising determining which alleles of the genes encoding CYP1B1 and COMT are present in the genome of the subject so as to determine an estrogen metabolizing enzyme genotype for the individual, and correlating the estrogen metabolizing genotype of the individual to an increased risk of developing an estrogen related cancer, wherein a subject having an estrogen metabolizing enzyme genotype comprising a genotype corresponding to one of
(a) CYP1B1 432Val/Leu, CYP1B1 453Asn/Ser, COMT 158Val/Met;
(b) CYP1B1 432Val/Leu, CYP1B1 453Ser/Ser, COMT 158Val/Met;
(c) CYP1B1 432Val/Leu, CYP1B1 453Asn/Ser, COMT 158Met/Met;
(d) CYP1B1 432Val/Leu, CYP1B1 453Ser/Ser, COMT 158Met/Met;
(e) CYP1B1 432Val/Leu, CYP1B1 453Asn/Ser;
(f) CYP1B1 432Val/Leu, CYP1B1 453Ser/Ser;
(g) CYP1B1 432Val/Val, CYP1B1 453Asn/Ser, COMT 158Val/Met;
(r) CYP1B1 432Val/Val, CYP1B1 453Ser/Ser, COMT 158Val/Met;
(s) CYP1B1 432Val/Val, CYP1B1 453Asn/Ser, COMT 158Met/Met;
(t) CYP1B1 432Val/Val, CYP1B1 453Ser/Ser, COMT 158Met/Met;
(u) CYP1B1 432Val/Val, CYP1B1 453Asn/Ser;
(v) CYP1B1 432Val/Val, CYP1B1 453Ser/Ser;
(w) CYP1B1 432Val/Leu, CYP1B1 453Asn/Ser, COMT 158Val/Met;
(x) CYP1B1 432Val/Leu, CYP1B1 453Ser/Ser, COMT 158Val/Met;
(y) CYP1B1 432Val/Leu, CYP1B1 453Asn/Ser, COMT 158Met/Met;
(z) CYP1B1 432Val/Leu, CYP1B1 453Ser/Ser, COMT 158Met/Met;
(aa) CYP1B1 432Val/Val, CYP1B1 453Asn/Ser, COMT 158Val/Met;
(bb) CYP1B1 432Val/Val, CYP1B1 453Ser/Ser, COMT 158Val/Met;
(cc) CYP1B1 432Val/Val, CYP1B1 453Asn/Ser, COMT 158Met/Met; and
(dd) CYP1B1 432Val/Val, CYP1B1 453Ser/Ser, COMT 158Met/Met;
   has an increased risk of developing an estrogen-related cancer.

The present invention provides a method for identifying a subject having an increased risk of developing breast cancer comprising determining the presence in the subject of an allele of the gene encoding CYP1B1 that is correlated with an increased risk of developing breast cancer, wherein the allele comprises a nucleotide sequence encoding a CYP1B1 protein having an increased activity, whereby the presence of the allele identifies the subject as having an increased risk of developing breast cancer.

Further provided by the present invention is a method for identifying a subject having an increased risk of developing breast cancer comprising determining the presence in the subject of an allele of the gene encoding CYP1A1 that is correlated with an increased risk of developing breast cancer, wherein the allele comprises a nucleotide sequence encoding a CYP1A1 protein having an increased activity, whereby the presence of the allele identifies the subject as having an increased risk of developing breast cancer.

The present invention also provides a method for identifying a subject having an increased risk of developing breast cancer comprising determining the presence in the subject of an allele of the gene encoding COMT that is correlated with an increased risk of developing breast cancer, wherein the allele comprises a nucleotide sequence encoding a COMT protein having a decreased activity, whereby the presence of the allele identifies the subject as having an increased risk of developing breast cancer.

Also provided by the present invention is a method for identifying a subject as having an increased risk of developing breast cancer, comprising determining the nucleic acid sequence of the subject's CYP1B1 gene, whereby a subject having a CYP1B1 gene sequence which is correlated with an increased risk of developing breast cancer is identified as having an increased risk of developing breast cancer.

The present invention further provides a method for identifying a subject as having an increased risk of developing breast cancer, comprising determining the nucleic acid sequence of the subject's CYP1A1 gene, whereby a subject having a CYP1A1 gene sequence which is correlated with an increased risk of developing breast cancer is identified as having an increased risk of developing breast cancer.

The present invention provides a method for identifying a subject as having an increased risk of developing breast cancer, comprising determining the nucleic acid sequence of the subject's COMT gene, whereby a subject having a COMT gene sequence which is correlated with an increased risk of developing breast cancer is identified as having an increased risk of developing breast cancer.

Also provided by the present invention is a method of identifying an allele of a gene, wherein the allele is correlated with an increased risk of developing breast cancer, comprising:
(a) determining the nucleic acid sequence of the gene from a subject; and
(b) correlating the presence of the nucleic acid sequence of step (a) with the presence of breast cancer in the subject, whereby the nucleic acid sequence of the gene identifies an allele correlated with an increased risk of developing breast cancer.

The present invention provides a diagnostic test kit for determining the presence in a subject of an allele of the gene encoding CYP 1 B 1 that is correlated with an increased risk of developing breast cancer, comprising a means for identifying the nucleic acid sequence of the subject's CYP1B1 gene in a biological sample derived from the subject.

The present invention also provides a diagnostic test kit for determining the presence in a subject of an allele of the gene encoding CYP1A1 m1 that is correlated with an increased risk of developing breast cancer, comprising a means for identifying the nucleic acid sequence of the subject's CYP1A1 m1 gene in a biological sample derived from the subject.

The present invention further provides a diagnostic test kit for determining the presence in a subject of an allele of the gene encoding CYP1A1 m2 that is correlated with an increased risk of developing breast cancer, comprising a means for identifying the nucleic acid sequence of the subject's CYP1A1 m2 gene in a biological sample derived from the subject.

The present invention provides a diagnostic test kit for determining the presence in a subject of an allele of the gene encoding CYP1A1 m4 that is correlated with an increased risk of developing breast cancer, comprising a means for identifying the nucleic acid sequence of the subject's CYP1A1 m4 gene in a biological sample derived from the subject.

Also provided by the present invention is a diagnostic test kit for determining the presence in a subject of an allele of the gene encoding COMT that is correlated with an increased risk of developing breast cancer, comprising a means for identifying the nucleic acid sequence of the subject's COMT gene in a biological sample derived from the subject.

The present invention also provides a diagnostic test kit for determining the presence in a subject of a combination of alleles of the genes encoding CYP1B1, CYP1A1 m1, CYP1A1 m2, CYP1A1 m4 and COMT that is correlated with an increased risk of developing breast cancer, comprising a means for identifying the nucleic acid sequence of the CYP1B1, CYP1A1 m1, CYP1A1 m2, CYP1A1 m4 and COMT genes in a biological sample derived from the subject.

### BRIEF DESCRIPTION OF THE DRAWINGS

FIG. 1 is a diagram showing the metabolism of Estradiol (E₂). Oxidation of E2 is catalyzed by CYP1A1 and CYP1B1 to 2-OH and 4-OH catechol estrogens, respectively. The catechol estrogens are either methylated to methoxyestradiol (2-MeO E₂, 4-MeO E₂) by catechol-O-methyltransferase (COMT) or further oxidized to semiquinones (E₂-2,3SQ, E₂-3,4SQ) and quinones (E₂-2,3Q, E₂-3,4Q). The latter are either inactivated by glutathione conjugation catalyzed by glutathione transferases (GST) or form quinone-DNA adducts such as 4-OH E₂-1(_,_)-N7guanine. Alternatively, quinone-semiquinone redox-cycling may lead to oxidative DNA damage in the form of 8-hydroxydeoxyguanosine (8-OH-dG). The 4-OH catechol estrogens induce more DNA damage than 2-OH catechol estrogens as indicated by the thicker arrow. E₁ is metabolized in identical fashion.

FIG. 2 is a photograph of a SDS-polyacrylamide gel exposed to silver stain showing purified wild type (wt) and variant 1-5 CYP1B1 proteins.

FIGS. 3A-3C are graphs showing the spectrophotometric analysis of purified, recombinant wild-type CYP1B1.

FIG. 3A shows the CO-reduced difference spectrum of purified, recombinant wild-type CYP1B1.

FIG. 3B shows the absolute near uv-visible spectra of purified, recombinant wild-type CYP1B1.

FIG. 3C shows the derivative spectrum of purified, recombinant wild-type CYP1B1. The variant CYP1B1 proteins yielded similar spectra.

FIG. 4 is a graph showing the E2 concentration-dependent catalytic activity of wild type CYP1B1. Data are represented as means±standard deviations of duplicate assays: 4-OH-E2 (A) hydroxylation Km 40±8_M, kcat 4.4±0.4 min−1; 2-OH-E2 (Δ) hydroxylation Km 34±4_M, kcat 1.9±0.1 min⁻¹; 16_—OH-E2 (•) hydroxylation Km 39±6_M, kcat 0.30±0.02 min⁻¹.

FIG. 5 shows a summary of the general steps involved in implementing the MRD method. In step one, a set of n genetic and/or discrete environmental factors is selected from the pool of all factors. In step two, the n factor and their possible multifactor classes or cells are represented in n-dimensional space. In step three, each multifactor cell in n-dimensional space is labeled as high-risk if the ratio of cases to controls exceeds some threshold (e.g. #cases/#controls≧1.0) and low-risk if the threshold is not exceeded. In step four, the prediction error of each model is estimated using 10-fold cross-validation. Bars represent the distribution of cases (left) and controls (right) with each multifactor combination.

FIG. 6 shows a summary of the four-locus genotype combinations associated with high risk and with low risk sporadic breast cancer along with the corresponding distribution of cases (left bars) and controls (right bars) for each multilocus genotype combination. Note that the patterns of high risk and low risk cells differ across each of the different multilocus dimensions. That is evidence of epistasis or gene-gene interaction.

FIG. 7 shows a total ion chromatogram illustrating the separation of an equimolar mixture of estrogens, their metabolites and the deuterated internal standard (d4E2). The vertical dotted lines indicate the position of three different ion collection groups: 19-24.2 min [m/z 229, 257, 285, 287, 314, 315, 342, 343, 372, 373, 416, 417 and 420]; 2.4-26.5 min [m/z 257, 315, 342, 372, 373, 388, 389, 430, 431, 432, 446 and 447]; 26.2-31 min [m/z 283, 309, 311, 315, 345, 373, 414, 430, 431, 446, 447, 504 and 505]. The inset shows the single ion chromatograms (m/z 446, 414, 430 and 504) for the area within the dashed line on the total ion chromatogram where the peaks overlap. All compounds except 2-MeO-3-MeOE1 are chromatographed as TMS derivatives. The chromatography conditions are given in the text.

FIG. 8A shows an analysis of COMT genotypes by PCR amplification and digestion with BspHI followed by agarose gel electrophoresis shows bands of 160 bp for the Val/Val genotype (lane 2), 160, 125 and 35 bp for the Val/Met genotype (lane 3), and 135 and 25 bp for the Met/Met genotype (lane 4). The small 35 bp fragment is not visualized on this low melting agarose gel. Lane 1 shows the molecular size marker.

FIG. 8B shows SDS-PAGE of purified wild-type and variant COMT subjected to silver stain shows wild-type (lane 2) and variant (lane 3) COMT. Lane 1 contains the molecular weight marker.

FIG. 8C shows a Western immunoblot using anti-COMT antibody H6, showing recombinant wild type COMT (lane 1), recombinant variant COMT (lane 2), wild type COMT in ZR-75 cytosol (lane 3), and variant COMT in MCF-7 cytosol (lane 4).

FIG. 9A shows determination of kinetic parameters of COMT-mediated metabolism of catechol estrogen 2-OHE2. Data are represented as means±SD of two replicate assays. The points were fitted using nonlinear regression with the computer program GraphPad PRISM (San Diego, Calif.). The data reflect the best fit (judged by P value) according to a comparison of Michaelis-Menten and sigmoidal equations. The equations used were: Michaelis-Menten, v=(VₘₐₓS)/(Kₘ+S); sigmoidal, v=(VₘₐₓSⁿ)/(Kⁿₘ+Sⁿ).

FIG. 9B shows determination of kinetic parameters of COMT-mediated metabolism of catechol estrogen 4-OHE2. Data are represented as means±SD of two replicate assays. The points were fitted using nonlinear regression with the computer program GraphPad PRISM (San Diego, Calif.). The data reflect the best fit (judged by P value) according to a comparison of Michaelis-Menten and sigmoidal equations. The equations used were: Michaelis-Menten, v=(VₘₐₓS)/(Kₘ+S); sigmoidal, v=(VₘₐₓSⁿ)/(Kⁿₘ+Sⁿ).

FIG. 9C shows determination of kinetic parameters of COMT-mediated metabolism of catechol estrogen 2-OHE1. Data are represented as means±SD of two replicate assays. The points were fitted using nonlinear regression with the computer program GraphPad PRISM (San Diego, Calif.). The data reflect the best fit (judged by P value) according to a comparison of Michaelis-Menten and sigmoidal equations. The equations used were: Michaelis-Menten, v=(VₘₐₓS)/(Kₘ+S); sigmoidal, v=(VₘₐₓSⁿ)/(Kⁿₘ+Sⁿ).

FIG. 9D shows determination of kinetic parameters of COMT-mediated metabolism of catechol estrogen 4-OHE1. Data are represented as means±SD of two replicate assays. The points were fitted using nonlinear regression with the computer program GraphPad PRISM (San Diego, Calif.). The data reflect the best fit (judged by P value) according to a comparison of Michaelis-Menten and sigmoidal equations. The equations used were: Michaelis-Menten, v=(VₘₐₓS)/(Kₘ+S); sigmoidal, v=(VₘₐₓSⁿ)/(Kⁿₘ+Sⁿ).

FIG. 10 shows competitive COMT methylation of equimolar concentration (5 μM) of 2-OHE2,4-OHE2,2-OHE1, AND 4-OHE1. Data are represented as means±SD (n=3).

FIG. 11A shows a comparison of thermal stability of wild-type (open bar) and variant (shaded bar) COMT activity of products formed by methylation of 2-OHE2 and 4-OHE2. Data are represented as means±SD (n=3).

FIG. 11B shows a comparison of thermal stability of wild-type (open bar) and variant (shaded bar) COMT activity of products formed by methylation of 2-OHE1 and 4-OHE1. Data are represented as means±SD (n=3).

FIG. 12 shows ICELISA dose-response curving using COMT-GST standards over a range of 2.5-2500 ng/ml (R²=0.99). Data represent means of duplicate readings. The concentration of COMT in samples was obtained by correcting for the molecular weight contribution of GST (26 kDa) in the COMT-GST fusion protein (51 kDa).

FIG. 13A shows a comparison of wild-type COMT activity in ZR-75 cells (open bar) and variant COMT activity in MCF-7 cells (shaded bar) of products formed by methylation of 2-OHE2 and 4-OHE2. Data represent means±SD (n=3).

FIG. 13B shows a comparison of wild-type COMT activity in ZR-75 cells (open bar) and variant COMT activity in MCF-7 cells (shaded bar) of products formed by methylation of 2-OHE 1 and 4-OHE 1. Data represent means±SD (n=3).

FIG. 14 shows oxidative metabolism of E2 in two hypothetical women A and B with different CYP1A1, CYP1B1, COMT, GSTP1, and GSTT1 genotypes. The two women-represent the theoretical extremes in enzyme activity. Subject A has wild type genotypes for all enzymes, whereas subject B has all variant genotypes. Specifically, the CYP1B1 119Ser and the CYP1A1 462Val variants are associated with approximately 3-fold greater hydroxylation rates than the wild type enzymes while the COMT158Met and GSTP1 105Val variants are reduced 3-fold in activity compared to the respective wild types. GSTM1 null and GSTT1 null variants result in complete lack of activity. The wild type genotype has 100% activity. The difference in enzymatic activities is indicated by degree of arrow shading. The same pathway applies to E1.

FIG. 15. The GSTM1 gene (black box) at 1p13.3 consists of 8 exons, which range in size from 36 to 112 bp, while the introns vary from 87 to 2,641 bp (see top of diagram). The GSTM1 gene is embedded in a region with extensive homologies and flanked by two almost identical 4.2-kb regions (gray boxes). The GSTM1 null allele arises by homologous recombination of the left and right 4.2-kb repeats, which results in a 16-kb deletion containing the entire GSTM1 gene (see lower part of diagram). The point of deletion cannot be precisely localized because of the high sequence identity between the repeats. PCR primers 1 and 2 in exons 4 and 5, respectively (see arrowheads), were used to identify the presence of the wild-type allele, yielding a product of 273 bp. Primers 3, 4, 5, 6, 7 and 8 were designed to anneal outside the homology region. Using primers 3 and 4, the expected PCR product of 30 kb for the wild-type allele could not be amplified. However, the null allele yielded a 14-kb product, which allowed positive identification of the null allele. The arrowheads are not drawn to scale. The vertical arrows indicate the SwaI digestion sites.

FIG. 16. GSTM1 genotyping. (A) PCR analysis of GSTM1 null allele in three women yielded a single 14-kb product, which upon digestion with SwaI resulted in ˜12.4 and 1.6 kb fragments. Lanes are designated as u=undigested and d=digested products; M=DNA molecular weight markers. (B) Long-range PCR analysis of GSTM1 null allele in seven women. The 14-kb PCR product (lanes 3-7) indicates the presence of the null allele, while the lack of a product reveals its absence (lanes 1 and 2). (C) Short-range PCR analysis of GSTM1 wild-type allele in the same seven women. The 273-bp PCR product (lanes 1-5) indicates the presence of the wild-type allele, while the lack of a product reveals its absence (lanes 6 and 7). (D) The combined analysis of null and wild-type alleles indicates +/+ (lanes 1 and 2), +/− (lanes 3-5), and −/− (lanes 6 and 7) GSTM1 genotypes. (E) Separate long-range PCR analysis of the ERa gene yields a 14-kb fragment in every DNA sample indicating DNA integrity.

### DESCRIPTION OF THE PREFERRED EMBODIMENTS

The present invention may be understood more readily by reference to the following detailed description of preferred embodiments of the invention and the Examples included therein and to the Figures and their previous and following description.

As used in the specification and the appended claims, the singular forms “a,” “an” and “the” include plural referents unless the context clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

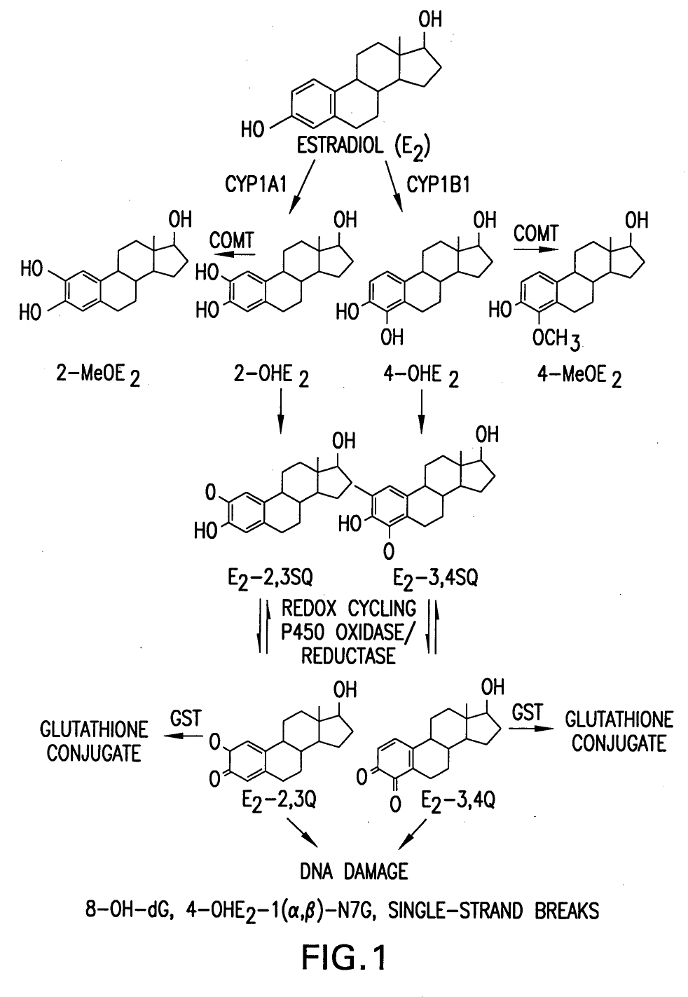

The present invention relates to the discovery that having a certain allele of an enzyme in the catechol estrogen pathway (see FIG. 1), (CYP1A1, CYP1 B1, COMT and GSTT1), can contribute to a subject's risk of developing an estrogen-related cancer, including, but not limited to, breast cancer and endometrial cancer, and that a subject's individual genotype for the genes encoding these five enzymes can be used to determine whether the subject has an increased or decreased risk of developing estrogen-related cancer.

Also, the present invention provides a method of analyzing the GSTM1 gene locus and provides a PCR assay to allow positive identification of the null allele. The GSTM1 null allele results from the deletion of the GSTM1 gene. In combination with the identification of the wild-type (wt) allele, true GSTM1 genotyping can be performed so that the associated inheritance patterns can be examined. Thus, the present invention provides a means to determine whether an individual has a GSTM1 genotype that is associated with breast cancer risk.

The present invention relates to the discovery that a subject with either a homozygous wild-type (+/+) or a heterozygous (+/−) GSTM1 genotype is a subject with an increased risk of developing cancer, for example, breast cancer, compared to a subject having a −/− gentotype. By “increased risk of developing cancer” is meant that an individual having one of the genotypes identified herein as being correlated with an increased risk of developing cancer, such as breast cancer, has an increased risk as compared to an individual who does not have one of the genotypes identified herein.

The individual used for comparison is preferably of a similar age and body mass; however, a comparison of an individual with another individual is not essential in order to determine if an individual has an increased risk of developing breast cancer or another cancer using the methods of the present invention. This is because the present application teaches that there is a statistical increase in risk for individuals with +/+ and +/− genotypes compared to individuals with a −/− genotype.

The present invention provides a method for identifying a subject having an increased risk of developing cancer, comprising determining the allele or alleles of the subject's GSTM1 gene, whereby a subject being homozygous for the wild-type allele or a subject being heterozygous for the wild-type and null alleles is identified as having an increased risk of developing cancer. As used herein, the wild-type version of GSTM1 (“wild-type GSTM1”) will be understood to refer to a GSTM1 enzyme having the amino acid sequence which is published in GenBank as having accession number J03817, and which is encoded by the nucleotide sequence published in GenBank as having accession number J03817, the contents of which are incorporated by reference herein. Furthermore, as will be understood by one of ordinary skill in the art, the reference herein to a null GSTM1 or a null allele means that no allele producing a functional GSTM1 enzyme is present in the individual.

Examples of cancers or cancerous tissues or organs that are contemplated by the present invention include, but are not limited to, breast, brain (e.g., astrocytoma, meningioma), colon, kidney, larynx, liver, lung, mouth, ovary, pituitary gland, pleura (e.g., mesothelioma), prostate, rectum, skin (e.g., basal cell carcinoma, melanoma), stomach, testis, urinary bladder, cervix and uterus. In addition, lymphoma, leukemia and other myeloproliferative disorders are contemplated. These cancers have been investigated in the context of GSTM1 genotype and are subject to risk-determination by the genotyping procedures described herein (11, 28, 30, 38, 57, 59, 73, 76, 87, 88, 89, 112, 119, 131, 151, 153, 159 and 161).

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and more preferably, a human.

The present invention also provides a method for identifying a subject having a decreased risk of developing cancer, for example, breast cancer, comprising determining the allele or alleles of the subject's GSTM1 gene, whereby a subject having an allele of the GSTM1 gene which is correlated with a decreased risk of developing cancer and which comprises a homozygous null allele is identified as having a decreased risk of developing cancer. Thus, a subject who is homozygous for the null allele of GSTM1 has a decreased risk of cancer compared to the risk of cancer a subject who is heterozygous for the wild-type allele and the null allele of the GSTM1 gene, or compared to a subject who is homozygous for the wild-type allele of the GSTM1 gene.

Given the teaching of the present invention, a person of skill using PCR would be able to identify a subject who is homozygous for the wild-type allele or a subject who is heterozygous for the wild-type allele and the null allele, or a subject who is homozygous for the null allele of GSTM1 by using the primers of the present invention as taught in the Examples below. In particular, a kit can be used to detect a subject who has an increased risk of breast cancer.

Another aspect of the present invention is a diagnostic kit for determining the presence in a subject of an allele of the gene encoding GSTM1 that is correlated with an increased risk of developing cancer, comprising means for detecting in a biological sample from the subject a homozygous wild-type genotype or a heterozygous wild-type/null genotype of GSTM1 and means for distinguishing a subject who is homozygous for the null allele of the GSTM1 gene. The kit contains a first nucleic acid primer pair having a nucleic acid with the sequence identified as SEQ ID NO:20 and a nucleic acid with the sequence identified as SEQ ID NO:21. The primer pair including SEQ ID NOs: 1 and 2 can amplify the wild-type allele and identify a subject who is homozygous for the wild-type allele or a subject who is heterozygous for the wild-type allele and the null allele of GSTM1. The kit also contains a second nucleic acid primer pair selected from the group of primer pairs consisting of a nucleic acid primer pair having a nucleic acid with the sequence identified as SEQ ID NO:22 and a nucleic acid with the sequence identified as SEQ ID NO:23; a nucleic acid primer pair having a nucleic acid with the sequence identified as SEQ ID NO:24 and a nucleic acid with the sequence identified as SEQ ID NO:25; and a nucleic acid primer pair having a nucleic acid with the sequence identified as SEQ ID NO:26 and a nucleic acid with the sequence identified as SEQ ID NO:27.

Another aspect of the present invention is a diagnostic kit for determining the presence in a subject of a homozygous null allele of the gene encoding GSTM1 that is correlated with a decreased risk of developing cancer, comprising means for identifying the allele of the subject's GSTM1 gene in a biological sample from the subject, wherein the identifying means comprises a nucleic acid primer pair selected from the group of primer pairs consisting of a nucleic acid primer pair having a nucleic acid with the sequence identified as SEQ ID NO:22 and a nucleic acid with the sequence identified as SEQ ID NO:23; a nucleic acid primer pair having a nucleic acid with the sequence identified as SEQ ID NO:24 and a nucleic acid with the sequence identified as SEQ ID NO:25; and a nucleic acid primer pair having a nucleic acid with the sequence identified as SEQ ID NO:26 and a nucleic acid with the sequence identified as SEQ ID NO:27. This kit can identify a subject with a reduced risk of cancer, for example breast cancer, compared to the risk of cancer in a subject who is homozygous for the wild-type allele of GSTM1, or a subject who is heterozygous for the wild-type allele and the null allele of GSTM1.

The primers of this invention specifically amplify a GSTM1 allele. By “specifically amplify” is meant that the primer amplifies only a nucleic acid from GSTM1. A primer of the invention has a nucleotide sequence that is specific for either cosmid clone cg/m1 from about nucleotide 15734 to about nucleotide 17595, or specific for cosmid clone cg/m12 from about nucleotide 8402 to about nucleotide 10260. A nucleotide sequence that is “specific” for cosmid clone cg/m1 from about nucleotide 15734 to about nucleotide 17595, or specific for cosmid clone cg/m12 from about nucleotide 8402 to about nucleotide 10260, is a nucleic acid that contains a sufficient number of contiguous nucleotides to be unique. To be unique, a primer of the invention must be of sufficient size to distinguish it from other known sequences, most readily determined by comparing any nucleic acid primer from about nucleotide 15734 to about nucleotide 17595 in cosmid clone cg/m1 or from nucleotide 8402 to about nucleotide 10260 in cosmid clone cgtm12 to the nucleotide sequences of nucleic acids in computer databases, such as GenBank. Such comparative searches are standard in the art. Typically, a unique (specific) nucleic acid useful as a primer or probe will be at least about 8 or 10 to about 30 or 35 nucleotides in length, depending upon the specific nucleotide content of the sequence. Additionally, primers can be, for example, at least about 40, 50, 75, 100, 200 or 500 nucleotides in length. Representative examples of primers obtained from the range of nucleotides 15734 to 17595 of cg/m1 (SEQ ID NO:28) include SEQ ID NOs:3, 5 and 7. Representative examples of primers obtained from the range of nucleotides 8402 to 10260 of cg/m12 (SEQ ID NO:29) include SEQ ID NOs:4, 6 and 8.

The present invention also provides primer pairs that can be used with various methods known in the art, for example PCR, to identify a subject as being homozygous for the wild-type allele (+/+) of GSTM1 or heterozygous for the wild-type allele and the null allele (+/−) of GSTM1 or homozygous for the null allele (−/−) of GSTM1. In one aspect of the invention, the primer pairs consist of an upstream primer and a downstream primer, each primer being from about 15 to about 35 nucleotides in length. Thus, each primer can be 14, 15, 16, 17, 18, 19, 20, 21, 22, 23, 24, 25, 26, 27 28, 29, 30, 31, 32, 33, 34, 35 or 36 nucleotides in length. Each upstream primer can be found specifically in cosmid clone cgtm 1 between about nucleotide 15734 and about nucleotide 17595. Each downstream primer can be found specifically in cosmid clone cgtm 12 between about nucleotide 8402 and about nucleotide 10260. A person of skill, using methods well-known in the art, can make an upstream primer of the invention from cosmid clone cg/m1 and a downstream primer of the invention from cg/m12. The nucleotide sequences of cosmid clone cgtm1 and cosmid clone cg/m12 are provided below. Examples of primer pairs of the invention include, but are not limited to, nucleic acids with the sequences identified as SEQ ID NO:20 and SEQ ID NO:21; SEQ ID NO:22 and SEQ ID NO:23; SEQ ID NO:24 and SEQ ID NO:25; and SEQ ID NO:26, and SEQ ID NO:27.

The present invention also relates to the discovery that the coordinated interaction of two or more enzymes in the catechol estrogen pathway (see FIG. 1), CYP1A1, CYP1B1, COMT and GSTT1, can contribute to a subject's risk of developing an estrogen-related cancer, including, but not limited to, breast cancer and endometrial cancer, and that a subject's individual genotype for the genes encoding these five enzymes can be used to determine whether the subject has an increased or decreased risk of developing estrogen-related cancer. This is due to the fact that genetic polymorphisms for each of the five enzymes exist, and can lead to changes in enzyme activity, expression, or stability which affect the metabolism of estrogen. Consequently, the combination of genes that a subject has for these enzymes can lead to the production of varying quantities of carcinogenic substances that are derived from estrogen.

Accordingly, the present invention provides a method for identifying a subject having an increased risk of developing an estrogen-related cancer comprising determining which alleles of the genes encoding CYP1A1, CYP1B1, COMT and GSTT1 are present in the genome of the subject, so as to determine an estrogen metabolizing enzyme genotype for the individual, and correlating the estrogen metabolizing genotype of the individual to the risk of developing an estrogen related cancer.

In a preferred embodiment, the estrogen related cancer is breast cancer. It can be shown that a subject having an estrogen metabolizing enzyme genotype comprising at least one of the following alleles has an increased risk of developing an estrogen-related cancer, including, but not limited to, breast cancer and endometrial cancer: CYP1A1 462Val; CYP1A1 461Asn; CYP1B1 432Leu; CYP1B1 453Asn; COMT 158Val; CYP1B1 48Gly; and null GSTT1.

Thus, in one embodiment, the invention relates to a method of identifying a subject having an increased risk of developing an estrogen-related cancer, wherein a subject having an estrogen metabolizing enzyme genotype comprising a genotype corresponding to one of: CYP1A1 462Ile/Val; CYP1A1 461Thr/Asn; CYP1B1 432Val/Leu; CYP1B1 453Asn/Ser; COMT 158Val/Met; CYP1B1 48Arg/Gly; CYP1A1 462Val/Val, CYP1A1 461Asn/Asn; CYP1B1 432Val/Val; CYP1B1 453Ser/Ser; COMT 158Met/Met; CYP1B1 48Gly/Gly; and null GSTT1 has an increased risk of developing estrogen-related cancer. In a preferred embodiment, the estrogen-related cancer is breast cancer.

It can be shown that a subject having an estrogen metabolizing enzyme genotype comprising a genotype corresponding to one of:
(a) CYP1B1 432Val/Leu, CYP1B1 453Asn/Ser;
(b) CYP1B1 432Val/Leu, CYP1B1 453Ser/Ser;
(c) CYP1B1 432Val/Leu, CYP1B1 48Arg/Gly;
(d) CYP1B1 432Val/Leu, CYP1B1 48Gly/Gly;
(e) CYP1B1 432Val/Leu, CYP1B1 119Ala/Ser;
(f) CYP1B1 432Val/Leu, CYP1B1 119 Ser/Ser;
(g) CYP1B1 432Val/Leu, COMT 158Val/Met;
(h) CYP1B1 432Val/Leu, COMT 158Met/Met;
(i) CYP1B1 432Val/Leu;
(j) CYP1B1 432Val/Val, CYP1B1 453Asn/Ser;
(k) CYP1B1 432Val/Val, CYP1B1 453Ser/Ser;
(l) CYP1B1 432Val/Val, CYP1B1 48Arg/Gly;
(m) CYP1B1 432Val/Val, CYP1B1 48Gly/Gly;
(n) CYP1B1 432Val/Val, CYP1B1 119Ala/Ser;
(O) CYP1B1 432Val/Val, CYP1B1 119 Ser/Ser;
(p) CYP1B1 432Val/Val, COMT 158Val/Met;
   (q) CYP1B1 432Val/Val, COMT 158Met/Met;
(r) CYP1B1 432Val/Val;
(s) CYP1B1 432Val/Leu, CYP1B1 453Asn/Ser, COMT 158Val/Met;
(t) CYP1B1 432Val/Leu, CYP1B1 453Ser/Ser, COMT 158Val/Met;
(u) CYP1B1 432Val/Leu, CYP1B1 453Asn/Ser, COMT 158Met/Met;
(v) CYP1B1 432Val/Leu, CYP1B1 453Ser/Ser, COMT 158Met/Met;
(w) CYP1B1 432Val/Leu, CYP1B1 48Arg/Gly, COMT 158Val/Met;
(x) CYP1B1 432Val/Leu, CYP1B1 48Gly/Gly, COMT 158Met/Met;
(y) CYP1B1 432Val/Leu, CYP1B1 119Ala/Ser, COMT 158Val/Met;
(z) CYP1B1 432Val/Leu, CYP1B1 119 Ser/Ser, COMT 158Met/Met;
(aa) CYP1B1 432Val/Leu, CYP1B1 453Asn/Ser;
(bb) CYP1B1 432Val/Leu, CYP1B1 453Ser/Ser;
(cc) CYP1B1 432Val/Leu, CYP1B1 48Arg/Gly;
(dd) CYP1B1 432Val/Leu, CYP1B1 48Gly/Gly;
(ee) CYP1B1 432Val/Leu, CYP1B1 119Ala/Ser;
(ff) CYP1B1 432Val/Leu, CYP1B1 119 Ser/Ser;
(gg) CYP1B1 432Val/Val, CYP1B1 453Asn/Ser, COMT 158Val/Met;
(hh) CYP1B1 432Val/Val, CYP1B1 453Ser/Ser, COMT 158Val/Met;
(ii) CYP1B1 432Val/Val, CYP1B1 453Asn/Ser, COMT 158Met/Met;
(j) CYP1B1 432Val/Val, CYP1B1 453Ser/Ser, COMT 158Met/Met;
(kk) CYP1B1 432Val/Val, CYP1B1 48Arg/Gly, COMT 158Val/Met;
(ll) CYP1B1 432Val/Val, CYP1B1 48Gly/Gly, COMT 158Val/Met;
(mm) CYP1B1 432Val/Val, CYP1B1 119Ala/Ser, COMT 158Met/Met;
(nn) CYP1B1 432Val/Val, CYP1B1 119 Ser/Ser, COMT 158Met/Met;
(O) CYP1B1 432Val/Val, CYP1B1 453Asn/Ser;
(pp) CYP1B1 432Val/Val, CYP1B1 453Ser/Ser;
(qq) CYP1B1 432Val/Val, CYP1B1 48Gly/Arg;
(rr) CYP1B1 432Val/Val, CYP1B1 48Gly/Gly;
(ss) CYP1B1 432Val/Leu, CYP1B1 453Asn/Ser, COMT 158Val/Met;
(tt) CYP1B1 432Val/Leu, CYP1B1 453Ser/Ser, COMT 158Val/Met;
(uu) CYP1B1 432Val/Leu, CYP1B1 453Asn/Ser, COMT 158Met/Met;
(vv) CYP1B1 432Val/Leu, CYP1B1 453Ser/Ser, COMT 158Met/Met;
(ww) CYP1B1 432Val/Val, CYP1B1 453Asn/Ser, COMT 158Val/Met;
(xx) CYP1B1 432Val/Val, CYP1B1 453Ser/Ser, COMT 158Val/Met;
(yy) CYP1B1 432Val/Val, CYP1B1 453Asn/Ser, COMT 158Met/Met;
(zz) CYP1B1 432Val/Val, CYP1B1 453Ser/Ser, COMT 158Met/Met;
(aaa) CYP1B1 432Val/Leu, CYP1B1 453Asn/Ser, COMT 158Val/Met, CYP1B1 119Ala/Ser;
(bbb) CYP1B1 432Val/Leu, CYP1B1 453Ser/Ser, COMT 158Val/Met, CYP1B1 119Ala/Ser;
(ccc) CYP1B1 432Val/Leu, CYP1B1 453Asn/Ser, COMT 158Met/Met, CYP1B1 119Ala/Ser;
(ddd) CYP1B1 432Val/Leu, CYP1B1 453Ser/Ser, COMT 158Met/Met, CYP1B1 119Ala/Ser;
(eee) CYP1B1 432Val/Val, CYP1B1 453Asn/Ser, COMT 158Val/Met, CYP1B1 119Ala/Ser;
(fff) CYP1B1 432Val/Val, CYP1B1 453Ser/Ser, COMT 158Val/Met, CYP1B1 119Ala/Ser;
(ggg) CYP1B1 432Val/Val, CYP1B1 453Asn/Ser, COMT 158Met/Met;
(hhh) CYP1B1 432Val/Val, CYP1B1 453Ser/Ser, COMT 158Met/Met, CYP1B1 119Ala/Ser;
(iii) CYP1B1 432Val/Leu, CYP1B1 453Asn/Ser, COMT 158Val/Met, CYP1B1 119Ser/Ser;
(jj) CYP1B1 432Val/Leu, CYP1B1 453Ser/Ser, COMT 158Val/Met, CYP1B1 119Ser/Ser;
(kkk) CYP1B1 432Val/Leu, CYP1B1 453Asn/Ser, COMT 158Met/Met, CYP1B1 119Ser/Ser;
(lll) CYP1B1 432Val/Leu, CYP1B1 453Ser/Ser, COMT 158Met/Met, CYP1B1 119Ser/Ser;
(mmm) CYP1B1 432Val/Val, CYP1B1 453Asn/Ser, COMT 158Val/Met, CYP1B1 119Ser/Ser;
(nnn) CYP1B1 432Val/Val, CYP1B1 453Ser/Ser, COMT 158Val/Met, CYP1B1 119Ser/Ser;
(ooo) CYP1B1 432Val/Val, CYP1B1 453Asn/Ser, COMT 158Met/Met, CYP1B1 119Ser/Ser;
(ppp) CYP1B1 432Val/Val, CYP1B1 453Ser/Ser, COMT 158Met/Met, CYP1B1 119Ser/Ser;
(qqq) CYP1B1 432Val/Leu, CYP1B1 453Asn/Ser, COMT 158Val/Met, CYP1B1 48Arg/Gly;
(rrr) CYP1B1 432Val/Leu, CYP1B1 453Ser/Ser, COMT 158Val/Met, CYP1B1 48Arg/Gly;
(sss) CYP1B1 432Val/Leu, CYP1B1 453Asn/Ser, COMT 158Met/Met, CYP1B1 48Arg/Gly;
(ttt) CYP1B1 432Val/Leu, CYP1B1 453Ser/Ser, COMT 158Met/Met, CYP1B1 48Arg/Gly;
(uuu) CYP1B1 432Val/Val, CYP1B1 453Asn/Ser, COMT 158Val/Met, CYP1B1 48Arg/Gly;
(vvv) CYP1B1 432Val/Val, CYP1B1 453Ser/Ser, COMT 158Val/Met, CYP1B1 48Arg/Gly;
(www) CYP1B1 432Val/Val, CYP1B1 453Asn/Ser, COMT 158Met/Met, CYP1B1 48Arg/Gly;
(xxx) CYP1B1 432Val/Val, CYP1B1 453Ser/Ser, COMT 158Met/Met, CYP1B1 48Arg/Gly;
(yyy) CYP1B1 432Val/Leu, CYP1B1 453Asn/Ser, COMT 158Val/Met, CYP1B1 48Gly/Gly;
(zzz) CYP1B1 432Val/Leu, CYP1B1 453Ser/Ser, COMT 158Val/Met, CYP1B1 48Gly/Gly;
(aaaa) CYP1B1 432Val/Leu, CYP1B1 453Asn/Ser, COMT 158Met/Met, CYP1B1 48Gly/Gly;
(bbbb) CYP1B1 432Val/Leu, CYP1B1 453Ser/Ser, COMT 158Met/Met, CYP1B1 48Gly/Gly;
(cccc) CYP1B1 432Val/Val, CYP1B1 453Asn/Ser, COMT 158Val/Met, CYP1B1 48Gly/Gly;
(dddd) CYP1B1 432Val/Val, CYP1B1 453Ser/Ser, COMT 158Val/Met, CYP1B1 48Gly/Gly;
(eeee) CYP1B1 432Val/Val, CYP1B1 453Asn/Ser, COMT 158Met/Met, CYP1B1 48Gly/Gly; and
(ffff) CYP1B1 432Val/Val, CYP1B1 453Ser/Ser, COMT 158Met/Met, CYP1B1 48Gly/Gly;
   has an increased risk of developing estrogen-related cancer. In a preferred embodiment, the estrogen-related cancer is breast cancer.

As used herein, the wild-type version of CYP1A1 (“wild-type CYP1A1”) will be understood to refer to a CYP1A1 enzyme having the amino acid sequence which is published in GenBank as having accession number X04300, and which is encoded by the nucleotide sequence published in GenBank as having accession number X04300, the contents of which are incorporated by reference herein.

Similarly, as used herein, wild-type version of CYP1B1 (“wild-type CYP1B1”) will be understood to refer to a CYP1B1 enzyme having the amino acid sequence which is published in GenBank as having accession number U03688, and which is encoded by the nucleotide sequence published in GenBank as having accession number U03688, the contents of which are incorporated by reference herein.

Furthermore, as used herein, the wild-type version of COMT (“wild-type COMT”) will be understood to refer to a COMT enzyme having the amino acid sequence which is published in GenBank as having accession number Z26491, and which is encoded by the nucleotide sequence published in GenBank as having accession number Z26491, the contents of which are incorporated by reference herein.

Furthermore, as used herein, the wild-type version of GSTT1 (“wild-type GSTT1”) will be understood to refer to a GSTT1 enzyme having the amino acid sequence which is published in GenBank as having accession number X79389, and which is encoded by the nucleotide sequence published in GenBank as having accession number X79389, the contents of which are incorporated by reference herein.

Unless otherwise stated, the residue numbers used in the allelic and genotypic notations herein represent an amino acid position in the wild-type version of the particular enzyme. However, a notation designating the amino acid found at a given residue number for a certain allele does not imply that the amino acid so noted is in fact found at that residue number in the wild-type version. The amino acid actually found at that residue number in the wild-type version of the enzyme can be determined by referring to the wild-type sequence for the enzyme, which can be found by referring to the sequences given for the particular enzyme at the GenBank accession numbers given above. It should also be noted that simply because an allelic or genotypic notation specifically identifies a particular amino acid as being found at a certain residue number, that does not imply that the presence of that amino acid represents a mutation at that position. Thus, for example, the genotype CYP1B1 119Ala/Ser corresponds to an individual having has one allele of CYP1B1 encoding an Ala at amino acid 119 of CYP1B1 (which happens to be the amino acid actually found at position 119 of wild-type CYP1B1), and one allele of CYP1B1 encoding a Ser at amino acid position 119 of CYP1B1 (which is not the amino acid actually found at position 119 of wild-type CYP1B1).

As used herein, the designation for a single allele of a gene, such as, e.g., CYP1B1 432Leu, represents the amino acid which is found at a specific amino acid residue of the relevant enzyme. Thus, CYP1B1 432Leu means that amino acid residue 432 of CYP1B1 is Leu.

As used herein, the designations for each genotype as used herein identify the amino acid found at the designated residue of the specified enzyme which is encoded by the nucleotide sequence of the first and the second allele for the enzyme. An individual may have two identical alleles encoding two identical versions of an enzyme (for example, as is designated by CYP1B1 432Leu/Leu) or two different alleles encoding two different variants of an enzyme (for example, as is designated by CYP1B1 432Val/Leu). Thus, CYP1B1 432Val/Leu means that an individual has one allele of CYP1B1 encoding a Leu at amino acid 432 of CYP1B1, and one allele of CYP1B1 encoding a Val at amino acid position 432 of CYP1B 1.

Furthermore, as will be understood by one of ordinary skill in the art, the reference herein to a null GSTT1 means that no allele producing a functional GSTT1 enzyme is present in the individual.

Unless otherwise indicated, where a genotype notation herein specifically names less than all of the enzymes selected from the group consisting of CYP1A1, CYP1B1, COMT and GSTT1, this means that both alleles of the unnamed enzymes are wild-type alleles. Thus, for example, the genotype “CYP1B1 432Val/Leu, COMT 158Val/Met” corresponds to an individual having 2 wild-type alleles for CYP1A1, GSTM1, and GSTT1, one CYP1B1 allele having a Leu at position 432, one CYP1B1 allele having a Val at position 432, one COMT allele having a Val at position 158, and one CYP1B1 allele having a Met at position 158.

It should be noted that a genotype may indicate the amino acids to be found at more than one position in the enzyme encoded by the allele. Thus, for example, the genotype “CYP1B1 432Val/Leu, CYP1B1 119Ala/Ser” corresponds to an individual having 2 wild-type alleles for CYP1A1, COMT and GSTT1, one CYP1B1 allele having a Leu at position 432 and an Ala at position 119, and one CYP1B1 allele having a Val at position 432 and a Ser at position 119.

In another embodiment, the invention provides a method for identifying a subject having an increased risk of developing breast cancer comprising determining the presence in the subject of an allele of the gene encoding CYP1B1 that is correlated with an increased risk of developing breast cancer, wherein the allele comprises a nucleotide sequence encoding a CYP1B1 protein having an increased activity, whereby the presence of the allele identifies the subject as having an increased risk of developing breast cancer. In a preferred embodiment, the allele correlated with increased risk is selected from the group consisting of CYP1B1 432Leu and CYP1B1 453Ser.

In another embodiment, the invention provides a method for identifying a subject having an increased risk of developing breast cancer comprising determining the presence in the subject of an allele of the gene encoding CYP1A1 that is correlated with an increased risk of developing breast cancer, wherein the allele comprises a nucleotide sequence encoding a CYP1A1 protein having an increased activity, whereby the presence of the allele identifies the subject as having an increased risk of developing breast cancer. In a preferred embodiment, the allele correlated with increased risk is selected from the group consisting of CYP1A1 462Val and CYP1A1 461 Asn.

In another embodiment, the invention provides a method for identifying a subject having an increased risk of developing breast cancer comprising determining the presence in the subject of an allele of the gene encoding COMT that is correlated with an increased risk of developing breast cancer, wherein the allele comprises a nucleotide sequence encoding a COMT protein having a decreased activity, whereby the presence of the allele identifies the subject as having an increased risk of developing breast cancer. In a preferred embodiment, the COMT allele correlated with increased risk is COMT 158Val.

In another embodiment, the invention provides a method for identifying a subject having an increased risk of developing breast cancer comprising determining the presence in the subject of an allele of the gene encoding GSTP1 that is correlated with an increased risk of developing breast cancer, wherein the allele comprises a nucleotide sequence encoding a GSTP1 protein having a decreased activity, whereby the presence of the allele identifies the subject as having an increased risk of developing breast cancer. In a preferred embodiment, the allele correlated with increased risk is selected from the group consisting of GSTP1 105Val and GSTP1 113 Val.

The present invention provides a method for identifying a subject as having an increased risk of developing breast cancer, comprising determining the nucleic acid sequence of the subject's CYP1B1 gene, whereby a subject having a CYP1B1 gene sequence which is correlated with an increased risk of developing breast cancer is identified as having an increased risk of developing breast cancer.

The present invention provides a method for identifying a subject as having an increased risk of developing breast cancer, comprising determining the nucleic acid sequence of the subject's CYP1A1 gene, whereby a subject having a CYP1A1 gene sequence which is correlated with an increased risk of developing breast cancer is identified as having an increased risk of developing breast cancer.

The present invention provides a method for identifying a subject as having an increased risk of developing breast cancer, comprising determining the nucleic acid sequence of the subject's COMT gene, whereby a subject having a COMT gene sequence which is correlated with an increased risk of developing breast cancer is identified as having an increased risk of developing breast cancer.

The present invention also provides a method for identifying a subject as having an increased risk of developing breast cancer, comprising determining the nucleic acid sequence of the subject's GSTP1 gene, whereby a subject having a GSTP1 gene sequence which is correlated with an increased risk of developing breast cancer is identified as having an increased risk of developing breast cancer.

In yet another embodiment, the invention provides a method for identifying a subject as having an increased risk of developing breast cancer, comprising:
a) correlating the presence of a specific allelic variant of the CYP1B1 gene with an increased risk of developing breast cancer; and
b) determining the nucleic acid sequence of the subject's CYP1B 1 gene, whereby a subject having a CYP1B1 gene which is correlated with an increased risk of developing breast cancer is identified as having an increased risk of developing breast cancer.

The invention also provides a method for identifying a subject as having an increased risk of developing breast cancer, comprising:
a) correlating the presence of a specific allelic variant of the CYP1A1 gene with an increased risk of developing breast cancer; and
b) determining the nucleic acid sequence of the subject's CYP1A1 gene, whereby a subject having a CYP1A1 gene which is correlated with an increased risk of developing breast cancer is identified as having an increased risk of developing breast cancer.

The invention also provides a method for identifying a subject as having an increased risk of developing breast cancer, comprising:
a) correlating the presence of a specific allelic variant of the COMT gene with an increased risk of developing breast cancer; and
b) determining the nucleic acid sequence of the subject's COMT gene, whereby a subject having a COMT gene which is correlated with an increased risk of developing breast cancer is identified as having an increased risk of developing breast cancer.

The invention also provides a method of identifying an allele of a gene correlated with an increased risk of developing breast cancer, wherein the gene encodes a protein selected from the group consisting of CYP1A1, CYP1B1, COMT and GSTT1, comprising:
a) determining the nucleic acid sequence of the gene from a subject; and
b) correlating the presence of the nucleic acid sequence of step (a) with the presence of breast cancer in the subject, whereby the nucleic acid sequence of the gene identifies an allele correlated with an increased risk of developing breast cancer.

By “increased risk of developing an estrogen-related cancer” and “increased risk of developing an estrogen-related cancer” is meant that an individual having one of the genotypes identified herein as being correlated with an increased risk of developing the estrogen-related cancer, such as breast cancer, has an increased risk as compared to an individual who does not have one of the genotypes identified herein.

The individual used for comparison is preferably of a similar age and body mass, however, these parameters are not essential in order to determine if an individual has an increased risk of developing breast cancer or another estrogen-related cancer using the methods of the present invention.

As is set forth in the examples, the invention also relates to a method for identifying a subject having a decreased risk of developing an estrogen related cancer such as breast cancer. Alleles and combinations thereof which are associated with having a decreased risk will be easily identifiable by one of ordinary skill upon review of the accompanying examples.

The methods of identifying a subject having an increased risk of developing breast cancer disclosed herein can be used for a number of purposes, such as determining whether a woman would be a suitable candidate for using birth control pills, or for estrogen replacement therapy at menopause.

The invention also provides a diagnostic test kit for determining the presence in a subject of an allele of the gene encoding CYP1B1 that is correlated with an increased risk of developing breast cancer, comprising a means for identifying the nucleic acid sequence of the subject's CYP1B1 gene in a biological sample derived from the subject. In a preferred embodiment, the identification means comprises a nucleic acid probe having the sequence given in SEQ ID NO: 5, and a nucleic acid probe having the sequence given in SEQ ID NO: 6.

The invention provides a diagnostic test kit for determining the presence in a subject of an allele of the gene encoding CYP1A1 m1 that is correlated with an increased risk of developing breast cancer, comprising a means for identifying the nucleic acid sequence of the subject's CYP1A1 m1 gene in a biological sample derived from the subject. In a preferred embodiment, the identification means comprises a nucleic acid probe having the sequence given in SEQ ID NO: 1, and a nucleic acid probe having the sequence given in SEQ ID NO: 2.

The invention also provides a diagnostic test kit for determining the presence in a subject of an allele of the gene encoding CYP1A1 m2 that is correlated with an increased risk of developing breast cancer, comprising a means for identifying the nucleic acid sequence of the subject's CYP1A1 m2 gene in a biological sample derived from the subject. In a preferred embodiment, the identification means comprises a nucleic acid probe having the sequence given in SEQ ID NO: 3, and a nucleic acid probe having the sequence given in SEQ ID NO: 4.

The invention also provides a diagnostic test kit for determining the presence in a subject of an allele of the gene encoding CYP1A1 m4 that is correlated with an increased risk of developing breast cancer, comprising a means for identifying the nucleic acid sequence of the subject's CYP1A1 m4 gene in a biological sample derived from the subject. In a preferred embodiment, the identification means comprises a nucleic acid probe having the sequence given in SEQ ID NO: 3, and a nucleic acid probe having the sequence given in SEQ ID NO: 2.

The invention also provides a diagnostic test kit for determining the presence in a subject of an allele of the gene encoding COMT that is correlated with an increased risk of developing breast cancer, comprising a means for identifying the nucleic acid sequence of the subject's COMT gene in a biological sample derived from the subject. In a preferred embodiment, the identification means comprises a nucleic acid probe having the sequence given in SEQ ID NO: 11 and a nucleic acid probe having the sequence given in SEQ ID NO: 12.

The invention also provides a diagnostic test kit for determining the presence in a subject of a combination of alleles of the genes encoding CYP1B1, CYP1A1 m1, CYP1A1 m2, CYP1A1 m4 and COMT, that is correlated with an increased risk of developing breast cancer, comprising a means for identifying the nucleic acid sequence of the CYP1B 1, CYP1A1 m1, CYP1A1 m2, CYP1A1 m4, COMT genes in a biological sample derived from the subject. In a preferred embodiment, the identifying means comprises a nucleic acid probe having the sequence given in SEQ ID NO: 5, a nucleic acid probe having the sequence given in SEQ ID NO: 6, a nucleic acid probe having the sequence given in SEQ ID NO: 1, a nucleic acid probe having the sequence given in SEQ ID NO: 2, a nucleic acid probe having the sequence given in SEQ ID NO: 3, a nucleic acid probe having the sequence given in SEQ ID NO: 4, a nucleic acid probe having the sequence given in SEQ ID NO: 7, a nucleic acid probe having the sequence given in SEQ ID NO: 8, a nucleic acid probe having the sequence given in SEQ ID NO: 11, and a nucleic acid probe having the sequence given in SEQ ID NO: 12.

The following examples are put forth so as to provide those of ordinary skill in the art with a complete disclosure and description of how the compositions and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, all nucleotide sequences are 5′ to 3′.

The present invention is more particularly described in the following examples which are intended as illustrative only since numerous modifications and variations therein will be apparent to those skilled in the art.

### EXAMPLE 1

### Genotypic Profile of Mammary Estrogen Metabolism as a Risk Factor for Breast Cancer

Material and Methods

Subjects. The study is based on 207 Caucasian women with primary invasive breast cancer who were treated at Vanderbilt University Medical Center, Nashville, Tenn. between 1982 and 1996. All patients had tumors of sufficient size (31.0 cm) to allow analysis of steroid receptors and extraction of DNA in addition to routine histopathological studies. Breast cancer patients were frequency matched by age to control patients hospitalized at Vanderbilt University Medical Center for various acute and chronic illnesses including trauma, transplant surgery, diabetes, cardiovascular and renal diseases. Reasons for exclusion of controls were breast cancer or other forms of malignancy as well as family history of breast cancer. Peripheral blood leukocytes served as source of DNA for the control subjects. Information regarding age, height, weight, and menstrual status was obtained from patients' medical records. Women were considered postmenopausal if they had no menses for at least 12 months or had undergone bilateral oophorectomy or, for women who had a hysterectomy without bilateral oophorectomy, were at least 55 years of age. The body mass index (BMI; weight in kg/height in m2) was calculated as a measure of obesity in all women except three patients and seven control subjects whose height or weight were not recorded.

DNA Analysis. DNA was isolated from all samples using a DNA extraction kit (Stratagene, La Jolla, Calif.). The enzyme genotype analysis was carried out by PCR and restriction endonuclease digestion (Table 1).

The following primers were used for analysis of alleles encoding the enzymes CYP1A1 (m1, m2, and m4 alleles), CYP1B1 (m1 and m2 alleles), GSTM1, GSTT1, and COMT:

The CYP1A1 m1 PCR primers used were forward primer A3 (SEQ ID NO.:1) 5′-GGCTGAGCAATCTGACCCTA and reverse primer A4 (SEQ ID NO.:2) 5′-GGCCCCAACTACTCAGAGGCT.

The CYP1A1 m2 PCR primers used were forward primer A1 (SEQ ID NO.:3) 5′-GAAAGGCTGGGTCCACCCTCT and reverse primer A2 (SEQ ID NO.:4) 5′-CCAGGAAGAGAAAGACCTCCCAGCGGGCCA.

The CYP1A1 m4 PCR primers used were forward primer A1 (SEQ ID NO.:3) 5′-GAAAGGCTGGGTCCACCCTCT and reverse primer A4 (SEQ ID NO.:2) 5′-GGCCCCAACTACTCAGAGGCT.

The CYP1B1 m1 PCR primers used were forward primer B1 (SEQ ID NO.:5) 5′-GTGGTTTTTGTCAACCAGTGG and reverse primer B2 (SEQ ID NO.:6) 5′-GCCCACTGAAAAAATCATCACTCTGCTGGTCAGGTGC.

The CYP1B1 m2 PCR primers used were forward primer B1 (SEQ ID NO.:7) 5′-CCTTGGCCGCTAAACCCGCTG and reverse primer B2 (SEQ ID NO.:8) 5′-CTGGCGCGTGAAGAAGTTGC.

The GSTT1 PCR primers used were forward primer T1 (SEQ ID NO.:9) 5′-TTCCTTACTGGTCCTCACATCTC and reverse primer T2 (SEQ ID NO.: 10) 5′-TCACCGGATCATGGCCAGCA.

COMT PCR primers used were forward primer C1 (SEQ ID NO.: 11) 5′-GCC GCCATCACCCAGCGGATGGTGGATTTCGCTGTC and reverse primer C2 (SEQ ID NO.: 12) 5′GTTTTCAGTGAACGTGGTGTG.

The B2 primer (SEQ ID NO.:6) contains a mutated nucleotide (underlined) to introduce a Cac8I site in order to reveal the polymorphism in codon 453 of the CYP1B1 gene. The specific amplification conditions for CYP1A1, CYP1B1 and GSTT1 and the subsequent restriction endonuclease analysis for CYP1A1 and CYP1B1 PCR fragments were described previously (Bailey, 1998a; Bailey, 1998b).

A BspHI restriction site was introduced into the C1 primer (SEQ ID NO.: 11) (see underlined nucleotide) to reveal the methionine allele in codon 158 of the COMT gene. BspHI is a 6-base cutter with a single recognition site on the PCR product of the methionine allele and no site on the valine allele. In contrast, the 4-base cutter NlaIII used by Lachman et al. (1996) cleaves three sites on the methionine allele and two sites on the valine allele yielding relatively small restriction fragments of 67 and 71 bp, which are not easily distinguished from each other. PCR was carried out in a total volume of 100 μl volume containing 0.5 μg genomic DNA, 10 mM Tris-HCl, pH 8.3, 50 mM KCl, 1.5 mM MgCl2, 200 μM each of the four deoxyribonucleotides, Amplitaq DNA polymerase (2.5 units; Perkin Elmer, Foster City, Calif.) and each primer at 25 μM. Amplification conditions consisted of an initial denaturing step followed by 30 cycles of 95° C. for 30 s, 64° C. for 1 min, and 72° C. for 6 min. A sample of the 160-base pair PCR product was size fractionated by electrophoresis in a 1.5% agarose gel and visualized by ethidium bromide staining. A portion (10%1) of the PCR product was subjected to restriction digest with BspHI (New England Biolabs, Beverly, Mass.) at 37° C. for 1 h. The digestion products were electrophoresed in a 4% low melting agarose gel (Amresco, Solon, Ohio) and visualized by ethidium bromide staining. Digestion with BspHI yielded bands of 160 bp for the Val/Val genotype, 160, 125 and 35 bp for the Val/Met genotype, and 125 and 35 bp for the Met/Met genotype. Each PCR contained internal controls for the respective gene and random re-testing of approximately 5% of samples yielded 100% reproducibility.

Statistical Methods. Logistic regression analyses were used to assess the effect of genotypes on breast cancer risk (Breslow, 1980). The odds ratios (ORs) from these analyses were adjusted for age by the case-control study design and by including age as a covariate in the regression models. In the models used for Table 4, genotype and BMI were also included as covariates together with appropriate genotype-BMI interaction terms. The possible effects of two-way interactions of different genes on breast cancer risk were examined for many combinations of genotypes as part of the data analysis for this study. The interactions presented in Table 6 were chosen on the basis of the magnitudes of the relative risks, their level of statistical significance, and the biologic plausibility of these interactions. Confidence intervals for these risks were estimated using Wald statistics (Stuart, 1991); P values were derived with respect to two-sided alternative hypotheses and were not adjusted for multiple comparisons.

The sample size of this study is large enough to detect several meaningful differences in breast cancer risk. Post hoc calculations (Dupont, 1990; Dupont, 1998) indicate that this study has 80% power to detect a breast cancer OR of 2.5 associated with lean women with either CYP1B1 m2 Asn/Ser or Ser/Ser genotypes versus lean women with CYP1B1 m2 Asn/Asn genotype. There is 80% power to detect an OR of 9.2. The accuracy of the OR estimates presented in this paper is best indicated by their associated 95% confidence intervals.

The distribution of genotype frequencies for CYP1A1, CYP1B1, COMT and GSTT1 is shown in Table 2. The distribution was similar in case patients and control subjects and no individual genotype had a significant effect on breast cancer risk. Since breast cancer risk and endogenous estrogen concentration are influenced by menopausal status and BMI, these variables had to be accounted for. Accordingly, the risk of breast cancer associated with individual genotypes stratified by menopausal status and BMI at the time of diagnosis of the case patients was examined (Table 3). The analysis was limited to postmenopausal women because the number of premenopausal women in this study was too small for meaningful multivariate statistical analysis. The reference groups for the CYP1A1 and CYP1B1 polymorphisms consisted of women who were homozygous for each of the more common alleles. Specifically, the leucine allele for CYP1B1 m1 was more common than the valine allele listed in the published amino acid sequence (Sutter, 1994). The high activity Val/Val genotype was designated as reference group for COMT. The reference groups for GSTT1 consisted of women who had one or both of the respective GST alleles. The reference group for each enzyme was assigned an OR of 1.0. Table 4 summarizes the associations of genotypes with postmenopausal breast cancer risk stratified by BMI. Lean women with the COMT Val/Met or Met/Met genotypes had a nearly four-fold reduction in risk of developing breast cancer (OR=0.26; P=0.003). Val/Met heterozygotes and Met/Met homozygotes each had similar risks of 0.24 (P=0.002) and 0.31 (P=0.03), respectively. The same COMT genotypes in obese women were associated with a 1.8-fold increase in risk of developing breast cancer, but this association was not statistically significant. The null GSTT1 genotype in lean women was associated with a three-fold higher risk of breast cancer (OR=3.13; P=0.007).

To investigate whether genotypic profiles of the enzymes involved in catechol estrogen metabolism are linked to the development of breast cancer, the association of combined genotypes with breast cancer risk was examined. Table 5 summarizes the statistically significant associations of combined genotypes with postmenopausal breast cancer risk. The CYP1B1 m1 Leu/Val or Val/Val genotypes in combination with either the CYP1B1 m2 Asn/Ser or Ser/Ser genotypes or the COMT Val/Met or Met/Met genotypes was associated with a reduction in breast cancer risk for women with a BMI below the median and an increase in risk for obese women.

Especially noteworthy is the 6-fold increase in risk of breast cancer for obese women with the combined CYP1B1 m1 Leu/Val or Val/Val and COMT Val/Met or Met/Met genotype (OR=6.07; P=0.02). In lean women, the combined CYP1B1 m2 Asn/Ser or Ser/Ser and COMT Val/Met or Met/Met genotype was associated with a 5-fold lower risk of developing breast cancer (OR=0.16; P=0.004).

Discussion

The CYP1B1 m1 Leu/Val or Val/Val genotypes in combination with either the CYP1B1 m2 Asn/Ser or Ser/Ser genotypes or the COMT Val/Met or Met/Met genotypes or the null GSTM1 genotype showed an association with susceptibility to breast cancer. This is of interest because CYP1B1 exceeds CYP1A1 in its catalytic efficiency as E2 hydroxylase, primarily due to its low Km for E2, and differs from CYP1A1 in its principal site of catalysis (Spink, 1992; Hayes, 1996). CYP1B1 has its primary activity at the C-4 position of E2 with a five-fold lower activity at C-2, whereas CYP1A1 has activity at the C-2, C-6_, and C-15_positions.

It was also observed that the CYP1B1 m1 Leu/Val or Val/Val genotypes in combination with either the CYP1B1 m2 Asn/Ser or Ser/Ser genotypes or the COMT Val/Met or Met/Met genotypes were associated with a reduction in postmenopausal breast cancer risk for women with a BMI below the median and an increase in risk for obese women. The difference in risk between lean and obese women may be attributable to a difference in circulating estrogen levels, which are influenced by body mass, especially in postmenopausal women, due to the conversion of androgens to estrogens by adipose tissue.

Catechol estrogens are inactivated by O-methylation, which is catalyzed by the ubiquitous COMT. The catalytic activity of COMT is affected by the methionine substitution for valine in codon 158 (Lachman, 1996). Individuals homozygous for the ‘Met’ allele have three- to four-fold lower COMT activity than those homozygous for ‘Val’ (Syvanen, 1997). Compared to the COMT Val/Val genotype, it was found that the Val/Met or Met/Met genotypes were associated with a reduction in breast cancer risk in lean, postmenopausal women and an increase in obese, postmenopausal women (Table 4). At least in the postmenopausal age group, it appears that the COMT Val/Met or Met/Met genotypes relative to the Val/Val genotype are associated with a reduced risk in lean women. When the data from the low and high BMI groups of the four studies (excluding the middle BMI tertile of Thompson's study) were combined, an OR of 0.57 (95% CI=0.40-0.81) (Table 6) was obtained. Moreover, the same pattern appears with other genotypes as well. As shown in Table 6, several of the combined genotypes are also associated with reduced risk in lean, postmenopausal women. In fact, the reduced risk associated with COMT variants among lean women is further reduced when combined with the CYP1B1 m2 Asn/Ser or Ser/Ser genotypes (OR=0.16; 95% CI=0.05-0.56). On the other hand, the risk in obese women is enhanced when combined with the CYP1B1 m1 Leu/Val or Val/al genotypes (OR=6.07; 95% CI=1.3-29). As stated, several studies have demonstrated significantly higher circulating estrogen levels in obese, postmenopausal women than in their lean counterparts (MacDonald, 1978; Moore, 1987; Potischman, 1996).

The present study demonstrates that a deletion polymorphism of GSTT1 is associated with an increased risk of breast cancer in postmenopausal women that is statistically significant among those with a BMI below the median 25.5 kg/m2 (OR=3.13; 95% CI=1.30-7.54).

### EXAMPLE 2

### Cytochrome P450 1B1 (CYP1B1) Pharmacogenetics: Association of Polymorphisms with Functional Differences in Estrogen Hydroxylation Activity

Materials and Methods

Construction of a CYP1B1 Bacterial Expression Plasmid. In order to facilitate expression and purification of CYP1B1, the hydrophobic N-terminal 25 amino acids of wild-type CYP1B1 (the nucleotide sequence were replaced by six histidine residues). This was accomplished by designing primers to contain BamHI and KpnI sites, respectively, at their 5′ ends to allow amplification of wild type and polymorphic CYP1B1 cDNA. The primers used were:

| | |
|---|---|
| 5′-CGG GAT CCC TCC TGT CGG TGC TGG | (SEQ ID NO.:13) |
| CCA CTG TGC ATG TGG | |
| and | |
| 5′-GGG GTA CCT TAT TGG CAA GTT TCC | (SEQ ID NO.:14) |
| TTG GCT TG. | |

The amplification reaction was carried out with 1 μg cDNA in a 100 μl volume containing 10 mM Tris-HCl (pH 8.3), 50 mM KCl, 1.5 mM MgCl12, 5 μl DMSO, 200 μM each of the four deoxyribonucleotides, native Pfu DNA polymerase (2.5 units; Stratagene; La Jolla, Calif.) and each oligonucleotide at 150 ng/ml. Amplification conditions consisted of a denaturing step at 95° C., annealing at 62° C., and extension at 72° C. for a total of 24 cycles. Each amplified cDNA was purified using the QIAquick PCR purification kit (QIAGEN; Valencia, Calif.), digested with BamHI and KpnI, and purified by centrifugation through a Chromaspin-100 column (Clontech; Palo Alto, Calif.). Each 1.6 kb PCR fragment was then ligated into the similarly digested vector pQE-30 (QIAGEN) which encodes the N-terminal hexahistidine tag. Each ligated vector/insert was transformed into XL1-Blue cells for amplification. The amplified plasmid DNA was then transformed into DH5_F′Iq using the methods described by the manufacturer. Colonies harboring the correct sequence (as judged by restriction digest and DNA sequencing) were picked and used to express the respective CYP1B1 protein.

Expression and Purification of Recombinant CYP1B1. Recombinant wild type and variant CYP1B1 proteins were expressed in Escherichia coli. Strain DH5_F′Iq yielded the highest expression levels. Transformed DH5_F′Iq cells were grown for 12 h at 37° C. in 50 ml modified TB medium containing 100 μg ampicillin/ml, 25 μg kanamycin/ml, 1 mM thiamine, and 10 mM glucose. The cells were then grown at 33° C. in the same medium with added trace elements as described until the OD600 was between 0.6 and 0.9. Mild induction with 8 mM lactose yielded optimal enzyme production, provided 0.5 mM_-aminolevulinic acid was added and cells were grown at 23° C. for 40 h while shaking at 150 rpm. After 40 h, cells were harvested by centrifugation at 6,500 g for 10 min and the P450 content in the bacterial cell lysate was determined by Fe2+-CO versus Fe2+ difference spectra. Spheroplasts were prepared with the use of lysozyme and disrupted by sonication. The pellet obtained after centrifugation at 10,000 g for 20 min was discarded and the microsomal membranes in the supernatant used as a source for purification. The membranes were pelleted by overnight centrifugation at 110,000 g and the resultant supernatant discarded because it generally contained <3% of the P450 content. The red 110 K pellet was resuspended in 200 ml solubilization buffer (100 mM NaPO4, pH 8.0, 0.4 M NaCl, 40% glycerol (v/v), 10 mM_-mercaptoethanol, 10_M aprotinin, 0.5% sodium cholate (w/v), 1.0% Triton N-101 (w/v)) and the suspension was stirred overnight. Centrifugation at 110,000 g for 90 min yielded a clear pellet, which was discarded, and a supernatant which contained most of the P450. The supernatant was applied to a pre-equilibrated Ni-NTA column (1 ml resin per 50 nmol enzyme). The column was washed with at least 50 column volumes of wash buffer (100 mM NaPO4, pH 8.0, 0.4 M NaCl, 40% glycerol (v/v), 10 mM b-mercaptoethanol, 0.25% sodium cholate (w/v), 10 mM imidazole), followed by a second wash with the same buffer containing 40 mM imidazole to remove unbound proteins and Triton N-101. The His-tagged protein was eluted with two column volumes of buffer (100 mM NaPO4, pH 8.0, 0.4 M NaCl, 40% glycerol (v/v), 10 mM_-mercaptoethanol, 0.25% sodium cholate (w/v), 400 mM imidazole), and the eluate dialyzed against dialysis buffer (100 mM NaPO4, pH 7.4, 0.25 M NaCl, 1 mM EDTA, 20% glycerol (v/v), 0.1 mM dithiothreitol). The purity of the protein was assessed by SDS-polyacrylamide gel electrophoresis and silver staining and by Western immunoblots using both anti-(oligo)His and anti-CYP1B1 antibodies.

Site-Directed Mutagenesis. Part of the initial studies of the CYP1B1 gene, including DNA sequence analysis, was carried out with human breast cancer cell lines. In analyzing the CYP1B1 gene in cell lines, it was determined that BT-20 cells contain the CYP1B1 sequence designated as wild type. Accordingly, wild-type CYP1B1 cDNA from BT-20 cells served as source for site-directed mutagenesis and the corresponding pQE-30 wild-type CYP1B1 plasmid was used as template to generate variant CYP1B1 cDNA encoding the substitutions in codon 48, 119, 432, and 453 (Table 7). Complementary 25 base oligonucleotide primers were synthesized to contain the selected mutated nucleotides in the center and purified by polyacrylamide gel electrophoresis. The following primers were used to amplify and introduce a polymorphism into exon 2 of CYP1B1 at codon 48:

| | |
|---|---|
| 5′-CAA CGG AGG CGG CAG CTC GGG TCC | (SEQ ID NO.:15) |
| GCG CC | |
| and | |
| 5′-GGC GCG GAC CCG AGC TGC CGCCTC | (SEQ ID NO.:16) |
| CGT TG. | |

The following primers were used to amplify and introduce a polymorphism into exon 2 of CYP1B1 at codon 119:

| | |
|---|---|
| 5′-CGA CCG GCC GTC CTT CGC CTC | (SEQ ID NO.:17) |
| CTT CCG | |
| and | |
| 5′-CGG AAG CAG GCG AAG GAC GGC | (SEQ ID NO.:18) |
| CGG TCG. | |

We utilized the primers in the QuikChange Site-Directed Mutagenesis method as specified by the manufacturer (Stratagene). After 12 PCR cycles with TurboPfu DNA polymerase the reaction was digested with DpnI and transformed into XL1-Blue cells. Successful mutagenesis was verified by nucleotide sequence analysis. Transformation into DH5_F′Iq cells, expression, and purification of variant CYP1B1 were performed as described above.

Spectrophotometric Analyses. All spectra were recorded using an Aminco DW2a/Olis instrument (On-Line Instrument Systems, Bogart, Ga.). Wavelength maxima were determined using the peak finder or second derivative software. The high-spin content was estimated from the second derivative spectrum of the ferric enzyme as described. P450 and cytochrome P420 concentrations were determined as described.

Assay of CYP1B1 E2 Hydroxylation Activity. Purified CYP1B1 (200 pmol) was reconstituted with a 2-fold molar amount of recombinant rat NADPH-P450 reductase (400 pmol), purified as previously described, and 60_g of L-_-dilauroyl-sn-glycero-3-phosphocholine in the presence of sodium cholate (0.005%, w/v) in 0.4 ml of 100 mM potassium phosphate buffer, pH 7.4, containing varying concentrations of E2 (2, 3, 6, 9, 12, 15, 20, 40, 60, 80, and 100_M) and 1 mM ascorbate. An NADPH-generating system consisting of 5 mM glucose 6-phosphate and 0.5 U of glucose-6-phosphate dehydrogenase/ml was added and reactions initiated by adding NADP+ to a final concentration of 0.5 mM. Reactions proceeded for 10 min at 37° C. with gentle shaking and then were terminated by addition of 2 ml CH₂Cl₂.

Extraction and Gas Chromatography/Mass Spectrometry Analysis of E2 and Metabolites. A deuterated internal standard (100_{—}1 of 8 mg/liter E2-2, 4, 16, 16-d4 in methanol; CDN Isotopes, Pointe-Claire, Quebec) was added and all steroids extracted into CH₂Cl₂ by vortex mixing for 30 s. 1.5 ml of the CH₂Cl₂ fraction was evaporated to dryness under air and volatile TMS derivatives prepared by heating the residue with 100_{—}1 of 50% NO-bis(trimethylsilyl)trifluoroacetamide/1% trimethyl chlorosilane in acetonitrile at 56° C. for 30 min. The TMS derivatives of E2 and its metabolites were separated by gas chromatography (H-P 5890, Hewlett-Packard, Wilmington, Del.) on a 5% phenyl methyl silicone stationary phase fused silica capillary column (30 m×0.2 mm×0.5_m film, HP5; Hewlett-Packard). Helium carrier gas was used at a flow of 1 ml/min. The injector was operated at 250° C., with 2_{—}1 injected in the splitless mode, with a purge (60 ml/min helium) time of 0.6 min. The oven temperature was held at 180° C. for 0.5 min, then raised at 6° C./min to 250° C. where it was held for 17 min, then raised to 300° C. at 8° C./min to give a total run time of 35.42 min. This program permitted adequate separation of a wide range of estrogen metabolites. Retention times for the TMS derivatives were: E2 and E2-d4 20.6, 2-OH-E2 26.6, 4-OH-E2 28.7, and 16a-OH-E2 30.3 min, respectively. The E1 mass spectrometer (H-P 5970) was operated in the selected ion monitoring mode from 18 to 34 min. Ions monitored were TMS2-E2-d4 420, 288, 330; TMS2-E2 416, 285, 326; TMS3-2-OH-E2 504, 373; TMS3-4-OH-E2 504, 373, 325; TMS3-16_-OH-E2 345, 311, 504. The instrument was calibrated by simultaneous preparation of an 11-point calibration over the range 0-10.5 nmol/tube of each compound. Sensitivity was determined to be between 0.02 and 0.04 mmol/tube (400-800 fmol on column) for the various compounds. Preparation of the TMS derivatives improved chromatography and sensitivity significantly. Derivation was performed at 56° C. since use of a higher temperature resulted in the loss of some estrogen derivatives (particularly the 2-OH metabolite of estrone). Derivation was demonstrated to be complete at 20 min as evidenced by the absence of detectable amounts of underivatized estrogens in the highest calibrator when the detector was operated in full scan mode. Absolute extraction efficiency for E2,2-OH-E2 and 4-OH-E2 at 3.5 mmol/tube was 119, 96, and 107% assessed by comparison to injections of spiked solvent samples onto the gas chromatograph. Internal standard added prior to extraction compensated for deviation from 100% recovery.

Statistical Analysis. Kinetic parameters (Km and kcat) were determined by nonlinear regression analysis using the computer program GraphPad PRISM (San Diego, Calif.).

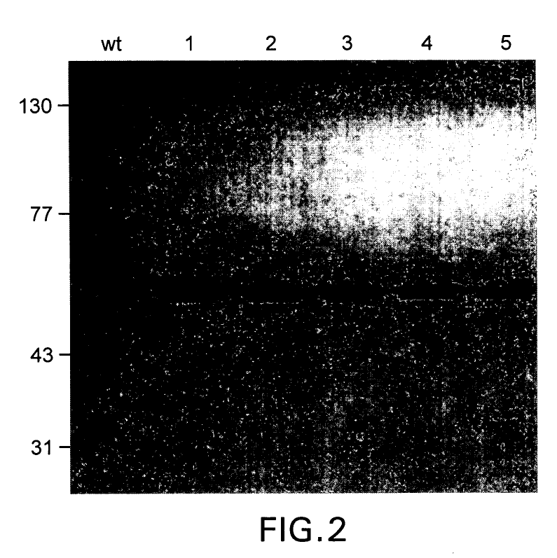

Initial attempts to express CYP1B1 in E. coli utilizing the pQE-30 vector yielded very low expression levels. Accordingly, the expression conditions to achieve higher levels of recombinant protein (400-800 nmol per liter) were modified. The modifications included the use of DH5aF′Iq instead of strains recommended by the manufacturer (Qiagen) and the induction of protein expression with lactose instead of isopropyl-b-D-thiogalactopyranoside. The protein modification strategy (i.e., replacement of the N-terminal hydrophobic segment) did not affect the intracellular localization of the recombinant protein in bacterial membranes. However, a much longer centrifugation period was required in the 110,000 g sedimentation step to pellet the majority of the expressed protein. The presence of the N-terminal hexahistidine allowed purification of the recombinant proteins with relatively high yields. Purified wild type and variant CYP1B1 were electrophoretically homogeneous as judged by SDS-polyacrylamide gel electrophoresis and silver staining, which revealed a single band at 55 kDa for all proteins (FIG. 2). Western immunoblots using both anti-(oligo)His and anti-CYP1B1 antibodies also yielded one major band at 55 kDa.

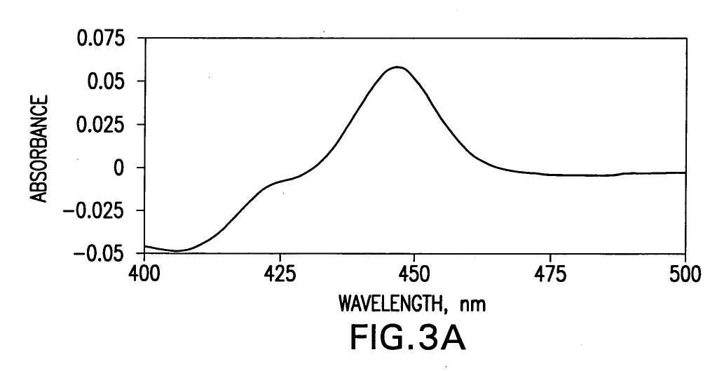

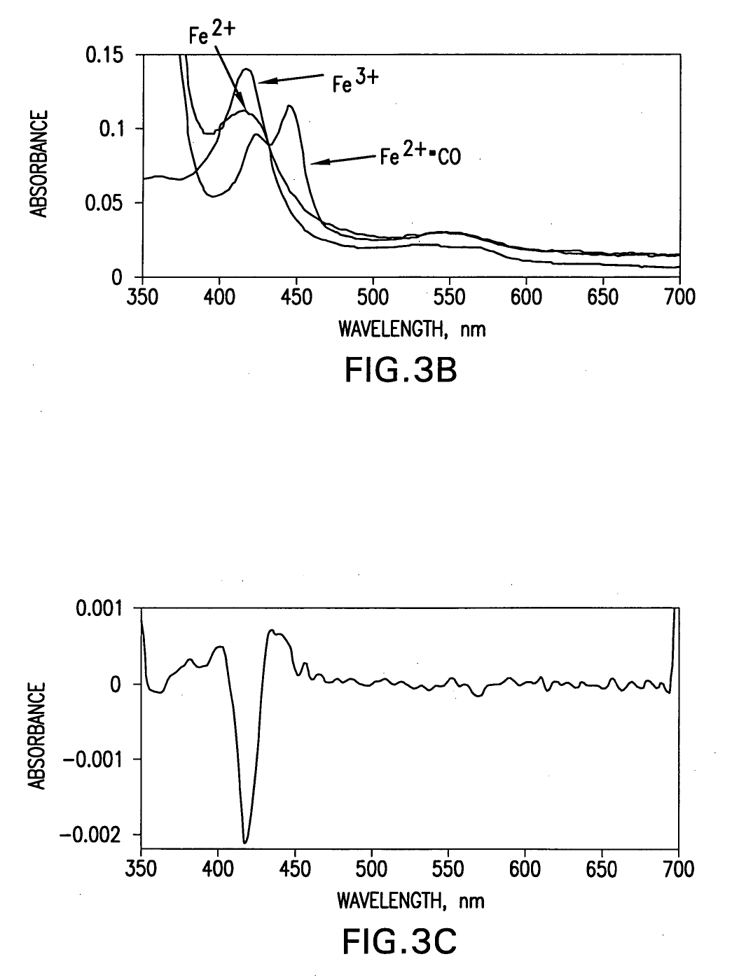

The reduced-CO difference spectrum of purified recombinant CYP1B1 had a _max at 450 nm and negligible amounts of cytochrome P420, the denatured form of the enzyme (FIG. 2). Examination of the absolute spectra of CYP1B1 revealed that the ferric protein was nearly all in the low-spin state. The low-spin character was further verified by examination of the second derivative spectrum (FIGS. 3A-3C).

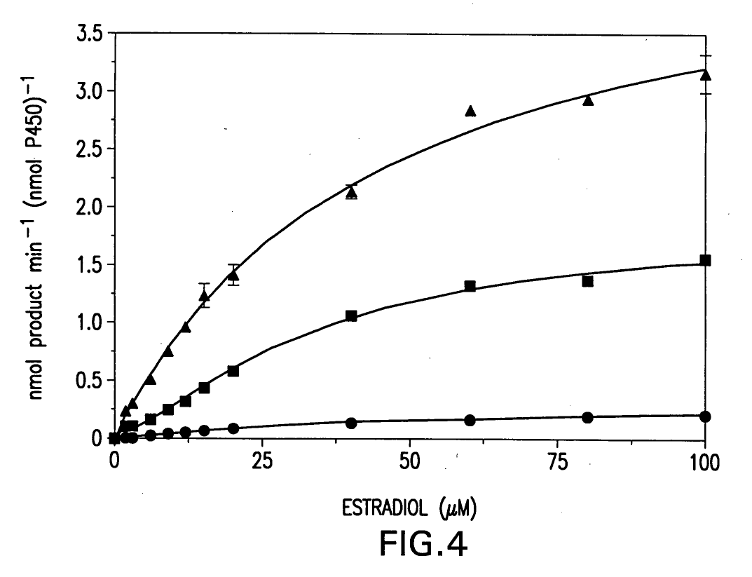

Wild type and variant CYP1B1 catalyzed E2 hydroxylation at C-2, C-4, and C-16_. Sodium cholate (0.005% w/v) was included in the reconstitution mixtures as suggested by Shimada et al. However, the exclusion of sodium cholate in separate experiments did not significantly affect the observed catalytic properties. The reaction kinetics were determined for each enzyme in duplicate at ten different concentrations of E2 (FIG. 4) and the resulting Km and kcat values are presented in Table 2. Wild type CYP1B1 formed 4-OH-E2 as main product (Km 40±8_M, kcat 4.4±0.4 min−1, kcat/Km 110 mM−1 min−1), followed by 2-OH-E2 (Km 34 ±4_M, kcat 1.9±0.1 min−1, k_{cat}/K_{m 55} mM−1 min−1) and 16_-OH-E2 (Km 39.4±5.7_M, kcat 0.30±0.02 min−1, k_{cat}/K_{m 7.6} mM−1 min−1). The CYP1B1 variants also formed 4-OH-E2 as main product, but displayed 2.4- to 3.4-fold higher catalytic efficiencies kcat/Km than the wild type enzyme, ranging from 270 mM−1 min−1 for variant 4 to 370 mM−1 min−1 for variant 2 (Table 8). The variant enzymes also exceeded wild type CYP1B1 with respect to 2- and 16_-hydroxylation activity, although the differences were smaller (Table 2). Overall, the 4-hydroxylation activity of the various enzymes was 2- to 4-fold higher than the 2-hydroxylation activity and 15- to 45-fold higher than the 16_-hydroxylation activity.

### EXAMPLE 3

### Multifactor Dimensionality Reduction Reveals High-Order Interactions among Estrogen Metabolism Genes in Sporadic Breast Cancer

Multifactor Dimensionality Reduction (MDR)

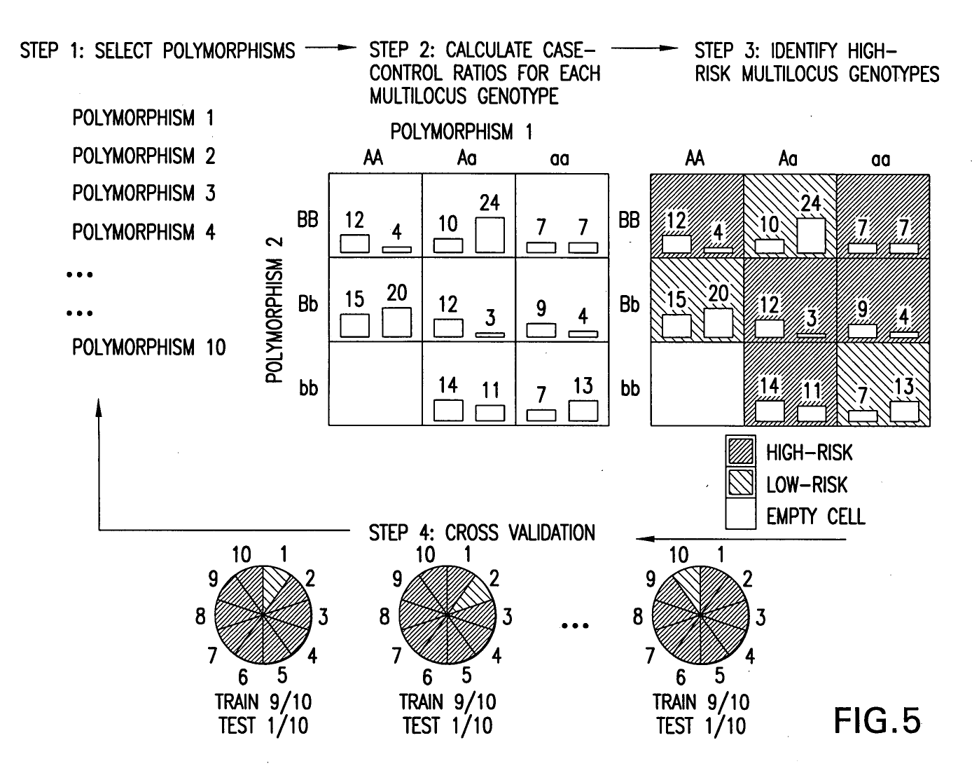

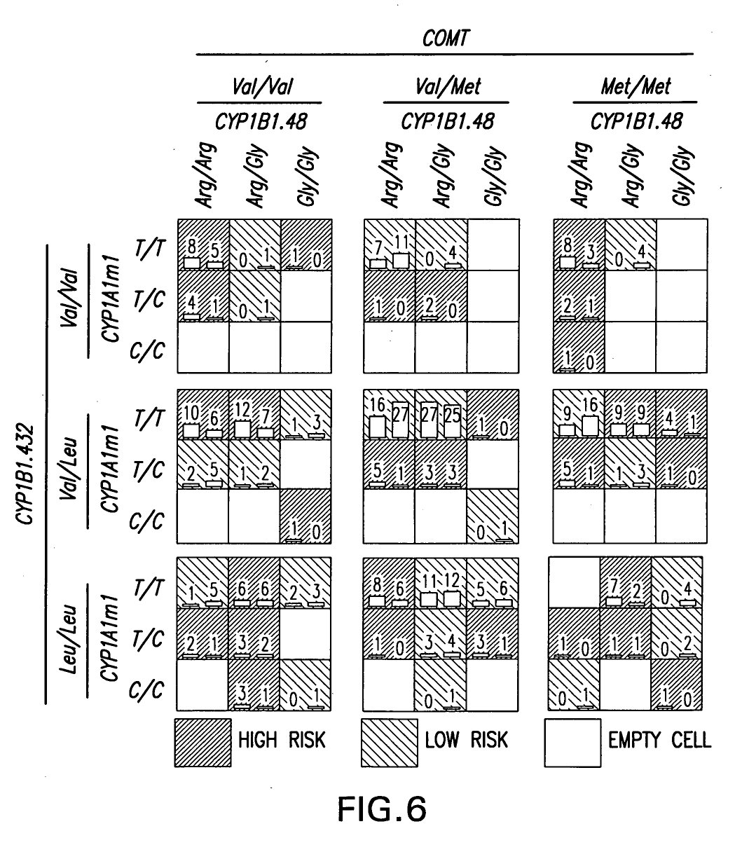

FIG. 5 illustrates the general steps involved in implementing the MDR method for case-control study designs. The same procedure is equally applicable to discordant sib-pair study designs. In step one, a set of n genetic and/or discrete environmental factors is selected from the pool of all factors. In step two, the n factors and their possible multifactor classes or cells are represented in n-dimensional space. For example, for two loci, each with three genotypes, there are nine two-locus genotype combinations. Then, the ratio of the number of cases (or affected sibs) to the number of controls (or unaffected sibs) is estimated within each multifactor class. In step three, each multifactor cell in n-dimensional space is labeled as high-risk if the ratio of cases to controls exceeds some threshold (e.g. #cases/#controls >1.0) and low-risk if the threshold is not exceeded. In this way, a model for cases and controls (or affected and unaffected sibs) is formed by pooling those cells labeled high-risk into one group and those cells labeled low-risk into another group. This reduces the n-dimensional model to one dimension (i.e. one variable with two multifactor classes; high risk and low risk). In this initial implementation of MDR, balanced case-control study designs are required. In step four, the prediction error of each model is estimated using 10-fold cross-validation. Here, the data are randomly divided into 10 equal parts. The MDR model is developed using each 9/10 of the data and then used to make predictions about the disease status of each 1/10 of the subjects left out. The proportion of subjects for which an incorrect prediction was made is an estimate of the prediction error. The 10-fold cross-validation is repeated 10 times and the prediction errors averaged to reduce the possibility of poor estimates of the prediction error due to chance divisions of the data set.

For more than two factors, steps one through four are repeated for each possible combination when computationally feasible. When the number of combinations to be evaluated exceeds computational feasibility, machine learning methods such as parallel genetic algorithms (Cantu-Paz 2000) must be employed. Among all of the two-factor combinations, a single model that maximizes the ratio of cases to controls for the high-risk group is selected. This two-locus model will have the minimum classification error among all of the two-locus models. Single best models are also selected from among each of the three-factor, four-factor, up to n-factor combinations. Among this set of best multifactor models, the combination of loci and/or discrete environmental factors that minimizes the prediction error is selected. Thus, the classification and prediction errors estimated using 10-fold cross-validation are used to select the final multifactor model. Hypothesis testing for this final model can then be carried out by evaluating the consistency of the model across cross-validation data sets. That is, how many times is the same MDR model identified in each 9/10 of the data? The reasoning is that a true signal (i.e. association) should be present in the data regardless of how it is divided. Statistical significance was determined by comparing the average cross-validation consistency from the observed data to the distribution of average consistencies under the null hypothesis of no associations derived empirically from 1,000 permutations. The null hypothesis was rejected when the upper-tail Monte Carlo p-value derived from the permutation test was less than or equal to 0.05.

Data Simulation

To evaluate the MDR method, four sets of 50 replicates of 200 cases and 200 controls using four different multilocus epistasis models were simulated. This number of replicates was selected to be large enough to provide validation of the method and small enough to allow exhaustive computational searches over all possible multilocus models. Unrelated subjects and genotypes for 10 unlinked diallelic loci were simulated using the Genometric Analysis Simulation Package or GASP (Wilson, 1996). Allele frequencies for each of the 10 loci were selected to match those in the breast cancer case-control sample. Hardy-Weinberg and linkage equilibrium were assumed. For the first model, we simulated a two-locus interaction effect using penetrance functions P(D|AAbb)=0.2, P(D|AaBb)=0.2, P(D|aaBB)=0.2, and P(D|others)=0 where D is disease and A, a, B, and b represent the alleles for the disease susceptibility loci. This is a well characterized model for epistasis in which risk of disease is dependent on whether exactly two deleterious alleles and two normal alleles are present from either or both loci (Frankel and Schork 1996; Li and Reich 2000). As described by Frankel and Schork (1996) and Li and Reich (2000), the independent main effects for the loci in this model are small. This two-locus epistasis model was extended to three-locus, four-locus, and five-locus epistasis models by adding corresponding homozygous or heterozygous genotypes to the penetrance functions described above. For example, for the three-locus epistasis model, penetrance functions P(D|AAbbcc)=0.2, P(D|AaBbcc)=0.2, P(D|aaBBcc)=0.2, P(D|aaBbCc)=0.2, P(D|AabbCc)=0.2, P(D|aabbCC)=0.2 were used. Thus, of the 10 total simulated loci, there were two, three, four, or five functional epistatic loci and up to eight nonfunctional loci.

Sporadic Breast Cancer Data

This study is based on 200 Caucasian women with sporadic primary invasive breast cancer who were treated at Vanderbilt University Medical Center, Nashville, Tenn. between 1982 and 1996. Informed consent for this study was obtained from all study subjects in accordance with the requirements of the Institutional Review Board of Vanderbilt University Medical School. Breast cancers were classified as sporadic or familial as per patient questionnaire. Patients with a family history of breast cancer have one or more first-degree relatives or two or more second-degree relatives with breast cancer. Patients not fulfilling these criteria were considered to have sporadic breast cancer. Sporadic breast cancer patients were frequency matched by age to control patients hospitalized at Vanderbilt University Medical Center for various acute and chronic illnesses. Reasons for exclusion of controls were breast cancer or other forms of malignancy as well as family history of breast cancer.

DNA was isolated from all samples using a DNA extraction kit (Gentra, Minneapolis, Minn.). The analysis was focused on CYP1A1 (chromosome 15q22-qter), CYP1B1 (2p21-22), COMT (22q 11.2) and GSTT1 (22q 11.2) because their enzyme products interact in the metabolism of estrogens to catechol estrogens and estrogen quinones. The COMT and GSTT1 genes are approximately 4 Mb apart on chromosome 22q11.2. Table 9 summarizes the polymorphisms in these genes that were analyzed by PCR and restriction endonuclease digestion. Genotype frequencies have been previously reported by our group (Bailey, 1998a, 1998b; Parl 2000) and others (Lavigne, 1997; Millikan, 1998; Thompson, 1998). The specific primers and amplification conditions and the subsequent restriction endonuclease analysis for CYP1A1, CYP1B1 and GSTT1 were described previously (Bailey, 1998a; Bailey, 1998b). COMT was amplified with primers C1: (SEQ ID.: 11) 5′-GCC GCC ATC ACC CAG CGG ATG GTG GAT TTC GCT GTC and C2: (SEQ ID.12) 5′-GTT TTC AGT GAA CGT GGT GTG. Each PCR contained internal controls for the respective gene and random re-testing of approximately 5% of the samples yielded 100% reproducibility.

Data Analysis

Prior to application of MDR to the sporadic breast cancer data set, the method was evaluated using the simulated multilocus data sets. For each of the 50 replicates generated by each of the four multilocus epistasis models, the MDR algorithm was applied as described above using a threshold of #cases/#controls >1.0. This threshold was selected such that multilocus genotype combinations would be considered high-risk if the number of cases with that particular combination was equal to or exceeded the number of controls. An exhaustive search of all possible two-locus, three-locus, up to nine-locus models was carried out. The 10-locus model was not evaluated since there is only one such model and the cross-validation consistency is always 10. Upon validation of the method, MDR was then applied to the sporadic breast cancer data set using the same threshold of #cases/#controls >1.0. Again, an exhaustive search of all possible two-locus, three-locus, up to nine-locus models was carried out.

Application of MDR to Simulated Data

Table 10 summarizes the mean of the cross-validation consistency and the prediction error obtained from the MDR analysis of each set of 50 simulated data sets for each gene-gene interaction model and each number of loci evaluated. The standard error of the mean is also reported. For each group of 50 simulated data sets, the mean prediction error was minimum and the mean cross-validation consistency was maximum for the particular multilocus model containing the correct two, three, four, or five genes. Additionally, the standard error of the mean prediction error and cross-validation consistency was minimum at the correct multilocus model. For example, in the case where a three-locus epistasis model was used to simulate the data sets the mean prediction error was minimum for the three-locus models at 12% with a standard error of 0.22%. The two-locus models had a mean prediction error of 21.91% (+/−0.33%) while the four-locus model had a prediction error of 12.37% (+/−0.24%). The mean prediction error for the four-locus model was much closer to that of the three-locus model because these models contained the correct three functional loci plus a false-positive locus while the two-locus models were missing one of the functional loci. Selecting the smaller three-locus model with the lower prediction error is consistent with statistical parsimony (i.e. smaller models are better because they are easier to interpret). For the three-locus models in this example, the cross-validation consistency was always 10. That is, the same three-locus model was found in each possible 9/10 of the data. These results suggest that, for this particular epistasis model, the cross-validation strategy is a reasonable approach to identifying the correct multilocus model. Further, the threshold of #cases/#controls >1.0 was reasonable for this epistasis model.

The Monte Carlo p-values for each of the correctly identified models were all less than 0.001. The estimated power to identify the correct multilocus model was 78% for the two-locus model, 82% for the three-locus model, 94% for the four-locus model, and 90% for the five-locus model. It is interesting that the power tends to increase as higher-order interactions are modeled. This may be a real phenomenon or it might be due to the fact that fewer non-functional loci out of the 10 total that were simulated were present. These results suggest that, for this particular epistasis model, the MDR approach has reasonable power to identify high-order gene-gene interactions with a sample size of 200 cases and 200 controls.

Application of MDR to the Breast Cancer Data

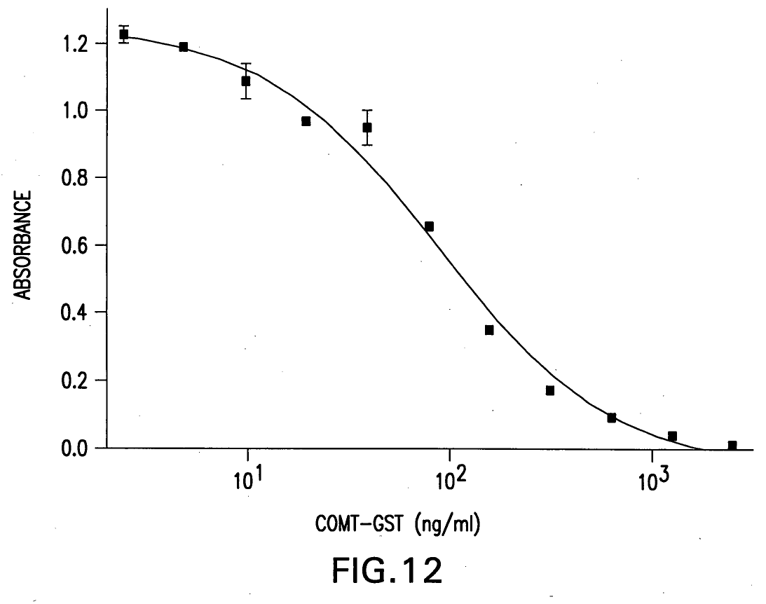

Table 11 summarizes the cross-validation consistency and prediction error obtained from MDR analysis of the sporadic breast cancer case-control data set for each number of loci evaluated. One four-locus model had a minimum prediction error of 46.73 and a maximum cross-validation consistency of 9.8 that was significant at the 0.001 level as determined empirically by permutation testing. Thus, under the null hypothesis of no association, it is highly unlikely to observe a cross-validation consistency as great or greater than 9.8 for this four-locus model. The four-locus model included the COMT, CYP1B1 codon 432, CYP1B1 codon 48, and CYP1A1m1 polymorphisms. FIG. 12 summarizes the four-locus genotype combinations associated with high risk and with low risk along with the corresponding distribution of cases and controls for each multilocus genotype combination. Note that the patterns of high risk and low risk cells differ across each of the different multilocus dimensions. This is evidence of epistasis or gene-gene interaction. That is, the influence of each genotype at a particular locus on risk of disease is dependent on the genotypes at each of the other three loci. Previous analysis of this data set using logistic regression revealed no statistically significant evidence of independent main effects of any of the 10 polymorphisms (Bailey, 1998a, 1998b).

### EXAMPLE 4

### Catechol-O-Methyltransferase (COMT)-Mediated Metabolism of Catechol Estrogens: Comparison of Wild-Type and Variant COMT Isoforms Chemicals

Catechol estrogens (2-OHE2,2-OHE1,4-OHE2,4-OHE1) and methoxyestrogens (2-MeOE2,2-MeOE1,4-MeOE2,4-MeOE1,2-OH-3-MeOE2,2-OH-3-MeOE1, 2-MeO-3-MeOE2, 2-MeO-3-MeOE1) were obtained from Steraloids, Newport, R1. Deuterated E2 (E2-2, 4, 16, 16-d4) was obtained from CDN Isotopes, Pointe-Claire, Quebec.

Cell Lines. Breast cancer cell lines ZR-75 and MCF-7 were obtained from the American Type Culture Collection, Rockville, Md. and grown under recommended culture conditions. DNA was isolated using a DNA extraction kit (Gentra, Minneapolis, Minn.).

DNA Polymorphism Analysis. COMT was amplified with primers C1: (SEQ ID NO.: 11) 5′-GCCGCCATCACCC AGCGGATGGTGGATTTCGCTGTC and C2: (SEQ ID NO.: 12) 5′GTTTTCAGTGAACGTGGTGTG. PCR was carried out in a total volume of 100 μl containing 0.5 μg genomic DNA, 10 mM Tris-HCl, pH 8.3, 50 mM KCl, 1.5 mM MgCl₂, 200 1M each of the four deoxyribonucleotides, Amplitaq DNA polymerase (2.5 units; Roche Diagnostics, Indianapolis, Ind.) and each primer at 25 μM. Amplification conditions consisted of an initial denaturing step followed by 30 cycles of 95° C. for 30 s, 64° C. for 1 min, and 72° C. for 6 min. A sample of the 160-base pair PCR product was size fractionated by electrophoresis in a 1.5% agarose gel and visualized by ethidium bromide staining. A portion (10 μl) of the PCR product was subjected to restriction digest with BspHI (New England Biolabs, Beverly, Mass.) at 37° C. for 1 h. The digestion products were electrophoresed in a 4% low melting agarose gel (Amresco, Solon, Ohio) and visualized by ethidium bromide staining.

Expression and Purification of Recombinant S-COMT. Breast cancer cell lines ZR-75 (Val/Val) and MCF-7 (Met/Met) served as a source for wild type and variant S-COMT cDNA, respectively. Primers were designed to contain SacI and SalI sites, respectively, at their 5′ ends to allow amplification of wild type and variant S-COMT cDNA and ligation of the PCR product into vector pQE-30 (QIAGEN; Valencia, Calif.), which encodes an N-terminal hexahistidine tag for subsequent purification (27). Each ligated vector/insert was transformed into XL1-Blue cells for amplification. The amplified plasmid DNA was then transformed into Escherichia coli strain DH5αF′Iq and colonies harboring the correct sequence (verified by restriction digest and complete DNA sequencing) were selected to express the respective S-COMT protein. Transformed DH5αF′Iq cells were grown in modified TB medium containing ampicillin (100 μg/ml), and kanamycin (25 μg/ml). When the OD₆₀₀ was between 0.4 and 0.6, cells were induced with 12 mM lactose and grown at 30° C. for 16 h while shaking at 200 rpm. Cells were harvested by centrifugation at 5,000 g for 20 min and spheroplasts prepared by exposure to lysozyme. The spheroplasts were disrupted by sonication in 100 mM Tris-HCl, pH 8.0, 0.3 M NaCl, 1 mM EDTA, 20% glycerol (v/v), 10 mM α-mercaptoethanol, 5 mM MgCl₂, and 10 μM each of aprotinin, leupeptin, and pepstatin. The pellet obtained after centrifigation at 10,000 g for 20 min was discarded and the supernatant centrifuged overnight at 110,000 g. The resultant supernatant was applied to a pre-equilibrated Ni-NTA column (1 ml resin per 50 mmol enzyme). The column was washed with at least 50 column volumes of wash buffer (100 mM NaPO₄, pH 8.0, 0.4 M NaCl, 20% glycerol (v/v), 10 mM β-mercaptoethanol, 5 mM MgCl₂, 20 mM imidazole). The His-tagged protein was eluted with two column volumes of buffer (100 mM NaPO₄, pH 7.4, 0.25 M NaCl, 20% glycerol (v/v), 10 mM β-mercaptoethanol, 5 mM MgCl₂, 100 mM imidazole), and the eluate dialyzed against dialysis buffer (100 mM NaPO₄, pH 7.4, 0.25 M NaCl, 0.1 mM EDTA, 20% glycerol (v/v), 0.1 mM dithiothreitol, 2 mM MgCl₂). The purity of the protein was assessed by SDS-polyacrylamide gel electrophoresis and silver staining and by Western immunoblot using anti-COMT antibodies.

Selection of COMT-Specific Single Chain Fragment Variable (ScFv) Antibodies from a Phage-Displayed Recombinant Antibody Library. A rodent phage-displayed recombinant antibody library (˜2.9×10⁹ members), generated by the Vanderbilt University Molecular Recognition Unit core facility, was used to obtain ScFv recombinant antibodies specific for COMT. All ScFv stemming from the recombinant antibody library had been cloned into E. coli TG1 cells using the pCANTAB5E phagemid vector (Amersham Pharmacia Biotech Inc., Piscataway, N.J.). Expressed ScFv display a tag recognized by the Pharmacia Anti-E tag and HRP/Anti-E tag monoclonal antibodies. The Anti-E tag antibody can be used to detect ScFv bound to antigens in assays and can also be used to affinity-purify ScFv from bacterial extracts. Initial selections with purified His-COMT did not yield ScFv antibodies with sufficient affinity for use in immunoassays. Therefore, another tag, glutathione S-transferase (GST), was attached using the plasmid pGEX-4T (Amersham Pharmacia Biotech Inc.) to produce the recombinant purified fusion protein COMT-GST. Three rounds of phage antibody selection were performed using one ml of COMT-GST immobilized on Nunc Maxisorb tubes at 100 μg COMT-GST/ml PBS for the first, 10 μg/ml for the second, and 1 μg/ml for the third round of selection. Tubes and phage antibodies were blocked in 0.09-0.1% Tween 20 in PBS prior to selections. Phage antibodies were eluted from COMT-GST-coated tubes with 1 ml of 100 mM triethanolamine for the first two rounds of selection and with His-COMT at 10 μg/ml PBS for the third round. Eluted phage antibodies were used to infect E. coli TG1 cells, which served as bacterial source for phage-displayed or soluble recombinant antibody production.

Immune Complex Enzyme-Linked Immunosorbant Assay (ICELISA) to Determine ScFv Antigen-Specificity. The ICELISA protocol, which accompanies Amersham Pharmacia's HRP/Anti-E tag conjugate, was used to detect and determine antigen-specificity of ScFv produced by bacterial colonies. All assays were carried out in 384 well microtiter plates with individual wells either left uncoated or coated with 50 μl of COMT-GST, His-COMT or GST at 5 μg/ml PBS.

Preparation and Purification of ScFv from Bacterial Periplasmic Extracts. Bacteria were grown overnight at 30° C. in 250 ml of 2xYT medium with 100 μg/ml ampicillin and 2% glucose shaking at 100 rpm. Bacteria were centrifuged to pellet cells, resuspended in 2xYT medium with 100 μg/ml ampicillin and 1 mM isopropyl-β-D-thiogalacto-pyranoside, incubated and centrifuged as before. To prepare periplasmic extracts, bacterial pellets were resuspended sequentially in 10 ml of TES (0.2 M Tris-HCl, pH 8.0, 0.5 mM EDTA, 0.5 M sucrose), 15 ml of one-fifth TES (0.04 M Tris-HCl, pH 8.0, 0.1 mM EDTA, 0.1 M sucrose) and placed on ice for 1 h or at −70° C. until needed. Recombinant ScFv were purified from periplasmic extracts by affinity chromatography using an Amersham Pharmacia RPAS Purification Module according to the manufacturer's instructions.

Western Immunoblot of COMT. Purified recombinant His-COMT and COMT in breast cancer cell cytosol were resolved by SDS polyacrylamide gel electrophoresis and transferred to nitrocellulose. Nitrocellulose filters were blocked for 1 h with 3% nonfat dry milk in PBS (3% NFDM). The HRP/Anti-E tag conjugate was diluted 1:4,000 in 3% NFDM, mixed with an equal volume ScFv in periplasmic extract, applied to COMT samples on nitrocellulose blots, and incubated for 1 h at room temperature. Blots were washed for 30 min in PBS containing 0.05% Tween 20 after which ScFv bound to COMT were visualized on film using an HRP-enhanced chemiluminescent substrate.

Competitive ICELISA to Quantify COMT. Based on preliminary assays, six bacterial clones produced ScFv that interacted with COMT-GST and His-COMT, but not with GST. The ScFv bacterial clone designated C3 was selected based on optimal absorbance readings at 405 nm: 2.646 (COMT-GST), 2.702 (His-COMT), 0.136 (GST) and 0.208 (blank well). The competitive ICELISA was carried out at room temperature in a 384-well microtiter plate coated for 2 h with purified COMT-GST at 0.5 μg/ml PBS, 50 μl/well. Wells were emptied, filled with PBS containing 0.1% Tween 20 (PBST) and blocked for 15 min. Known concentrations of COMT-GST were mixed with C3-HRP/Anti-E immune complex (composed of purified C3, diluted to 2.7 μg/ml, and HRP/Anti-E conjugate, diluted 1:8,000 in 3% NFDM) to obtain a standard curve. Cytosol samples containing COMT were diluted 1/10 in C3-HRP/Anti-E immune complex. Following a 90-min incubation, samples and COMT-GST standards were added in duplicate to the COMT-GST-coated microtiter wells, at 50 μl/well. After a 1 h incubation, wells were washed seven times with PBS containing 0.05% Tween 20. Wells were tapped dry and 50 μl of 2,2′azino-bis(3-ethylbenzthiazoline-6-sulfonic acid) (ABTS) and hydrogen peroxide added for color development and absorbance readings at 405 nm using a BIO-TEK ELx800NB plate reader (BIO-TEK Instruments Inc., Winooski, Vt.). The plate reader's KCjr software was used to generate a standard curve, based on a four-parameter fit, and calculate COMT concentrations in samples.

Assay of COMT Activity. Purified recombinant His-COMT (300 pmol) was reconstituted in 0.5 ml of 100 mM KPO4, pH 7.4, containing 5 mM MgCl₂, 10 mM (β-mercaptoethanol, and 200 μM SAM. Reactions were initiated by adding varying concentrations of each individual catechol estrogen (2, 3, 6, 9, 12, 15, 20, 40, 60, 80, and 100 μM). Blanks contained all compounds except SAM. Reactions proceeded for 10 min at 37° C. with gentle shaking and then were terminated by addition of 2 ml CH₂Cl₂. To determine COMT activity in breast cancer cells, ZR-75 and MCF-7 cells were harvested at confluency and homogenized in 100 mM KP04, pH 7.4, 5 mM MgCl₂, 10 mM β-mercaptoethanol. Following ultracentrifugation of the cell homogenate (110,000 g, 30 min, 4° C.), the supernatant cytosol was divided into aliquots for ICELISA, protein determination (BCA assay; Pierce, Rockford, Ill.), and COMT assay. The latter was carried out in the presence of 200 μM SAM and 100 μM catechol estrogen for 20 min at 37° C. and then terminated by addition of CH₂Cl₂. The concentration of endogenous catechol and methoxy estrogens was below the limit of detection by gas chromatography/mass spectrometry.

Thermal Inactivation. COMT thermal stability was measured as described by Scanlon. Specifically, aliquots of recombinant wild type and variant COMT were heated at 48° C. for 15 min while control samples were kept on ice. The heated samples were returned to ice before measurement of enzyme activity. Thermal stabilities were expressed as heated/control (H/C) ratios, a commonly used measure of enzyme thermal stability.

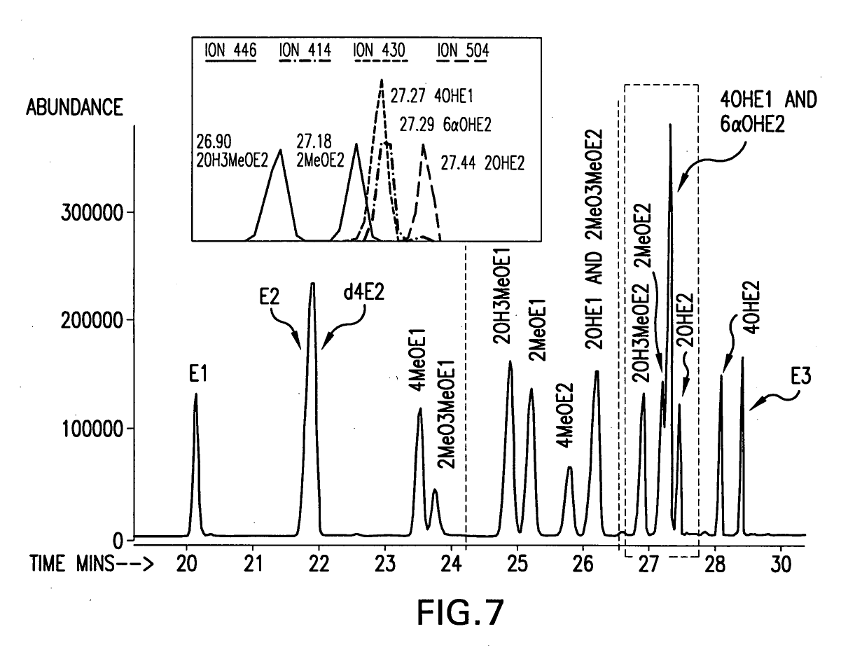

Extraction and Gas Chromatography/Mass Spectrometry Analysis of Catechol Estrogens. A deuterated internal standard (100 μl of 8 mg/liter E2-d4 in methanol) was added and all estrogens extracted into the CH₂Cl₂ by vortex mixing for 30 s. 1.5 ml of the CH₂Cl₂ fraction was evaporated to dryness under air and volatile TMS derivatives prepared by heating the residue with 100 μl of 50% NO-bis(trimethylsilyl)trifluoroacetamide/1% trimethyl chlorosilane in acetonitrile at 56° C. for 30 min. The TMS derivatives of the estrogen metabolites were separated by gas chromatography (H—P 5890, Hewlett-Packard, Wilmington, Del.) on a 5% phenyl methyl silicone stationary phase fused silica capillary column (30 m×0.2 mm×0.5 μm film, HP5; Hewlett-Packard). Helium carrier gas was used at a flow of 1 ml/min. The injector was operated at 250° C., with 2 μl injected in the splitless mode, with a purge (60 ml/min helium) time of 0.6 min. The oven temperature was held at 180° C. for 0.5 min, then raised at 6° C./min to 250° C. where it was held for 17 min, then raised to 300° C. at 8° C./min to give a total run time of 35.42 min. This program permitted adequate separation of a wide range of estrogen metabolites. Retention times (in min) for the TMS derivatives were E1 20.13, E2 and E2-d4 21.89, 4-MeOE1 23.52, 2-MeO-3-MeOE1 (underivatized) 23.75, 2-OH-3-MeOE1 24.87, 2-MeOE1 25.2, 4-MeOE2 25.78, 2-OHE1 and 2-MeO-3-MeOE2 26.19, 2-OH-3-MeOE2 26.9, 2-MeOE2 27.18, 4-OHE1 27.27, 6a-OHE2 27.29, 2-OHE2 27.44, 4-OHE2 28.06, E3 28.38. The E1 mass spectrometer (H—P 5970) was operated in the selected ion monitoring mode from 18 to 30 min. Ions monitored were TMS-E1 342, 257, 343; TMS₂-E2-d4 420, 421, 287; TMS₂-E2 416, 417, 285; TMS-4-MeOE1, TMS-2-OH-3-MeOE1, TMS-2-MeOE1 and TMS-3-MeO-4-OHE1 372, 373, 342; 2-MeO-3-MeOE1 314, 315, 229; TMS₂-4-MeOE2, TMS₂-2-OH-3-MeOE2, TMS₂-2-MeOE2 and TMS₂-3-MeO-4-OHE2 446, 447, 315; TMS₂-2-OHE1 430, 431, 432; TMS-2-MeO-3-MeOE2 388, 389, 257; TMS₂-4-OHE1 430, 431, 345; TMS₂-6α-OHE2 414, 283, 309; TMS₃-2-OHE2 and TMS₃-4-OHE2 504, 505, 373; TMS₃-E3 504, 505, 311 (FIG. 7). The instrument was calibrated by simultaneous preparation of an 11-point calibration over the range 0-22 nmol/tube of each compound. Sensitivity was determined to be between 0.02 and 0.04 nmol/tube (400-800 finol on column) for the various compounds. Preparation of the TMS derivatives improved chromatography and sensitivity significantly. Derivatization was performed at 56° C. since use of a higher temperature resulted in the loss of some estrogen derivatives (particularly the 2-OH metabolite of estrone). Derivatization was demonstrated to be complete at 20 min as evidenced by the absence of detectable amounts of underivatized estrogens in the highest calibrator when the detector was operated in full scan mode. Absolute extraction efficiency for E2,2-OH-E2 and 4-OH-E2 at 3.5 nmol/tube was 119, 96, and 107% assessed by comparison to injections of spiked solvent samples onto the gas chromatograph. Internal standard added prior to extraction compensated for deviation from 100% recovery for all investigated compounds.

Statistical Analysis. Kinetic parameters (Kₘ and k_{cat}) for the enzyme reactions were determined by nonlinear regression analysis using the computer program GraphPad Prism (San Diego, Calif.).

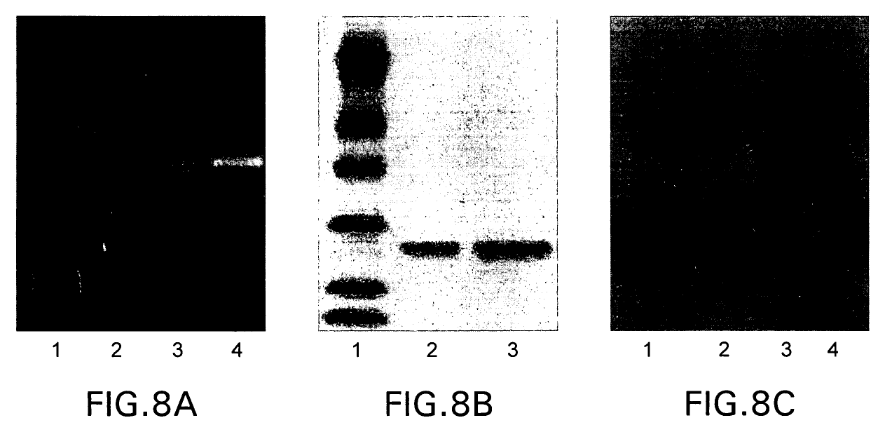

PCR and restriction endonuclease digestion were performed to identify the wild-type and variant COMT allelels. A BspHI restriction site was introduced into the C1 primer (see ‘Materials and Methods’, underlined nucleotide) to reveal the methionine allele in codon 108 of the COMT gene. BspHI is a 6-base cutter with a single recognition site on the PCR product of the methionine allele and no site on the valine allele. In contrast, the 4-base cutter NlaIII used by Lachman et al. cleaves three sites on the methionine allele and two sites on the valine allele yielding relatively small restriction fragments of 67 and 71 bp, which are not easily distinguished from each other. Digestion of the COMT PCR product with BspHI yielded bands of 160 bp for the Val/Val genotype, 160, 125 and 35 bp for the Val/Met genotype, and 125 and 35 bp for the Met/Met genotype (FIG. 8A). Breast cancer cell lines ZR-75 (Val/Val) and MCF-7 (Met/Met) served as source for wild type and variant S-COMT cDNA, respectively. His-tagged wild type and variant S-COMT were expressed and purified by Ni-NTA chromatography. Each recombinant protein was electrophoretically homogeneous as judged by SDS-PAGE and silver staining, which revealed a single band at Mᵣ 25,000 (FIG. 8B). COMT-specific ScFv antibodies were developed to further characterize the recombinant COMT and to demonstrate the presence of wild type and variant COMT in breast cancer cell lines ZR-75 and MCF-7, respectively. Initial attempts to select for phage-displayed COMT-specific ScFv using purified His-COMT yielded antibodies whose affinity was too low for use in immunoassays. Therefore, recombinant, purified COMT-GST was prepared to generate antibodies with greater affinity. The ScFv bacterial clone designated H6 proved optimal, yielding the following ICELISA absorbance readings: 2.551 (COMT-GST), 0.441 (His-COMT), 0.141 (GST), and 0.151 (blank well). The Western immunoblot using anti-COMT antibody H6 showed one major band at Mᵣ 25,000 for recombinant wild type and variant COMT (FIG. 8C, lanes 1 and 2). Similarly, wild type and variant COMT in cytosol of ZR-75 and MCF-7 cells, respectively, migrated predominantly as one band (FIG. 8C, lanes 3 and 4). However, the cytosol protein migrated slightly higher than the recombinant protein, probably due to post-translational modification.

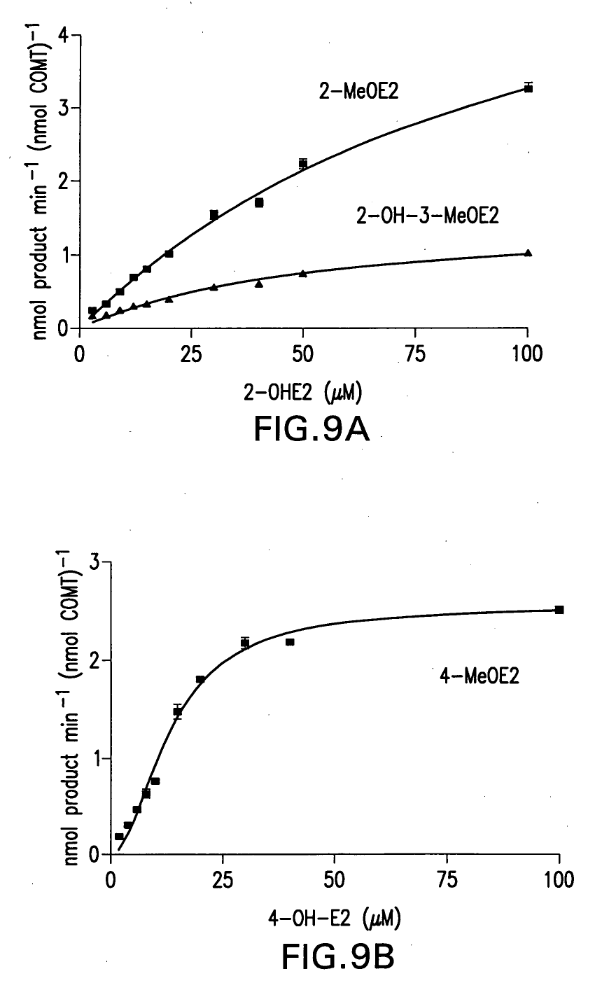

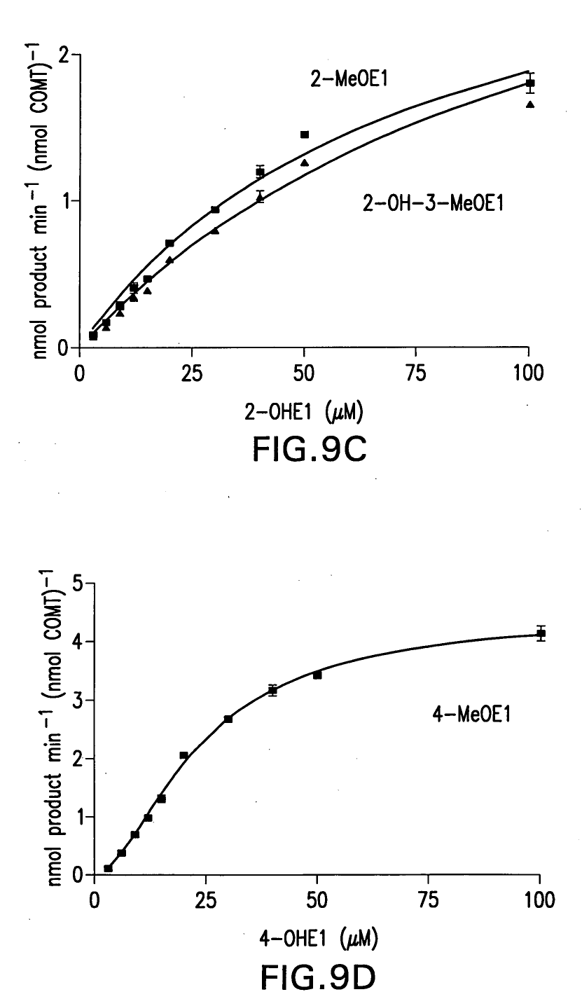

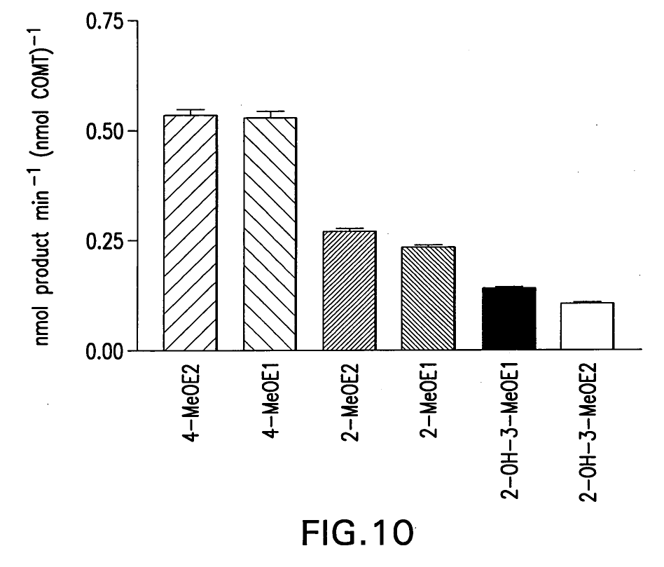

COMT activity was assessed by determining the methylation of the substrates 2-OHE2, 4-OHE2,2-OHE1, and 4-OHE1 (FIG. 9). The reaction kinetics were determined in two replicate experiments at ten different concentrations of each substrate. The resulting Kₘ and k_{cat} are presented in Table 12. COMT catalyzed the formation of monomethyl ethers at 2-OH, 3-OH, and 4-OH groups. Dimethyl ethers were not observed. In the case of 2-OHE2 and 2-OHE1, methylation occurred at 2-OH and 3-OH groups, resulting in the formation of 2-MeOE2 and 2-OH-3-MeOE2, and 2-MeOE1 and 2-OH-3-MeOE1, respectively. In contrast, in the case of 4-OHE2 and 4-OHE1, methylation occurred only at the 4-OH group, resulting in the formation of 4-MeOE2 and 4-MeOE1, respectively. 3-MeO-4-OHE2 and 3-MeO-4-OHE1 were not observed. As shown in FIG. 9, the rates of methylation of 2-OHE2 and 2-OHE1 yielded typical hyperbolic patterns, whereas 4-OHE2 and 4-OHE1 exhibited a sigmoid curve pattern. Overall, COMT displayed the highest catalytic efficiencies k_{cat}/Kₘ in the formation of 4-MeO products (142 and 126 mM⁻¹ min⁻¹), followed by the 2-MeO products (63 and 45 mM⁻¹ min⁻¹), and lastly the 3-MeO products (29 and 38 mM⁻¹ min⁻¹) (Table 12). Competition experiments using an equimolar concentration of all four catechol estrogens revealed the following order of product formation: 4-MeOE2>4-MeOE1>>2-MeOE2>2-MeOE1 >2-OH-3-MeOE1 >2-OH-3-MeOE2 (FIG. 10).

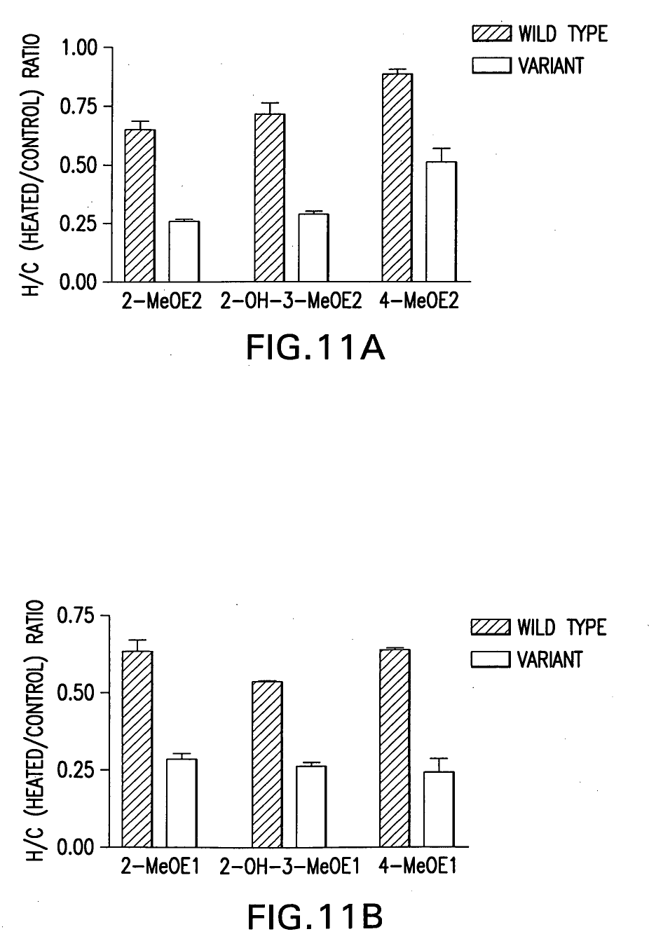

The experimental conditions used for the enzyme reaction (10 min at 37° C.) did not show a difference in recombinant wild-type and variant COMT activities. However, heat inactivation (15 min at 48° C.) prior to the enzyme reaction revealed a difference in thermal stability expressed as heated/control (H/C) ratio between wild-type and variant COMT. As shown in FIG. 11, the H/C ratio of the variant enzyme was significantly lower than the ratio of the wild-type enzyme, leading to two- to threefold lower levels of product formation after heating.

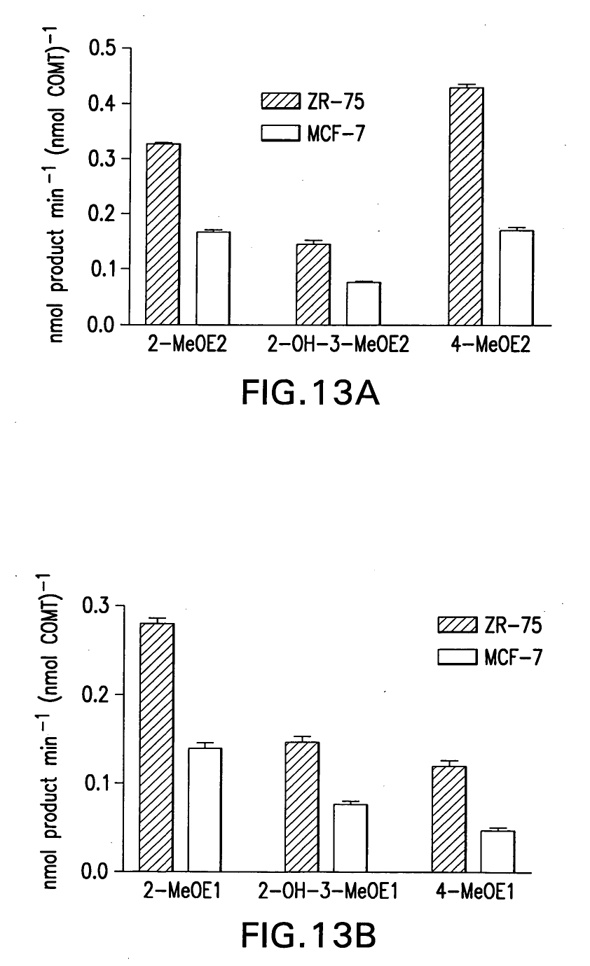

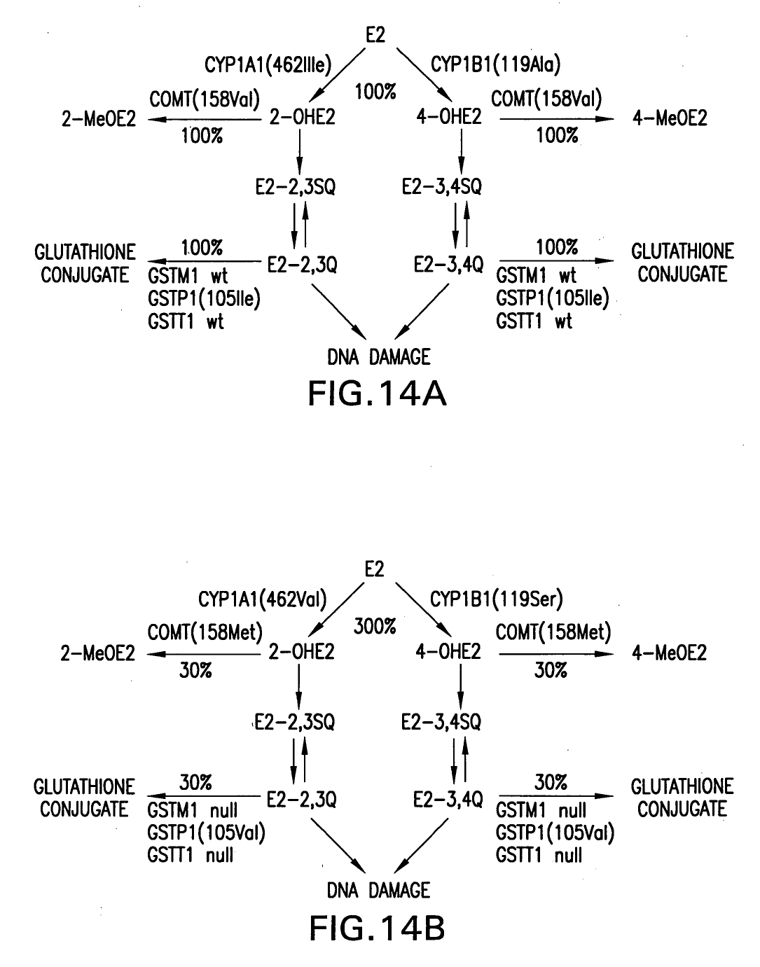

In order to directly compare the enzymatic activities of wild type COMT in ZR-75 cells and variant COMT in MCF-7 cells, an ICELISA was developed to quantify both enzymes as proteins. The H6 antibody, which was used for Western immunoblot, proved to be suboptimal for ICELISA. Therefore, another ScFv antibody was selected, designated C3, based on absorbance readings and an optimal dose-response curve for the concentration range 2.5-2500 ng/ml (FIG. 12). Wild type and variant COMT were indistinguishable by ICELISA. The concentration of COMT in ZR-75 and MCF-7 breast cancer cells was similar, i.e., 7.9±1.1 and 8.1±1.5 μg/mg cytosol protein. However, the enzymatic activity with respect to catechol estrogens differed significantly, as shown in FIG. 13. The variant COMT isoform in MCF-7 cells produced two- to threefold lower product levels than wild-type COMT in ZR-75 cells.

### EXAMPLE 5

### Genotype Determination

To determine whether variants of individual estrogen metabolizing genes affect breast cancer risk, and to determine whether the combination of estrogen metabolizing gene variants affects breast cancer risk, DNA is isolated from all samples using a DNA extraction kit (Stratagene, La Jolla, Calif.). The enzyme genotype analysis is carried out by PCR and restriction endonuclease digestion (Table 13). The specific primers and amplification conditions and the subsequent restriction endonuclease analysis for CYP1A1, CYP1B1 and GSTT1 were described previously (Bailey, 1998; Bailey, 1998). COMT is amplified with primers C1: (SEQ ID NO.:11) 5′-GCCGCCATCACCCAGCGGAT GGTGGATTTCGCTGTC and C2: (SEQ ID NO.: 12) 5′GTTTTCAGTGAACGTGGTGTG. The PCR analysis of COMT is improved by introducing a BspHI restriction site into the C1 primer (see underlined nucleotide) to reveal the methionine allele in codon 158 of the COMT gene. BspHI is a 6-base cutter with a single recognition site on the PCR product of the methionine allele and no site on the valine allele. Consequently digestion with BspHI yields bands of 160 bp for the Val/Val genotype, 160, 125 and 35 bp for the Val/Met genotype, and 125 and 35 bp for the Met/Met genotype. In contrast, the 4-base cutter NlaIII used in the original publication by Lachman et al. (Lachman, 1996) cleaves three sites on the methionine allele and two sites on the valine allele yielding relatively small restriction fragments of 67 and 71 bp, which are not easily distinguished from each other. For the analysis of GSTP1 polymorphisms in codons 105Ile→Val (exon 5) and 114Ala→Val (exon 6), primers and amplification conditions described by Watson et al. are used (Watson, 1998). However, a new primer P4 was designed to improve the detection of the 114Ala→Val polymorphism, which was based on the 4-base cutter Acil. Digestion with Acil yielded inconsistent results and required time-consuming and expensive DNA sequencing for confirmation (Watson, 1998). For this reason a Paul restriction site was introduced into the new P4 primer sequence 5′ (SEQ ID NO.: 19)-GTTGCCCGGGCAGTGCC TTCACATAGTCATCCTTGCGC (see underlined nucleotide). Paul digests the wild type 114 allele, but not the variant 114Val allele. Paul is a 6-base cutter allowing reliable restriction site recognition.

Standard quality control measures are employed for PCR testing. In particular, precautions to prevent cross contamination between samples are observed, which include physical separation of PCR studies and genomic DNA preparations, with separate pipetmen, plugged tips, storage areas and racks. Each PCR assay contains positive internal controls for the respective gene. Each PCR assay also has a negative control reaction tube containing all reagents except DNA template. The latter tube should be devoid of amplified products. In any case in which PCR products are visualized in the negative control tube, the results of that analysis are not accepted and the entire assay is repeated. In addition to the above control measures, random re-testing of approximately 5% of samples expecting 100% reproducibility based on previous experience is performed (Bailey, 1998; Bailey, 1998; Roodi, 1995; Yaich, 1992).

### EXAMPLE 6

### GSTM1 Genotype Associated with Cancer Risk

Subjects. The hospital-based case-control study group of 203 Caucasian and 59 African-American women with primary invasive breast cancer and their age-matched controls has been described previously (8, 122). Genomic DNA was extracted from tumor tissue or white blood cells. The DNA samples of one Caucasian control and five African-American cases had been depleted in previous studies, leaving 203 cases and 202 controls for the Caucasian population and 54 cases and 59 controls for the African-American study group.

PCR Analysis. Examples of primers for detection of the GSTM1 wt allele were M1, 5′-CTGCCCTACTTGATTGATGGG-3′ (SEQ ID NO:20) and M2,5′-CTGGATTGTAGCAGATCATGC-3′ (SEQ ID NO:21) (12). The primer pairs for detection of the GSTM1 null allele are listed herein, including nucleotide sequence, length, chromosome locus 1p13.3, and position on cosmid clones cgtm t1 (upstream primers) and cgtm 12 (downstream primers).

| | | |
|---|---|---|
| Primer Designation: | GSTM1-3 | (SEQ ID NO:22) |

| | |
|---|---|
| Sequence 5′---> 3′: | CCT GTT GAA GGA GCT TAT GCT GAA |
| Mer: | 24 |
| Locus | 1p13.3 |
| Cosmid clone cgtm1: | 15757 -15734 |

| | | |
|---|---|---|
| Primer Designation: | GSTM1-4 | (SEQ ID NO:23) |

| | |
|---|---|
| Sequence 5′---> 3′: | TTC TGA GGA CTG GAC TGA TGA TC |
| Mer: | 23 |
| Locus | 1p13.3 |
| cosmid clone cgtm 12: | 10238-10260 |

| | | |
|---|---|---|
| Primer Designation: | GSTM1-5 | (SEQ ID NO:24) |

| | |
|---|---|
| Sequence 5′---> 3′: | CTG ATG TAT CCA GCT GAA GCC TG |
| Mer: | 23 |
| Locus | 1p13.3 |
| Cosmid clone cgtm1: | 17438-17416 |

| | | |
|---|---|---|
| Primer Designation: | GSTM1-6 | (SEQ ID NO:25) |

| | |
|---|---|
| Sequence 5′---> 3′: | CAT TAG ACA GAA CGC ATG ACC AC |
| Mer: | 23 |
| Locus | 1p13.3 |
| cosmid clone cgtm 12: | 8579-8601 |

| | | |
|---|---|---|
| Primer Designation: | GSTM1-7 | (SEQ ID NO:26) |

| | |
|---|---|
| Sequence 5′---> 3′: | CTG GTC GAG AGC CTA CCA GGT GC |
| Mer: | 23 |
| Locus | 1p13.3 |
| Cosmid clone cgtm1: | 17595-17573 |

| | | |
|---|---|---|
| Primer Designation: | GSTM1-8 | (SEQ ID NO:27) |

| | |
|---|---|
| Sequence 5′---> 3′: | TGG GTA TGA TGA AGT TGA CCA C |
| Mer: | 22 |
| Locus | 1p13.3 |
| cosmid clone cgtm12: | 8402-8423 |

Amplification conditions for the wt allele were previously described (8). PCR of the null allele was carried out in a total volume of 50 μl containing 0.5-1.0 μg DNA by using the Expand 20 kb Plus PCR System as specified by the manufacturer (Roche Diagnostics, Indianapolis, Ind. Amplification conditions for primers identified as SEQ ID NOs:3-4 consisted of an initial denaturation step at 92° C. for 2 min, followed by 10 cycles of 92° C. for 10 s, 54° C. for 30 s, and 68° C. for 8 min, then by 29 cycles of 92° C. for 10 s, 54° C. for 30 s, and 68° C. for 8 min plus 10 s for each successive cycle, and final elongation at 68° C. for 10 min. The PCR protocol for primers identified as SEQ ID NOs:5-8 is identical, except the annealing temperature for SEQ ID NOs:5-8 is 60° C. Each PCR contained wt and null allele internal controls, and random samples were repeated to assure reproducibility. PCR products were electrophoresed in 0.5% SeaKem Gold agarose gel (Cambrex, East Rutherford, N.J.) and visualized by ethidium bromide staining.

Statistical Methods. Likelihood ratio tests of Hardy-Weinberg equilibrium for both cases and controls using the method of Elston and Forthofer were performed (39). Relative risks for breast cancer were estimated by odds-ratios and derived using logistic regression (32). These relative risks were adjusted for age by including age as a covariate in the regression models.

Results

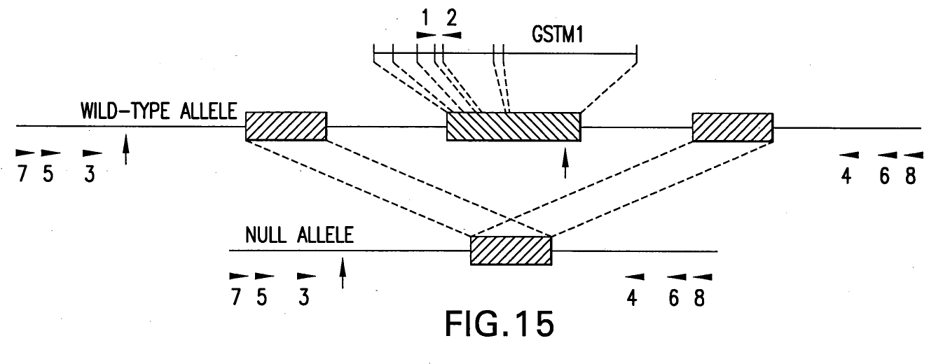

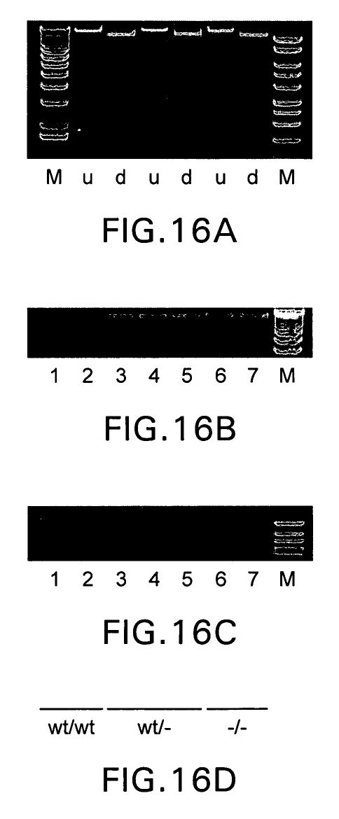

To develop a PCR-based strategy for the positive identification of the GSTM1 null allele, the GSTM1 gene locus at 1p13.3 was analyzed (FIG. 15). Initially, primers near the 4.2-kb repeat regions were designed, trying to utilize minor sequence differences between the upstream and downstream regions. However, the PCR reactions yielded ambiguous results. Primers in adjacent homologous regions also failed to produce unambiguous patterns. Finally, primers that annealed clearly outside the homologous regions flanking the GSTM1 gene were designed. This necessitated the use of long-range PCR amplification, which yielded a 14-kb product for the GSTM1 null allele (FIGS. 16A, B). To confirm the amplified sequence the restriction endonuclease SwaI, an 8-nucleotide cutter, with one recognition site in intron 7 and another upstream of the left repeat region, 1570 bp from primer 3 (SEQ ID NO:22) was selected (FIG. 15). Digestion of the 14-kb product yielded the expected two fragments of 12.4 and 1.6 kb (FIG. 16A). The expected PCR product of ˜30 kb for the wt allele could not be amplified due to its length.

All DNA samples were also analyzed by the established short-range PCR using the original primers within the GSTM1 gene to obtain a 273-bp product for the wild-type allele (FIG. 16C). The combined analysis of the two PCR reactions permitted positive identification of wild-type and null alleles resulting in GSTM1 genotyping of all individuals. Based on this approach, all samples were classified as +1+. +/− or −/− (FIG. 16D). Each PCR contained wild-type and null allele internal controls. The results of the short- and long-range PCR assays confirmed each other in every instance, i.e., samples lacking the wild-type allele always contained null allele and vice versa. As yet another control for the validity of the 14-kb PCR results and the integrity of the DNA samples, we developed an independent long-range PCR. We chose the ERa gene at 6q25.1 and designed primers to amplify a 14 kb fragment in exon and intron 4 of the ERa gene. In earlier studies we had shown that the ERa gene is present in all breast cancers including those that do not express the ERa protein (123, 108). We applied the assay to all +/+ samples and 50 randomly chosen +/− and −/− samples and obtained the ERa fragment in every instance (FIG. 16E).

The frequency of the GSTM1 wt and null alleles in the Caucasian control population was determined to be 0.225 and 0.775, respectively (Table 1). The distribution of homozygous and heterozygous individuals was consistent with Hardy-Weinberg equilibrium. There were 14 (6.9%) of 202 Caucasian controls with the wt/wt genotype but 37 (18.2%) among the 203 cases. Thus, the Caucasian cancer population showed a conspicuous deviation from the Hardy-Weinberg law with an excess of wt/wt individuals (P<0.0001). The frequency of the GSTM1 wt and null alleles in the African-American control population was 0.407 and 0.593, respectively (Table 1). Again, the distribution of homozygous and heterozygous individuals in the control population was consistent with Hardy-Weinberg equilibrium, whereas the cancer population deviated with an excess of wt/wt individuals (P=0.002).

Compared to Caucasian women with the −/− genotype, the relative risk of breast cancer for the +/− genotype was 0.83 (95% confidence interval (CI) 0.53-1.30; P=0.42) and for the +/+ genotype 2.82 (95% CI 1.45-5.49; P=0.002) (Table 2). There was no evidence for a gene-dose effect (P, trend=0.42). Among African-American women the +/+ genotype was observed in 13 (22.0%) of 59 controls compared to 18 (33.4%) of 54 cases, but the increased relative risk associated with the +/+ genotype did not reach significance (Table 2).

Discussion

The majority of polymorphisms affecting genes involved in carcinogen metabolism are single nucleotide polymorphisms (SNPs). Deletions are less common and the complete absence of a gene in the form of a null allele is rare. It is for this reason that the GSTM1−/− genotype has attracted so much attention and become the focus of over 500 publications in molecular epidemiology. While the detection of SNPs in DNA samples has become facile, the determination of associated functional disturbances in the protein products remains more difficult. However, even if a SNP is proven to affect protein function, the magnitude of the functional change in enzyme activity is usually less than 50% for the homozygous variant, e.g., glutathione S-transferase P1 (123). In contrast, the presence of 2, 1, or 0 GSTM1 alleles is associated with a clear-cut dosage effect resulting in 100, 50, or 0% enzyme activity. All preceding studies did not truly genotype GSTM1 but only identified −/− homozygosity and therefore could not separate the high and low GSH conjugator phenotypes associated with the +/+ and +/− genotypes. The positive identification of the wt and null alleles described here allowed definition of the +/+, +/−, and −/− genotypes and unambiguous assignment of high, low, and none conjugator phenotypes. Thus, the frequency of the GSTM1 wt and null alleles in the Caucasian and African-American populations can be determined, and the effect of the GSTM1 genotype on cancer risk can be assessed. The difference between the populations with the GSTM1 wt allele was defined as being nearly twice as common in African-American (0.407) than in Caucasian (0.225) women.

To examine the association of GSTM1 genotype with cancer risk, a breast cancer case-control study that had failed to show any effect of the GSTM1 −/− genotype was re-analyzed (8, 122). The GSTM1+/+ genotype occurred more frequently in Caucasian breast cancer patients and was associated with a significantly higher risk compared to the +/− and −/− genotypes. The association between the GSTM1+/+ genotype and elevated breast cancer risk was unexpected and requires an explanation, involving two factors (1) the substrate GSH and (2) the population genetics of the null deletion. Mammalian cells have evolved protective mechanisms such as GSH conjugation to minimize injurious events that result from toxic chemicals and normal oxidative products of cellular metabolism (108). GSH acts both as a nucleophilic scavenger of numerous compounds and their metabolites and as a substrate of the GSH-mediated destruction of hydroperoxides, including hydrogen peroxide, which is generated physiologically during mitochondrial oxygen consumption. Hydrogen peroxide, if not reduced, can lead to the formation of the highly reactive hydroxyl radical and cause damage to macromolecules including DNA. GSH depletion to about 20-30% of total glutathione levels can impair the conjugation defense against the toxic actions of such compounds and become detrimental to cellular processes (108). Without being bound by any specific mechanism, it may be that the combined conjugation activities of all GSTs leads to GSH depletion and thereby become counterproductive. Instead of protecting, the GSTs collectively may expose the cell to injurious effects such as oxidative DNA damage and associated mutagenic lesions. This may explain the high frequency of the GSTM1 −/− genotype. It seems that the deletion of the GSTM1 gene occurred not only with impunity but may actually have offered a survival advantage for the cell. It is unknown when the deletion of the human GSTM1 gene occurred, but it is interesting to note that the gene is found in African-American women at nearly twice the frequency as in their Caucasian counterparts.

The African-American study group also showed a higher frequency of the GSTM1+/+ genotype among cases than controls. Although the relative risk of breast cancer associated with the +/− and −/− genotypes was reduced in comparison to the +/+ genotype, the reduction was not significant. Possible reasons for the lack of significance are the smaller size of the study group and the different allele frequency in African-Americans. Whatever selection process favored the deletion of the GSTM1 gene in Caucasians may have magnified the difference in risk associated with the wt allele in relation to breast cancer. Besides GSTM1, there are other members of the GST superfamily that are expressed in breast tissue, such as GSTP1 and GSTA1 (138, 68). Interestingly, another GST family member, namely the GSTT1 gene at 22q11.2, can be deleted, resulting in the −/− genotype in 20% Caucasians and 47% Asians (43). The size and mechanism of the GSTT1 deletion have not been determined, and it is unknown whether the gene is expressed in breast tissue.

The present study involved a hospital-based breast cancer case-control population of Caucasian and African-American women. The same study population was re-analyzed to clarify the role of a single gene, GSTM1. The previous analysis, which was based on partial GSTM1 genotyping, failed to show an association with breast cancer (8, 122). The new analysis, based on complete genotyping, revealed an association of +/+ homozygosity with elevated risk in Caucasian women. In the present study, the results of the old and new PCR assays confirmed each other (i.e., samples lacking the wt allele always contained null allele and vice versa), making the possibility of erroneous genotyping even less likely. The combined identification of GSTM1 wt and null alleles is not only an analytical but also a conceptual advance based on sound biological reasoning. Regardless of the explanation underlying the association between the +/+ genotype and increased breast cancer risk, it will be useful to apply true GSTM1 genotyping to additional or previously analyzed groups of breast cancer and other malignancies to determine cancer risk.

Throughout this application, various publications are referenced. The disclosures of these publications in their entireties are hereby incorporated by reference into this application in order to more fully describe the state of the art to which this invention pertains.

### REFERENCES

1. Abul-Hajj Y J and Cisek, P L Catechol estrogen adducts. J Steroid Biochem. 31: 107-110, 1988.
2. Ali-Osman, F., Akande, O., Antoun, G., Mao, J., and Buolamwini, J. Molecular cloning, characterization, and expression in Escherichia coli of full-length cDNAs of three human glutathione S-transferase Pi gene variants. J Biol Chem, 15: 10004-10012, 1997.
3. Alpert, L. C., Schecter, R. L., Berry, D. A., Melnychuk, D., Peters, W. P., Caruso, J. A., Townsend, A. H., and Batist, G. Relation of glutathione S-transferase a and m isoforms to response to therapy in human breast cancer. Clin Cancer Res, 3: 661-667, 1997.
4. Ambrosone C B, Freudenheim J L, Graham S, et al. Cytochrome P4501A1 and glutathione S-transferase (M1) genetic polymorphisms and postmenopausal breast cancer risk. Cancer Res. 55:3483-3485, 1995.
5. Aoyama T, Korzekwa K, Nagata K, Gillette, J., Gelboin, H. V., and Gonzalez, F. J. Estradiol metabolism by complementary deoxyribonucleic acid-expressed human cytochrome P450s. Endocrinology 126: 3101-3106, 1990.
6. Axelrod J and Tomchick R. Enzymatic O-methylation of epinephrine and other catechols. J Biol. Chem. 233: 702-705, 1958.
7. Bailey L R, Roodi N, Dupont, W D, and Parl F F. Association of cytochrome P450 1B1 (CYP1B1) polymorphism with steroid receptor status in breast cancer [Erratum: Cancer Res 1999; 59:1388]. Cancer Res. 58: 5038-5041, 1998.
8. Bailey L R, Roodi N, Verrier C S, Yee C J, Dupont W D, Parl F F. Breast cancer and CYP1A1, GSTM1, and GSTT1 polymorphisms: Evidence of a lack of association in Caucasians and African Americans. Cancer Res. 58:65-70, 1998.
9. Ball P and Knuppen R. Catecholoestrogens (2- and 4-hydroxyoestrogens): chemistry, biogenesis, metabolism, occurrence and physiological significance. Acta Endocrin Suppl. 232: 1-127, 1980.
10. Ball P, Knuppen R, Haupt M., and Breuer, H. Interactions between estrogens and catecho lamines. 3. Studies on the methylation of catechol estrogens, catechol amines and other catechols by the catechol-O-methyl-transferases of human liver. J Clin Endocrin Metab. 34: 736-746, 1972.
11. Balta, G., Yuksek, N., Ozyurek, E., Ertem, U., Hicsonmez, G., Altay, C., and Gurgey, A. Characterization of MTHFR, GSTM1, GSTT1, GSTP1, and CYP1A1 genotypes in childhood acute leukemia. Am J Hematol, 73:154-160, 2003.
12. Barnea E R, MacLusky N J, and Naftolin F. Kinetics of catechol estrogen-estrogen receptor dissociation: a possible factor underlying differences in catechol estrogen biological activity. Steroids. 41: 643-656, 1983.
13. Bejjani B A, Lewis R A, Tomey, K F, Andersen, K. L., Dueker, D. K., Jabak, M., Astle, W. F., Otterud, B., Leppert, M., and Lupski, J. R. Mutations in CYP1B1, the gene for cytochrome P4501B1, are the predominant cause of primary congenital glaucoma in Saudia Arabia. Am J Hum Genet. 62: 325-333, 1998.
14. Berhane K, Widersten M, Engstrom A, Kozarich J. W, and Mannervik B. Detoxification of base propenals and other a,b-unsaturated aldehyde products of radical reactions and lipid peroxidation by human glutathione transferases. Proc Natl Acad. Sci. 91: 1480-1484, 1994.
15. Bertocci, B., Miggiano, V., Da Prada, M., Dembic, Z., Lahm, H. W., and Malherbe, P. Human catechol-O-methytransferase: Cloning and expression of the membrane-associated form. Proc Natl Acad. Sci. 88: 1416-1420, 1991.
16. Boudikova, B., Szumlanski, C., Maidak, B., and Weinshilboum, R. Human liver catechol-O-methyltransferase pharmacogenetics. Clin Pharmacol Ther. 48: 381-389, 1990.
17. Breslow N E, Day N E. Statistical Methods in Cancer Research, vol. 1. Lyon, France: IARC Publications, 1980.
18. Cantd-Paz E. Efficient and Accurate Parallel Genetic Algorithms. Kluwer Academic Publishers, Boston, 2000.
19. Cascorbi I, Brockmoller J, Roots I. A C4887A polymorphism in Exon 7 of human CYP1A1: population frequency, mutation linkages, and impact on lung cancer susceptibility. Cancer Res. 56:4965-4969, 1996.
20. Cavalieri, E. L., Stack, D. E., Devanesan, P. D., Todorvic, R., Dwivedy, I., Higginbotham, S., Johansson, S. L., Patil, K. D., Gross, M. L., Gooden, J. K., Ramanathan, R., and Cerny, R. L. Molecular origin of cancer: catechol estrogen-3,4-quinones as endogenous tumor initiators. Proc Natl Acad. Sci. 94: 10937-10942, 1997.
21. Chakravarti D, Pelling J C, Cavalieri E L, Rogan E G. Relating aromatic hydrocarbon-induced DNA adducts and c-H-ras mutations in mouse skin papillomas: the role of apurinic sites. Proc Natl Acad. Sci. 92:10422-10426, 1995.
22. Cheng, T., Christiani, D. C., Xu, X., Wain, J. C., Wiencke, J. K., and Kelsey, K. T. Glutathione S-transferase m genotype, diet and smoking as determinants of sister chromatid exchange frequency in lymphocytes. Cancer Epidemiol Biomarkers Prev, 4: 535-542, 1995.
23. Clemons M, Goss P Estrogen and the risk of breast cancer. New Engl J Med 344:276-285, 2001.
24. Collaborative Group on Hormonal Factors in Breast Cancer. Breast cancer and hormone replacement therapy: collaborative reanalysis of data from 51 epidemiological studies of 52 705 women with breast cancer and 108 411 women without breast cancer. Lancet 350:1047-1059, 1997.
25. Collaborative Group on Hormonal Factors in Breast Cancer. Breast cancer and hormonal contraceptives: collaborative reanalysis of individual data on 53 297 women with breast cancer and 100 239 women without breast cancer from 54 epidemiological studies. Lancet 347:1713-1727, 1996.
26. Concato J, Feinstein A R, Holford T R. The risk of determining risk with multivariablemodels. Ann Int Med 118:201-210, 1993.
27. Cosma, G., Crofts, F., Taioli, E., Toniolo, P., and Garte, S. Relationship between genotype and function of the human CYP1A1 gene. J Toxicol Environ Health 40: 309-316, 1993.
28. Coughlin, S. S. and Hall, I. J. Glutathione S-transferase polymorphisms and risk of ovarian cancer: a HUGE review. Genet Medicine, 4:250-257, 2002.
29. D'Amato, R. J., Lin, C. M., Flynn, E., Folkman, J., and Hamel, E. 2-Methoxyestradiol, an endogenous mammalian metabolite, inhibits tubulin polymerization by interacting at the colchicine site. Proc Natl Acad. Sci. 91: 3964-3968, 1994.
30. Deakin, M., Elder J., Hendrickse, C., Peckham, D., Baldwin, D., Pantin, C., Wild, N., Leopard, P., Bell, D. A., Jones, P., Duncan, H., Brannigan, K., Alldersea, J., Fryer, A. A., and Strange, R. C. Glutathione s-transferase GSTT1 genotypes and susceptibility to cancer: studies of interactions with GSTM1 in lung, oral, gastric and colorectal cancers. Carcinogenesis, 17: 881-884, 1996.
31. Dean, M., Carrington, M., Winkler, C., Huttley, G. A., Smith, M. W., Allikmets, R., Goedert, J. J., Buchbinder, S. P., Vittinghoff, E., Gomperts, E., Donfield, S., Vlahov, D., Kaslow, R., Saah, A., Rinaldo, C., Detels, R., and O'Brien, S. J. Genetic restriction of HIV-1 infection and progression to AIDS by a deletion allele of the CKR5 structural gene. Science, 273: 1856-1862, 1996.
32. Dupont, W. D. A Simple Introduction to the Analysis of Complex Data. Cambridge, U.K.: Cambridge University Press, 2002.
33. Dupont W D, Plummer W D. Power and sample size calculations for studies involving linear regression. Control Clin. Trials 19:589-601, 1998.
34. Dupont W D, Plummer W D. Power and sample size calculations: a review and computer program. Control Clin. Trials 11:116-128, 1990.
35. Dupont W D, Page D L, Rogers L W, Parl F F. Influence of exogenous estrogens, proliferative breast disease, and other variables on breast cancer risk. Cancer 63, No.5:948-957, 1989.
36. Dunning, A. M., Healey, C. S., Pharoah, P. D. P., Teare, M. D., Ponder, B. A. J., and Easton, D. F. A systematic review of genetic polymorphisms and breast cancer risk. Cancer Epidemiol Biomark Prev, 8: 843-854, 1999.
37. Dwivedy, I., Devanesan, P., Cremonesi, P., Rogan, E., and Cavalieri, E. Synthesisand characterization of estrogen 2,3- and 3,4-quinones. Comparison of DNA adducts formed by the quinones versus horseradish peroxidase-activated catechol estrogens. Chem Res Toxicol. 5: 828-833, 1992.
38. Elexpuru-Camiruaga, J., Buxton, N., Kandula, V., Dias, P. S., Campbell, D., McIntosh, J., Broome, J., Jones, P., Inskip, A., and Alldersea, J. Susceptibility to astrocytoma and meningioma: influence of allelism at glutathione S-transferase (GSTT1 and GSTM1) and cytochrome P-450 (CYP2D6) loci. Cancer Res, 55: 4237-4239, 1995.
39. Elston, R. C. and Forthofer, R. Testing for Hardy-Weinberg equilibrium in small samples. Biometrics, 33: 536-542, 1977.
40. Floyd R A. The role of 8-hydroxyguanine in carcinogenesis. Carcinogenesis 11:1447-1450, 1990.
41. Fotsis, T., Zhang, Y., Pepper, M. S., Adlercreutz, H., Montesano, R., Nawroth, P. P., and Schweigerer, L. The endogenous oestrogen metabolite 2-methoxyoestradiol inhibits angiogenesis and suppresses tumour growth. Nature 368: 237-239, 1994.
42. Frankel W N, Schork N J. Who's afraid of epistasis? Nat Genet 14: 371-373, 1993.
43. Garte, S., Gaspari, L., Alexandrie, A. K., Ambrosone, C., Autrup, H., Aurup, J. L., Baranova, H., Bathum, L., Benhamou, S., Boffetta, P., Bouchardy, C., Breskvar, K., Brockmoller, J., and et al. Metabolic gene polymorphism frequencies in control populations. Cancer Epidemiol Biomark Prev, 10: 1239-1248, 2001.
44. Geisler, S. A. and Olshan, A. F. GSTM1, GSTT1, and the risk of squamous cell carcinoma of the head and neck: a mini-HuGE review. Am J Epidemiol, 154: 95-105, 2001.
45. Gillam, E. M., Guo, Z., Ueng, Y. F., Yamazaki, H., Cock, I., Reilly, P. E., Hooper, W. D., and Guengerich, F. P. Expression of cytochrome P450 3A5 in Escherichia coli: effects of 5′ modification, purification, spectral characterization, reconstitution conditions, and catalytic activities. Arch Biochem Biophys. 317: 374-384, 1995.
46. Grossman, M. H., Creveling, C. R., Rybczynski, R., Braverman, M., Isersky, C., and Breakefield, X. O. Soluble and particulate forms of rat catechol-O-ethyltransferase distinguished by gel electrophoresis and immune fixation. J. Neurochem. 44: 421-432, 1985.
47. Guengerich, F. P., Gillam, E. M., and Shimada, T. New applications of bacterial systems to problems in toxicology. Crit Rev Toxicol. 26: 551-583, 1996.
48. Guengerich, F. P. Oxidation-reduction properties of rat liver cytochromes P-450 and NADPH-cytochrome P-450 reductase related to catalysis in reconstituted systems. Biochemistry. 22: 2811-2820, 1983.
49. Han, X. and Liehr, J. G. Microsome-mediated 8-hydroxylation of guanine bases of DNA by steroid estrogens: correlation of DNA damage by free radicals with metabolic activation to quinones. Carcinogenesis 16: 2571-2574, 1995
50. Han X, Liehr JG. DNA single-strand breaks in kidneys of Syrian hamsters treated with steroidal estrogens: hormone-induced free radical damage preceding renal malignancy. Carcinogenesis 15:997-1000, 1994.
51. Hanna, I. H., Dawling, S., Roodi, N., Guengerich, F. P., and Parl, F. F. Cytochrome P450 1B1 (CYP1B1) pharmacogenetics: association of polymorphisms with functional differences in estrogen hydroxylation activity. Cancer Res. 60: 3440-3444, 2000.
52. Hanna, I. H., Teiber, J. F., Kokones, K. L., and Hollenberg, P. F. Role of the alanine at position 363 of cytochrome P450 2B2 in influencing the NADPH- and hydroperoxide-supported activities. Arch Biochem Biophys. 350: 324-332, 1998.
53. Harris J R, Lippman M E, Veronesi U, Willett W. Breast cancer. New Engl J Med 327:319-328, 1992.
54. Hayashi S, Watanabe J, Nakachi K, Kawajiri K. Genetic linkage of lung cancer-associated Msp1 polymorphisms with amino acid replacement in the heme binding region of the human cytochrome P4501A1 gene. J. Biochem. 110:407-411, 1991.
55. Hayes, C. L., Spink, D. C., Spink, B. C., Cao, J. Q., Walker, N. J., and Sutter, T. R. 17b-estradiol hydroxylation catalyzed by human cytochrome P450 1B1. Proc Natl Acad. Sci. 93: 9776-9781, 1996.
56. Hayes, J. D. and Pulford, D. J. The glutathione S-transferase supergene family: Regulation of GST and the contribution of the isoenzymes to cancer chemoprotection and drug resistance. Crit Rev Biochem Mol Biol, 30: 445-600, 1995.
57. Heagerty, A., Smith, A., Engish, J., Lear, J., Perkins, W., Bowers, B., Jones, P., Gilford, J., Alldersea, J., Fryer, A., and Strange, R. C. Susceptibility to multiple cutaneous basal cell carcinomas: significant interactions between glutathione S-transferase GSTM1 genotypes, skin type and male gender. Br J Cancer, 73:44-48, 1996.
58. Helzlsouer K J, Selmin O, Huang H Y, et al. Association between glutathione S-transferase M1, P1, and T1 genetic polymorphisms and development of breast cancer. J Natl Cancer Inst. 90:512-518, 1998
59. Hirvonen, A., Pelin, K., Tammilehto, L., Karjalainen, A., Mattson, K., and Linnainmaa, K. Inherited GSTM1 and NAT2 defects as concurrent risk modifiers in asbestos-related human malignant mesothelioma. Cancer Res, 55:2981-2983, 1995.
60. Hosmer D W, Lemeshow S. Applied Logistic Regression. John Wiley & Sons Inc., New York, 2000.
61. Huang, P., Feng, L., Oldham, E. A., Keating, M. J., and Plunkett, W. Superoxide dismutase as a target for the selective killing of cancer cells. Nature 407: 390-395, 2000.
62. Huang, Z., Fasco, M. J., Figge, H. L., Keyomarsi, K., and Kaminsky, L. S. Expression of cytochromes P450 in human breast tissue and tumors. Drug Metab Disposition 24: 899-905, 1996.
63. Imoto, S., Mitani, F., Enomoto, K., Fujiwara, K., Ikeda, T., Kitajima, M., and Ishimura, Y. Influence of estrogen metabolism on proliferation of human breast cancer. Breast Cancer Res Treat. 42: 57-64, 1997.
64. Ishibe N, Hankinson S E, Colditz G A, et al. Cigarette smoking, cytochrome P450 1A1 polymorphisms, and breast cancer risk in the Nurses' Health Study. Cancer Res. 58:667-671, 1998.
65. Iverson, S. L., Shen, L., Anlar, N., and Bolton, J. L. Bioactivation of estrone and its catechol metabolites to quinoid-glutathione conjugates in rat liver microsomes. Chem Res Toxicol. 9: 492-499, 1996.
66. Jeffery, D. R. and Roth, J. A. Characterization of membrane-bound and soluble catechol-O-methyltransferase from human frontal cortex. J. Neurochem. 42: 826-832, 1984.
67. Kawajiri K, Nakachi K, Imai K, Watanabe J, Hayashi S. The CYP1A1 gene and cancer susceptibility. Crit. Rev. Oncol-Hemat. 14:77-87, 1993.
68. Kelley, M. K., Engqvist-Goldstein, A., Montali, J. A., Wheatley, J. B., Schmidt, J., D. E., and Kauvar, L. M. Variability of glutathione S-transferase isoenzyme patterns in matched normal and cancer human breast tissue. Biochem J, 304: 843-848, 1994.
69. Kelsey K T, Hankinson S E, Colditz G A, et al. Glutathione S-transferase class mu deletion polymorphism and breast cancer: results from prevalent versus incident cases. Cancer Epidemiol Biomarkers Prev 6:511-515, 1997.
70. Kelsey J L, Gammon M D, John E M. Reproductive and hormonal risk factors. Epidemiol Rev 15:36-47, 1993.
71. Kelsey J L, Berkowitz G S. Breast cancer epidemiology. Cancer Res 48:5615-5623, 1988.
72. Kempf, A. C., Zanger, U. M., and Meyer, U. A. Truncated human P450 2D6: expression in Eschericia coli, Ni2+-chelate affinity purification, and characterization of solubility and aggregation. Arch Biochem Biophys. 321: 277-288, 1995.
73. Kerridge, I., Lincz, L., Scorgie, F., Hickey, D., Granter, N., and Spencer, A. Association between xenobiotic gene polymorphisms and non-Hodgkin's lymphoma risk. Br J Haematol, 118:477-481, 2002.
74. Klauber, N., Parangi, S., Flynn, E., Hamel, E., and D'Amato, R. J. Inhibition of angiogenesis and breast cancer in mice by the microtubule inhibitors 2-ethoxyestradiol and taxol. Cancer Res. 57: 81-86, 1997.
75. Lachman, H. M., Papolos, D. F., Saito, T., Yu, Y., Szumlanski, C. L., and Weinshilboum, R. M. Human catechol-O-methyltransferase pharmacogenetics: description of a functional polymorphism and its potential application to neuropsychiatric disorders. Pharmacogenetics 6: 243-250, 1996.
76. Lafuente, A., Pujol, F., Carretero, P., Villa, J. P., and Cuchi, A. Human glutathione S-transferase mu (GST mu) deficiency as a marker for the susceptibility to bladder and larynx cancer among smokers. Cancer Lett, 68:49-54, 1993.
77. Landi, M. T., Bertazzi, P. A., Shields, P. G., Clark, G., Lucier, G. W., Garte, S. J., Cosma, G., and Caporaso, N. E. Association between CYP1A1 genotype, mRNA expression and enzymatic activity in humans. Pharmacogenetics 4: 242-246, 1994.
78. Lavigne, J. A., Helzlsouer, K. J., Huang, H., Strickland, P. T., Bell, D. A., Selmin, O., Watson, M. A., Hoffman, S., Comstock, G. W., and Yager, J. D. An association between the allele coding for a low activity variant of catechol-O-methyltransferase and the risk for breast cancer. Cancer Res. 57: 5493-5497, 1997.
79. Li W, Reich J. A complete enumeration and classification of two-locus disease models. Hum Hered 50:334-349, 2000.
80. Li, J. J. and Li, S. A. Estrogen carcinogenesis in Syrian hamster tissues: role of metabolism. Fed Proc. 46: 1858-1863, 1987.
81. Liehr, J. G. Is estradiol a genotoxic mutagenic carcinogen? Endocrine Rev. 21: 40-54, 2000.
82. Liehr, J. G. and Ricci, M. J. 4-Hydroxylation of estrogens as marker of human mammary tumors. Proc Natl Acad. Sci. 93: 3294-3296, 1996.
83. Liehr, J. G. Genotoxic effects of estrogens. Mutation Res. 238: 269-276, 1990.
84. Liehr, J. G. and Roy, D. Free radical generation by redox cycling of estrogens. Free Radical Biol Med. 8: 415-423, 1990.
85. Liehr, J. G., Fang, W. F., Sirbasku, D. A., and Ari-Ulubelen, A. Carcinogenicity of catechol estrogens in Syrian hamsters. J Steroid Biochem. 24: 353-356, 1986.
86. Liehr, J. G., Ulubelen, A. A., and Strobel, H. W. Cytochrome P-450-mediated redox cycling of estrogens. J Biol. Chem. 261: 16865-16870, 1986.
87. Lin, H. J., Probst-Hensh, N. M., Ingles, S. A., Han, C. Y., Lin, B. K., Lee, D. B., Frankl, H. D., Lee, E. R. Longnecker, M. P., and Haile, R. W. Glutathione transferase (GSTM1) null genotype, smoking, and prevalence of colorectal adenomas. Cancer Res, 55:1224-1226, 1995.
88. London, S. J., Daly, A. K., Cooper, J., Navidi, W. C., Carpenter, C. L., and Idle, J. R. Polymorphism of glutathione S-transferase M1 and lung cancer risk among African-Americans and Caucasians in Los Angeles County, California. J Natl Cancer Inst, 87:1246-1253, 1995.
89. Longuemaux, S., Delomenie, C., Gallou, C., Mejean, A., Vincent-Viry, M., Bouvier, R., Droz, D., Krishnamoorty, R., Galteau, M. M., Junien, C., Beroud, C., and Dupret, J. M. Candidate genetic modifiers of individual susceptibility to renal cell carcinoma: a study of polymorphic human xenobiotic-metabolizing enzymes. Cancer Res, 59:2903-2908, 1999.
90. Lotta, T., Vidgren, J., Tilgmann, C., Ulmanen, I., Melen, K., Julkunen, I., and Taskinen, J. Kinetics of human soluble and membrane-bound catechol O-methyltransferase: a revised mechanism and description of the thermolabile variant of the enzyme. Biochemistry 34: 4202-4210, 1995.
91. Lottering, M. L., Haag, M., and Seegers, J. C. Effects of 17b-estradiol metabolites on cell cycle events in MCF-7 cells. Cancer Res. 52: 5926-5932, 1992.
92. MacDonald P C, Edman C D, Hemsell D L, Porter J C, Siiteri P K. Effect of obesity on conversion of plasma androstenedione to estrone in postmenopausal women with and without endometrial cancer. Am J Obstet Gynecol. 130:448-455, 1978.
93. Malherbe, P., Bertocci, B., Caspers, P., Zurcher, G., and Da Prada, M. Expression of functional membrane-bound and soluble catechol-O-methyltransferase in Escherichia coli and a mammalian cell line. J. Neurochem. 58: 1782-1789, 1992.
94. Mannervik, B., Awasthi, Y. C., Board, P. G., Hayes, J. D., Di Ilio, C., Ketterer, B., Listowsky, I., Morgenstern, R., Muramatsu, M., Pearson, W. R., Pickett, C. B., Sato, K., Widerstein, M., and Wolf, C. R. Nomenclature for human glutathione transferases. Biochem J, 282: 305-308, 1992.
95. Matsui, A., Ikeda, T., Enomoto, K., Nakashima, H., Omae, K., Watanabe, M., Hibi, T., and Kitajima, M. Progression of human breast cancers to the metastatic state is linked to genotypes of catechol-O-methyltransferase. Cancer Lett. 150: 23-31, 1999.
96. Michnovicz, J. J., Hershcopf, R. J., Naganuma, H., Bradlow, H. L., and Fishman, J. Increased 2-hydroxylation of estradiol as a possible mechanism for the anti-estrogenic effect of cigarette smoking. New England J. Med. 315: 1305-1309, 1986.
97. Millikan, R. C., Pittman, G. S., Tse, C. K. J., Duell, E., Newman, B., Savitz, D., Moorman, P. G., Boissy, R. J., and Bell, D. A. Catechol-O-methyltransferase and breast cancer risk. Carcinogenesis 19: 1943-1947, 1998.
98. Moore J W, Key T J, Bulbrook R D, et al. Sex hormone binding globulin and risk factors for breast cancer in a population of normal women who had never used exogenoussex hormones. Br J Cancer 56:661-666, 1987.
99. Mukhopadhyay, T. and Roth, J. A. Superinduction of wild-type p53 protein after 2methoxyestradiol treatment of Ad5p53-transduced cells induces tumor cell apoptosis. Oncogene 17: 241-246, 1998.
100. Nandi S, Guzman R C, Yang J. Hormones and mammary carcinogenesis in mice, rats, and humans: a unifying hypothesis, Proc Natl Acad. Sci. 92:3650-3657, 1995.
101. Nebert, D. W. Elevated estrogen 16alpha-hydroxylase activity: is this a genotoxic ornongenotoxic biomarker in human breast cancer risk? J Natl Cancer Inst. 85: 1888-1891, 1993.
102. Nelson M, Kardia S L R, Ferrell R E, Sing C F. A combinatorial partitioning method to identify multilocus genotypic partitions that predict quantitative trait variation. Genome Res 11:458-470, 2001.
103. Newbold, R. R. and Liehr, J. G. Induction of uterine adenocarcinoma in CD-1 mice by catechol estrogens. Cancer Res. 60: 235-237, 2000.
104. Nutter, L. M., Wu, Y. Y., Ngo, E. O., Sierra, E. E., Gutierrez, P. L., and Abul-Hajj, Y. J. An o-quinone form of estrogen produces free radicals in human breast cancer cells: correlation with DNA damage. Chem Res Toxicol. 7: 23-28, 1994.
105. Omura, T. and Sato, R. The carbon monoxide-binding pigment of liver microsomes. I. evidence for its hemoprotein nature. J Biol. Chem. 239: 2370-2378, 1964.
106. Osborne, M. P., Bradlow, H. L., Wong, G. Y. C., and Telang, N. T. Upregulaton of estradiol C16alpha-hydroxylation in human breast tissue: a potential biomarker of breast cancer risk. J Natl Cancer Inst. 85: 1917-1920, 1993.
107. Paradiso A, Vetrugno M G, Capuano G, et al. Expression of GST-mu transferase in breast cancer patients and healthy controls. Int J Biol Markers 9:219-223, 1994.
108. Parl, F. F., Multiple mechanisms of estrogen receptor gene repression contribute to ER-negative breast cancer. Pharmacogenomics J, 3:251-253, 2003.
109. Parl, F. F. Estrogens, Estrogen Receptor and Breast Cancer. Amsterdam: IOS Press, 2000.
110. Peduzzi P, Concato J, Kemper E, Holford T R, Feinstein A R. A simulation study of the number of events per variable in logistic regression analysis. J Clin Epidemiol 49:1373-1379, 1996.
111. Perera, F. P. Molecular epidemiology: insights into cancer susceptibility, risk assessment, and prevention. J Natl Cancer Inst. 88: 496-509, 1996.
112. Perrett, C. W., Clayton, R. N., Pistorello, M., Boscaro, M., Scanarini, M., Bates, A. S., Buckley, N., Jones, P., Fryer, A. A., and Gilford, J. GSTM1 and CYP2D6 genotype frequencies in patients with pituitary tumours: effects on p53, ras and gsp. Carcinogenesis, 16:1643-1645, 1995.
113. Persson I, Johansson I, Ingelman-Sundberg M. In vitro kinetics of two human CYP1A1 variant enzymes suggested to be associated with interindividual differences in cancer susceptibility. Biochem. Biophys. Res. Comm. 231:227-230, 1997.
114. Petersen, D. D., McKinney, C. E., Ikeya, K., Smith, H. H., Bale, A. E., McBride, O. W., and Nebert, D. W. Human CYP1A1 gene: cosegregation of the enzyme inducibility phenotype and an RFLP. Am J Hum Genet. 48: 720-725, 1991.
115. Pope, T., Embelton, J., and Mernaugh, R. L. Building antibody gene repertories. In: J. McCafferty, D. Chiswell, and H. Hoogenboom (eds.), Antibody Engineering: A Practical Approach, pp. 1-40. New York: IRL Press, 1996.
116. Potischman N, Swanson C A, Siiteri P, Hoover R N. Reversal of relation between body mass and endogenous estrogen concentrations with menopausal status. J Natl Cancer Inst. 88:756-758, 1996.
117. Rebbeck T R. Molecular epidemiology of the human glutathione S-transferase genotypes GSTM1 and GSTT1 in cancer susceptibility. Cancer Epidemiol. Biomarkers Prev 6:733-743, 1997.
118. Rebbeck T, Resvold E A, Duggan D J, Zhang J, Buetow K H. Genetics of CYP1A1: Coamplification of specific alleles by polymerase chain reaction and association with breast cancer. Cancer Epidem Biomarkers Prev. 3:511-514, 1994.
119. Rebbeck, T. R., Walker, A. H., Jaffe, J. M., White, D. L., Wein, A. J., and Malkowicz, S. B. Glutathione S-transferase-mu (GSTM1) and -theta (GST1) genotypes in the etiology of prostate cancer. Cancer Epidemiol Biomark Prevent, 8:283-287, 1999.
120. Reed, D. J. Glutathione: toxicological implications. Annu Rev Pharmacol Toxicol, 30: 603-631, 1990.
121. Ripley BD. Pattern Recognition and Neural Networks. Cambridge University Press, Cambridge, 1996.
122. Ritchie, M. D., Hahn, L. W., Roodi, N., Bailey, L. R., Dupont, W. D., Parl, F. F., and Moore, J. H. Multifactor-dimensionality reduction reveals high-order interactions among estrogen-metabolism genes in sporadic breast cancer. Am J Hum Genet, 69: 138-147, 2001.
123. Roodi, N., Bailey, L. R., Kao, W. Y., Verrier, C. S., Yee, C. J., Dupont, W. D., and Parl, F. F., Estrogen receptor gene analysis in estrogen receptor-positive and receptor-negative primary breast cancer. J Natl Cancer Inst, 87:446-451, 1995.
124. Roy, D., Weisz, J., and Liehr, J. G. The O-methylation of 4-hydroxyestradiol is inhibited by 2-hydroxyestradiol: implications for estrogen induced carcinogenesis. Carcinogenesis 11: 459-462, 1990.
125. Rothman, K. J. and Greenland, S. Modern Epidemiology, 2nd edition. Philadelphia: Lippincott-Raven, 1998.
126. Scanlon, P. D., Raymond, F. A., and Weinshilboum, R. M. Catechol-O-methyltransferase: thermolabile enzyme in erythrocytes of subjects homozygous for allele for low activity. Science 203: 63-65, 1979.
127. Schlichting C D, Pigliucci M. Phenotypic Evolution: A Reaction Norm Perspective. Sinauer Associates, Inc., Sunderland, 1998.
128. Schutze, N., Vollmer, G., and Knuppen, R. Catecholestrogens are agonists of estrogen receptor dependent gene expression in MCF-7 cells. J Steroid Biochem Mol. Biol. 48: 453-461, 1994.
129. Schutze, N., Vollmer, G., Tiemann, I., Geiger, M., and Knuppen, R. Catecholestrogens are MCF-7 cell estrogen receptor agonists. J Steroid Biochem Mol. Biol. 46: 781-789, 1993.
130. Seidegard J, Vorachek W R, Pero R W, Pearson W R. Hereditary differences in the expression of the human glutathione transferase active on trans-stilbene oxide are due to a gene deletion. Proc Natl Acad. Sci. 85:7293-7297, 1988.
131. Shanley, S. M., Chenevix-Trench, G., Palmer, J., and Hayward, N. Glutathione S-transferase GSTM1 null genotype is not overrepresented in Australian patients with nevoid basal cell carcinoma syndrome or sporadic melanoma. Carcinogenesis, 16:2003-2004, 1995.
132. Shimada, T., Watanabe, J., Kawajiri, K., Sutter, T. R., Guengerich, F. P., Gillam, E. M. J., and Inoue, K. Catalytic properties of polymorphic human cytochrome P450 1B1 variants. Carcinogenesis 20: 1607-1613, 1999.
133. Shimada, T., Wunsch, R. W., Hanna, I. H., Sutter, T. R., Guengerich, F. P., and Gillam, E. M. J. Recombinant human cytochrome P450 1B1 expression in Escherichia coli. Arch Biochem Biophys. 357: 111-120, 1998.
134. Shimada, T., Hayes, C. L., Yamazaki, H., Amin, S., Hecht, S. S., Guengerich, F. P., and Sutter, T. R. Activation of chemically diverse procarcinogens by human cytochrome P-450 1B1. Cancer Res. 56: 2979-2984, 1996.
135. Spink, D. C., Hayes, C. L., Young, N. R., Christou, M., Sutter, T. R., Jefcoate, C. R., and Gierthy, J. F. The effects of 2,3,7,8-tetrachlorodibenzo-p-dioxin on estrogen metabolism in MCF-7 breast cancer cells: evidence for induction of a novel 17 beta-estradiol 4-hydroxylase. J Steroid Biochem Mol. Biol. 51: 251-258, 1994.
136. Spink, D. C., Eugster, H., Lincoln, D. W. I., Schuetz, J. D., Schuetz, E. G., Johnson, J. A., Kaminsky, L. S., and Gierthy, J. F. 17 beta-estradiol hydroxylation catalyzed by human cytochrome P450 1A1: a comparison of the activities induced by 2,3,7,8-tetrachlorodibenzo-p-dioxin in MCF-7 cells with those from heterologous expression of the cDNA. Arch Biochem Biophys. 293: 342-348, 1992.
137. Stack, D. E., Cavalieri, E. L., and Rogan, E. G. Catecholestrogens procarcinogens: depurinating adducts and tumor initiation. Adv Pharmacol. 42: 833-836, 1998.
138. Stephens, J. C., Reich, D. E., Goldstein, D. B., Shin, H. D., Smith, M. W., Carrington, M., Winkler, C., Huttley, G. A., Allikmets, R., Schriml, L., Gerrard, B., Malasky, M., Ramos, M. D., Morlot, S., Tzetis, M., Oddoux, C., Di Diovane, F. S., Nasoulas, G., Chandler, D., Aseev, M., Hanson, M., Kalaydjeva, L., Glavac, D., Gasparini, P., Dean, M., and et al. Dating of the origin of the CCR5-Delta32 AIDS-resistance allele by the coalescence of haplotypes. Am J Hum Genet, 62: 1507-1515, 1998.
139. Strange, R. C., Spiteri, M. A., Ramachandran, S., and Fryer, A. Glutathione-S-transferase family of enzymes. Mutat Res, 482: 21-26, 2001.
140. Stuart A, Ord J K. Kendall's Advanced Theory of Statistics, vol. 2. London: Edward Arnold, 1991.
141. Sutter, T. R., Tang, Y. M., Hayes, C. L., Wo, Y. P., Jabs, E. W., Li, X., Yin, H., Cody, C. W., and Greenlee, W. F. Complete cDNA sequence of a human dioxin-inducible mRNA identifies a new gene subfamily of cytochrome P450 that maps to chromosome 2. J Biol. Chem. 269: 13092-13099, 1994.
142. Syvanen, A. C., Tilgmann, C., Rinne, J., and Ulmanen, I. Genetic polymorphism of catechol-O-methyltransferase (COMT): correlation of genotype with individual variation of S-COMT activity and comparison of the allele frequencies in the normal population and parkinsonian patients in Finland. Pharmacogenetics 7: 65-71, 1997.
143. Tabakovic, K., Gleason, W. B., Ojala, W. H., and Abul-Hajj, Y. J. Oxidative transformation of 2-hydroxyestrone. Stability and reactivity of 2,3-estrone quinone and its relationship to estrogen carcinogenicity. Chem Res Toxicol. 9: 860-865, 1996.
144. Tenhunen, J., Salminen, M., Lundstrom, K., Kiviluoto, T., Savolainen, R., and Ulmanen, I. Genomic organization of the human catechol O-methyltransferase gene and its expression from two distinct promoters. Eur J. Biochem. 223: 1049-1059, 1994.
145. Thompson, P. A., Shields, P. G., Freudenheim, J. L., Stone, A., Vena, J. E., Marshall, J. R., Graham, S., Laughlin, R., Nemoto, T., Kadlubar, F. F., and Ambrosone, C. B. Genetic polymorphisms in catechol-O-methyltransferase, menopausal status, and breast cancer risk. Cancer Res. 58: 2107-2110, 1998.
146. Tsutsui, T., Tamura, Y., Hagiwara, M., Miyachi, T., Hikiba, H., Kubo, C., and Barrett, J. C. Induction of mammalian cell transformation and genotoxicity by 2-methoxyestradiol, an endogenous metabolite of estrogen. Carcinogenesis 21: 735-740, 2000.
147. Ulmanen, I., Peranen, J., Tenhunen, J., Tilgmann, C., Karhunen, T., Panula, P., Bernasconi, L., Aubry, J. P., and Lundstrom, K. Expression and intracellular localization of catechol O-methyltransferase in transfected mammalian cells. Eur J. Biochem. 243: 452-459, 1997.
148. Ulmanen, I. and Lundstrom, K. Cell-free synthesis of rat and human catechol O-methyltransferase. Eur J. Biochem. 202: 1013-1020, 1991.
149. Van Aswegen, C. H., Purdy, R. H., and Wittliff, J. L. Binding of 2-hydroxyestradiol and 4-hydroxyestradiol to estrogen receptors from human breast cancers. J Steroid Biochem. 32: 485-492, 1989.
150. Vidgren, J., Svensson, L. A., and Liljas, A. Crystal structure of catechol O-methyltransferase. Nature 368: 354-357, 1994.
151. Vistisen, K., Prime, H., Okkels, H., Valentin, S., Loft, S., Olsen, J. H., and Poulsen, H. E. Genotype and phenotype of glutathione S-transferase-mu in testicular cancer patients. Pharmacogenetics, 7:21-25, 1997.
152. Wade M J. Epistasis as a Genetic Constraint within Populations and an Accelerant of Adaptive Divergence among Them. In: Wade M, Brodie III B, Wolf J (eds) Epistasis and Evolutionary Process. Oxford University Press, 2000.
153. Warwick, A. P., Redman, C. W., Jones, P. W., Fryer, A. A., Gilford, J., Alldersea, J., and Strange, R. C. Progression of cervical intraepithelial neoplasia to cervical cancer: interactions of cytochrome P450 CYP2D6 EM and glutathione S-transferase GSTM1 null genotypes and cigarette smoking. Br J. Cancer, 70:704-708, 1994.
154. Waxman, D. J., Lapenson, D. P., Aoyama, T., Gelboin, H. V., Gonzalez, F. J., and Korzekwa, K. Steroid hormone hydroxylase specificities of eleven cDNA-expressed human cytochrome P450s. Arch Biochem Biophys. 290: 160-166, 1991.
155. Wilson A F, Bailey-Wilson J E, Pugh E W, Sorant A J M. The Genometric Analysis Simulation Program (G.A.S.P.): A software tool for testing and investigating methods in statistical genetics. Am J Hum Genet 59:A193, 1996.
156. Xu, S. J., Wang, Y. P., Roe, B., and Pearson, W. R. Characterization of the human class mu glutathione S-transferase gene cluster and the GSTM1 deletion. J Biol Chem, 273: 3517-3527, 1998.
157. Yager, J. D. and Liehr, J. G. Molecular mechanisms of estrogen carcinogenesis. Annu Rev Pharmacol Toxicol. 36: 203-232, 1996.
158. Yong L C, Brown C C, Schatzkin A, Schairer C. Prospective study of relative weight and risk of breast cancer: the Breast Cancer Detection Demonstration Project follow-up study 1979 to 1987-1989. Am J Epidemiol 143:985-995, 1996.
159. Yu, M. W., Gladek-Yarborough, A., Chiamprasert, S., Santella, R. M., Liaw, Y. F., and Chen, C. J. Cytochrome P450 2E1 and glutathione S-transferase M1 polymorphisms and susceptibility to hepatocellular carcinoma. Gastroenterology, 109:1266-1273, 1995.
160. Zhang Z, Fasco M J, Huang L, Guengerich F P, Kaminsky L S. Characterization of purified human recombinant cytochrome P4501A1-Ile 462 and -Val 462 Assessment of a role for the rare allele in carcinogenesis. Cancer Res. 56:3926-3933, 1996.
161. Zhong S, Wyllie A H, Barnes D, Wold C R, Spurr N K. Relationship between the GSTM1 genetic polymorphism and susceptibility to bladder, breast, and colon cancer. Carcinogenesis 14:1821-1824, 1993.
162. Zhu, B. T. and Conney, A. H. Is 2-methoxyestradiol an endogenous estrogen metabolite that inhibits mammary carcinogenesis? Cancer Res. 58: 2269-2277, 1998.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

TABLE 1
Enzyme Genotype Analysis by PCR and Restriction Endonuclease Digestion

| Enzyme | Polymorphism | Primers | Endonuclease | Genotype | | |
|---|---|---|---|---|---|---|
| CYP1A1 | T6235C creates new MspI site | A3, A4 | MspI/SphI | T/T | T/C | C/C |
| m1 | in 3′ untranslated region | | | | | |
| M2 | A4889G results in Ile462Val and may | A1, A2 | BsrD1 | Ile/Ile | Ile/Val | Val/Val |
| | increase enzymatic activity | | | | | |
| M4 | C4887A results in Thr461Asn with | A1, A4 | BsaI | Thr/Thr | Thr/Asn | Asn/Asn |
| | unknown functional effect | | | | | |
| CYP1B1 | G1294C results in Val432Leu with | B1, B2 | Eco57I | Val/Val | Val/Leu | Leu/Leu |
| M1 | unknown functional effect | | | | | |
| M2 | A1358G results in Asn453Ser with | B1, B2 | Cac8I | Asn/Asn | Asn/Ser | Ser/Ser |
| | unknown functional effect | | | | | |
| COMT | G1947A results in Val158Met with | C1, C2 | BspHI | Val/Val | Val/Met | Met/Met |
| | 3- to 4-fold lower activity | | | | | |
| GSTT1 | Null deletion results in loss of enzyme | T1, T2 | | wild type | Null | |

TABLE 2
Genotypes of 207 Controls and 207 age-matched Breast Cancer Patients

| | | Cases | Controls | Total |
|---|---|---|---|---|
| Enzyme | Genotype | n (%) | n (%) | n (%) |
| CYP1A1 m1 | T/T | 159 (76.8) | 173 (83.6) | 332 (80.2) |
| | T/C | 42 (20.3) | 29 (14.0) | 71 (17.1) |
| | C/C | 6 (2.9) | 5 (2.4) | 11 (2.7) |
| m2 | Ile/Ile | 187 (90.3) | 191 (92.3) | 378 (91.3) |
| | Ile/Val | 20 (9.7) | 16 (7.7) | 36 (8.7) |
| m4 | Thr/Thr | 189 (91.3) | 193 (93.2) | 382 (92.3) |
| | Thr/Asp | 16 (7.7) | 13 (6.3) | 29 (7.0) |
| | Asp/Asp | 2 (1.0) | 1 (0.5) | 3 (0.7) |
| CYP1B1 m1 | Leu/Leu | 61 (29.5) | 59 (28.5) | 120 (29.0) |
| | Val/Leu | 111 (53.6) | 113 (54.6) | 224 (54.1) |
| | Val/Val | 35 (16.9) | 35 (16.9) | 70 (16.9) |
| m2 | Asn/Asn | 143 (69.1) | 141 (68.1) | 284 (68.6) |
| | Asn/Ser | 57 (27.5) | 61 (29.5) | 118 (28.5) |
| | Ser/Ser | 7 (3.4) | 5 (2.4) | 12 (2.9) |
| COMT | Val/Val | 58 (28.0) | 51 (24.6) | 109 (26.3) |
| | Val/Met | 97 (46.9) | 107 (51.7) | 204 (49.3) |
| | Met/Met | 52 (25.1) | 49 (23.7) | 101 (24.4) |
| GSTT1 | wild type/heterozygous | 147 (71.0) | 152 (73.4) | 299 (72.2) |
| | null | 60 (29.0) | 55 (26.6) | 115 (27.8) |

TABLE 3
Mean Age and BMI of Cases and Controls

| | Cases | Controls |
|---|---|---|
| Premenopausal | | |
| No. Subjects | 58 | 56 |
| Age | 41.3 ± 6.2 | 39.7 ± 7.8 |
| BMI | 26.5 ± 6.2 | 27.9 ± 8.7 |
| Postmenopausal | | |
| No. of Subjects | 149 | 151 |
| Age | 64.1 ± 11.9 | 64.3 ± 12.2 |
| BMI | 25.4 ± 4.9b | 26.5 ± 5.9c |

a mean ± SD
bbased on 147 cases
cbased on 146 controls

TABLE 4
Association between Genotypes and Postmenopausal Breast Cancer Risk stratified by BMI

| | | BMI £25.5 kg/m2 | | | BMI > 25.5 kg/m2 | | |
|---|---|---|---|---|---|---|---|
| Gene | Genotype | Cases | Controls | OR (95% CI) | Cases | Controls | OR (95% CI) |
| CYP1A1 m1 | T/T | 63 | 57 | 1.0 | 47 | 65 | 1.0 |
| | T/C or C/C | 21 | 9 | 2.13 (0.90-5.05) | 16 | 15 | 1.47 (0.66-3.26) |
| M2 | Ile/Ile | 74 | 60 | 1.0 | 56 | 75 | 1.0 |
| | Ile/Val | 10 | 6 | 1.35 (0.47-3.94) | 7 | 5 | 1.86 (0.56-6.18) |
| M4 | Thr/Thr | 80 | 61 | 1.0 | 56 | 74 | 1.0 |
| | Thr/Asp or Asp/Asp | 4 | 5 | 0.62 (0.16-2.44) | 7 | 6 | 1.53 (0.49-4.82) |
| CYP1B1 m1 | Leu/Leu | 30 | 15 | 1.0 | 12 | 26 | 1.0 |
| | Val/Leu | 39 | 37 | 0.53 (0.25-1.13) | 41 | 41 | 2.15 (0.96-4.85) |
| | Val/Val | 15 | 14 | 0.54 (0.21-1.39) | 10 | 13 | 1.65 (0.57-4.84) |
| M2 | Asn/Asn | 60 | 42 | 1.0 | 46 | 55 | 1.0 |
| | Asn/Ser or Ser/Ser | 24 | 24 | 0.70 (0.35-1.40) | 17 | 25 | 0.81 (0.39-1.68) |
| COMT | Val/Val | 29 | 8 | 1.0 | 14 | 27 | 1.0 |
| | Val/Met | 37 | 42 | 0.24 (0.10-0.60) | 32 | 35 | 1.76 (0.79-3.94) |
| | Met/Met | 18 | 16 | 0.31 (0.11-0.88) | 17 | 18 | 1.80 (0.71-4.57) |
| | Val/Met or Met/Met | 55 | 58 | 0.26 (0.11-0.62) | 49 | 53 | 1.78 (0.84-3.78) |
| GSTT1 | wild type or heterozygous | 59 | 58 | 1.0 | 43 | 57 | 1.0 |
| | Null | 25 | 8 | 3.13 (1.30-7.54) | 20 | 23 | 1.16 (0.56-2.38) |

TABLE 5
Association between Combined Genotypes and Postmenopausal Breast Cancer Risk stratified by BMI

| | BMI £ 25.5 kg/m2 | | | BMI > 25.5 kg/m2 | | |
|---|---|---|---|---|---|---|
| Combined Genotypes | Cases | Controls | OR (95% C.I.) | Cases | Controls | OR (95% C.I.) |
| CYP1B1 m1 Leu/Leu and | 14 | 9 | 1.0 | 7 | 9 | 1.0 |
| CYP1B1 m2 Asn/Asn | | | | | | |
| CYP1B1 m1 Leu/Val or Val/Val and CYP1B1 m2 | 8 | 18 | 0.29 (0.09-0.96) | 12 | 8 | 1.90 (0.48-7.6) |
| Asn/Ser or Ser/Ser | | | | | | |
| CYP1B1 m1 Leu/Leu and | 10 | 4 | 1.0 | 2 | | |
| COMT Val/Val | | | | | 12 | 1.0 |
| CYP1B1 m1 Leu/Val or Val/Val and COMT | 35 | 47 | 0.33 (0.09-1.1) | 39 | 39 | 6.07 (1.3-29) |
| Val/Met or Met/Met | | | | | | |
| CYP1B1 m2 Asn/Asn and | 24 | 4 | 1.0 | 11 | 14 | 1.0 |
| COMT Val/Val | | | | | | |
| CYP1B1 m2 Asn/Ser or Ser/Ser and | 19 | 20 | 0.16 (0.05-0.56) | 14 | 12 | 1.94 (0.56-6.4) |
| COMT Val/Met or Met/Met | | | | | | |

TABLE 6
Association between COMT Genotypes and Postmenopausal Breast Cancer Risk
stratified by BMI [based on cumulative data from present study and studies by Lavigne
et al. Lavigne, 1997, Thompson et al. Thompson, 1998 #41, and Millikan et al. 1998

| | | Lean BMI | | | Obese BMI | | |
|---|---|---|---|---|---|---|---|
| Gene | Genotype | Cases | Controls | RR (95% CI) | Cases | Controls | RR (95% CI) |
| COMT | Val/Val | 105 | 69 | 1.0 | 99 | 119 | 1.0 |
| | Val/Met or Met/Met | 233 | 269 | 0.57 (0.40-0.81) | 205 | 224 | 1.10 (0.79-1.53) |

TABLE 7
CYP1B1 Gene Polymorphisms and Plasmids used for Recombinant CYP1B1 Expression

| Codons | 48Arg ® Gly | 119Ala ® Ser | 432Val ® Leu | 453Asn ® Ser |
|---|---|---|---|---|
| Plasmids | | | | |
| wild type a | Arg | Ala | Val | Asn |
| variant 1 | Glyb | Ala | Val | Asn |
| variant 2 | Arg | Ser | Val | Asn |
| variant 3 | Arg | Ala | Leu | Asn |
| variant 4 | Arg | Ala | Val | Ser |
| variant 5 | Gly | Ser | Leu | Ser |

^{a}Based on published amino acid sequence (44)
^{b}Amino acid substitutions are indicated in bold letters

TABLE 8
Estradiol hydroxylation activities of CYP1B1 wild type and variants^{a}

| | 2-OH-Estradiol | | | 4-OH-Estradiol | | | 4-OH-E2/ | 16a-OH-Estradiol | | |
|---|---|---|---|---|---|---|---|---|---|---|
| | | | kcat/Km | | | kcat/Km | 2-OH-E2 | Km | kcat | kcat/Km |
| CYP1B1 | Km (mM) | kcat (min-1) | (mM-1 min-1) | Km (mM) | kcat (min-1) | (mM-1 min-1) | kcat/Km | (mM) | (min-1) | (mM-1 min-1) |
| Wild type | 34 ± 4 | 1.9 ± 0.1 | 55 ± 7 | 40 ± 8 | 4.4 ± 0.4 | 110 ± 24 | 2.0 ± 0.5 | 39 ± 6 | 0.30 ± 0.02 | 7.6 ± 1.3 |
| Variant 1 | 29 ± 5 | 3.2 ± 0.2 | 110 ± 20 | 19 ± 2 | 6.0 ± 0.2 | 320 ± 35 | 3.0 ± 0.6 | 65 ± 9 | 0.56 ± 0.04 | 8.6 ± 1.3 |
| Variant 2 | 18 ± 2 | 2.3 ± 0.1 | 130 ± 15 | 10 ± 1 | 3.8 ± 0.1 | 370 ± 38 | 3.0 ± 0.5 | 41 ± 6 | 0.34 ± 0.02 | 8.4 ± 1.3 |
| Variant 3 | 21 ± 2 | 2.2 ± 0.1 | 110 ± 12 | 11 ± 1 | 3.7 ± 0.1 | 330 ± 31 | 3.0 ± 0.4 | 19 ± 1 | 0.31 ± 0.01 | 16 ± 1.0 |
| Variant 4 | 39 ± 5 | 2.8 ± 0.2 | 71 ± 10 | 17 ± 2 | 4.5 ± 0.3 | 270 ± 36 | 3.8 ± 0.8 | 29 ± 3 | 0.39 ± 0.02 | 14 ± 1.6 |
| Variant 5 | 29 ± 3 | 2.5 ± 0.1 | 86 ± 10 | 15 ± 2 | 4.4 ± 0.1 | 290 ± 39 | 3.3 ± 0.6 | 43 ± 7 | 0.40 ± 0.03 | 9.4 ± 1.7 |

^{a}Data represent means ± standard errors of duplicate assays. Hydroxylation reactions were conducted as described in Materials and Methods.

TABLE 9
Enzyme Genotype Analysis by PCR and Restriction Endonuclease Digestion

| | | | | | | Genotype | | |
|---|---|---|---|---|---|---|---|---|
| | | | | | EndonucleaseError! | Frequency | | |
| | | Polymorphism | | | Bookmark | (%)^{a} | | |
| Enzyme | | Nucleotide | Codon | Primers^{Error! Bookmark not defined.} | not defined. | w/w | w/p | p/p |
| CYP1A1 | m2 | 4887C → A | 461Thr → Asn | A1, A4^{c} | BsaI | 92 | 7 | 1 |
| | m4 | 4889A → G | 462Ile → Val | A1, A2^{c} | BsrDI | 92 | 8 | 0 |
| | m1 | T6235T → C | 3′ UTR^{b} | A3, A4^{c} | MspI | 82 | 15 | 3 |
| CYP1B1 | | 143C → G | 48Arg → Gly | B1, B2^{d} | RsrII | 51 | 40 | 9 |
| | | 355G → T | 119Ala → Ser | B1, B2^{d} | NgoMIV | 51 | 40 | 9 |
| | | 1294G → C | 432Val → Leu | B3, B4^{d} | Eco57I | 12 | 58 | 30 |
| | | 1358A → G | 453Asn → Ser | B3, B4^{d} | Cac8I | 68 | 30 | 2 |
| COMT | | 1947G → A | 158Val → Met | C1, C2 | BspHI | 25 | 51 | 24 |

| | | | | | | |
|---|---|---|---|---|---|---|
| GSTT1 | Deletion | Loss of enzyme | T1, T2^{c} | — | 79^{e} | 21 |

^{a}w = wild type allele; p = polymorphic allele;
^{b}UTR = untranslated region
^{c}Bailey et al., 1998a;
^{d}Bailey et al., 1998b;
^{e}either w/w/ or w/p genotype

TABLE 10
Summary of Simulation Results

| | | |
|---|---|---|
| | CV² | Prediction |
| Number of | Consistency | Error |

| Model¹ | Loci | Mean | SE³ | Mean | SE |
|---|---|---|---|---|---|
| 2 | 2 | 9.86 | 0.08 | 14.99 | 0.24 |
| | 3 | 7.41 | 0.21 | 15.58 | 0.26 |
| | 4 | 6.01 | 0.22 | 16.49 | 0.29 |
| | 5 | 5.56 | 0.24 | 19.03 | 0.38 |
| | 6 | 6.52 | 0.34 | 23.23 | 0.53 |
| | 7 | 6.94 | 0.26 | 24.49 | 0.62 |
| | 8 | 7.90 | 0.29 | 25.02 | 0.73 |
| | 9 | 8.03 | 0.23 | 25.40 | 0.73 |
| 3 | 2 | 9.20 | 0.17 | 21.91 | 0.33 |
| | 3 | 10.00 | 0.00 | 12.00 | 0.22 |
| | 4 | 9.27 | 0.13 | 12.37 | 0.24 |
| | 5 | 6.28 | 0.21 | 13.90 | 0.28 |
| | 6 | 5.86 | 0.25 | 15.57 | 0.32 |
| | 7 | 6.26 | 0.29 | 17.75 | 0.43 |
| | 8 | 7.68 | 0.28 | 19.39 | 0.47 |
| | 9 | 7.99 | 0.25 | 19.93 | 0.50 |
| 4 | 2 | 8.40 | 0.26 | 19.15 | 0.35 |
| | 3 | 8.79 | 0.20 | 10.20 | 0.23 |
| | 4 | 10.00 | 0.00 | 5.68 | 0.17 |
| | 5 | 9.32 | 0.12 | 6.02 | 0.19 |
| | 6 | 7.74 | 0.16 | 6.88 | 0.22 |
| | 7 | 7.01 | 0.22 | 7.73 | 0.26 |
| | 8 | 7.04 | 0.24 | 8.64 | 0.31 |
| | 9 | 7.79 | 0.24 | 9.46 | 0.34 |
| 5 | 2 | 9.01 | 0.20 | 15.33 | 0.28 |
| | 3 | 8.37 | 0.25 | 8.54 | 0.24 |
| | 4 | 8.16 | 0.25 | 5.17 | 0.20 |
| | 5 | 9.99 | 0.01 | 2.95 | 0.11 |
| | 6 | 9.52 | 0.12 | 3.17 | 0.14 |
| | 7 | 9.13 | 0.16 | 3.66 | 0.17 |
| | 8 | 8.74 | 0.17 | 4.17 | 0.19 |
| | 9 | 9.00 | 0.14 | 4.60 | 0.18 |

¹Number of epistatic genes in each simulation model.
²CV: Cross Validation
³SE: Standard Error

TABLE 11
Summary of Breast Cancer Data Results

| | CV¹ | |
|---|---|---|
| Number of Loci | Consistency | Prediction Error |
| 2 | 7.00 | 51.06 |
| 3 | 4.17 | 51.35 |
| 4 | 9.80* | 46.73 |
| 5 | 4.71 | 50.26 |
| 6 | 5.00 | 48.61 |
| 7 | 8.60 | 47.15 |
| 8 | 8.20 | 52.55 |
| 9 | 7.10 | 53.40 |

¹CV: Cross Validation
*p < 0.001

TABLE 12
Kinetic Parameters for COMT-Mediated Catechol Estrogen Metabolism

| Products | Kₘ | k_{cat} | k_{cat}/Kₘ | Hill Coefficient |
|---|---|---|---|---|
| 2-MeOE2 | 108 ± 9 | 6.8 ± 0.4 | 63 ± 6 | n.a.* |
| 2-OH-3-MeOE2 | 51 ± 5 | 1.5 ± 0.1 | 29 ± 3 | n.a. |
| 4-MeOE2 | 24 ± 3 | 3.4 ± 0.2 | 142 ± 20 | 1.6 ± 0.2 |
| 2-MeOE1 | 74 ± 8 | 3.3 ± 0.2 | 45 ± 6 | n.a. |
| 2-OH-3-MeOE1 | 73 ± 16 | 2.8 ± 0.4 | 38 ± 10 | n.a. |
| 4-MeOE1 | 53 ± 6 | 6.7 ± 0.4 | 126 ± 16 | 2.0 ± 0.4 |

*not applicable, the best fit was to a Michaelis-Menten curve

TABLE 13
Enzyme Genotype Analysis by PCR and Restriction Endonuclease Digestion

| | | | | | Genotype | | |
|---|---|---|---|---|---|---|---|
| | Polymorphism | | | | Frequency^{a} | | |
| Enzyme | Nucleotide | Codon | Primers | Endonuclease | w/w | w/p | p/p |
| CYP1A1 | 4887C → A | 461Thr → Asn | A1, A4^{c} | BsaI | 92 | 7 | 1 |
| | 4889A → G | 462Ile → Val | A1, A2^{c} | BsrDI | 92 | 8 | 0 |
| | T6235T → C | 3′ UTR^{b} | A3, A4^{c} | MspI | 82 | 15 | 3 |
| CYP1B1 | 143C → G | 48Arg → Gly | B1, B2^{d} | RsrII | 51 | 40 | 9 |
| | 355G → T | 119Ala → Ser | B1, B2^{d} | NgoMIV | 51 | 40 | 9 |
| | 1294G → C | 432Val → Leu | B3, B4^{d} | Eco57I | 12 | 58 | 30 |
| | 1358A → G | 453Asn → Ser | B3, B4^{d} | Cac8I | 68 | 30 | 2 |
| COMT | 1947G → A | Val158Val → | C1, C2 | BspHI | 25 | 51 | 24 |
| | | Met | | | | | |
| | | | | | | | 43 |
| GSTP1 | A → G | 105Ile → Val | P1, P2^{e} | Alw26I | 42 | 51 | 7 |
| | C → T | 114Ala → Val | P3^{e}, P4 | Paul | 82 | 18 | 0 |

| | | | | | | |
|---|---|---|---|---|---|---|
| GSTT1 | Deletion | Loss of enzyme | T1, T2^{c} | — | 79 | 21 |

^{a}w = wild type allele; p = polymorphic allele;
^{b}UTR = untranslated region
^{c}Bailey et al., 1998(Bailey, 1998);
^{d}Bailey et al., 1998(Bailey, 1998);
^{e}Watson et al., 1998(Watson, 1998 #3438)

TABLE 14
Distribution of GSTM1 genotypes in breast cancer cases and controls

| | Genotype | | | |
|---|---|---|---|---|
| | Caucasian | | African-American | |
| | Cases | | | Controls |
| | (n = 203) | Controls (n = 202) | Cases (n = 54) | (n = 59) |
| wt/wt | 37 (18.2)a | 14 (6.9) | 18 (33.4) | 13 (22.0) |
| wt/— | 49 (24.1) | 63 (31.2) | 16 (29.6) | 22 (37.3) |
| —/— | 117 (57.7) | 125 (61.9) | 20 (37.0) | 24 (40.7) |

aNumber of individuals followed by percentage in parentheses

TABLE 15
Relative risk of breast cancer based on GSTM1 genotypes

| | Caucasian | | | African-American | | |
|---|---|---|---|---|---|---|
| | Relative | | | Relative | | |
| Genotype | Risk^{a} | 95% C.I.^{b} | P value | Risk | 95% C.I. | P value |
| −/− | 1.00^{c} | | | 1.00 | | |
| +/− | 0.83 | 0.53-1.30 | 0.42 | 0.88 | 0.36-2.10 | 0.77 |
| +/+ | 2.82 | 1.45-5.49 | 0.002 | 1.66 | 0.66-4.20 | 0.28 |

^{a}adjusted for age;
^{b}95% confidence interval;
^{c}denominator for following relative risks

1. A method for identifying a subject having an increased risk of developing cancer, comprising determining the allele or alleles of the subject's GSTM1 gene, whereby a subject being homozygous for the wild-type allele or a subject being heterozygous for the wild-type and null alleles is identified as having an increased risk of developing cancer.

2. The method of claim 1, wherein the subject is homozygous for the wild-type allele.

3. The method of claim 1, wherein the subject is heterozygous for the wild-type allele and the null allele.

4. The method of claim 1, wherein the subject is a mammal.

5. The method of claim 4, wherein the mammal is human.

6. The method of claim 1, wherein the cancer is breast cancer.

7. A method for identifying a subject having a decreased risk of developing cancer, comprising determining the allele or alleles of the subject's GSTM1 gene, whereby a subject having an allele of the GSTM1 gene which is correlated with a decreased risk of developing cancer and which comprises a homozygous null allele is identified as having a decreased risk of developing cancer.

8. The method of claim 7, wherein the subject is a mammal.

9. The method of claim 8, wherein the mammal is human.

10. The method of claim 7, wherein the cancer is breast cancer.

11. A diagnostic kit for determining the presence in a subject of an allele of the gene encoding GSTM1 that is correlated with an increased risk of developing cancer, comprising means for distinguishing a homozygous wild-type subject from a heterozygous wild-type/null subject.

12. The kit of claim 11, wherein the identifying means comprises a first nucleic acid primer pair having a nucleic acid with the sequence identified as SEQ ID NO:20 and a nucleic acid with the sequence identified as SEQ ID NO:21, and a second nucleic acid primer pair selected from the group of primer pairs consisting of a nucleic acid primer pair having a nucleic acid with the sequence identified as SEQ ID NO:22 and a nucleic acid with the sequence identified as SEQ ID NO:23; a nucleic acid primer pair having a nucleic acid with the sequence identified as SEQ ID NO:24 and a nucleic acid with the sequence identified as SEQ ID NO:25; and a nucleic acid primer pair having a nucleic acid with the sequence identified as SEQ ID NO:26 and a nucleic acid with the sequence identified as SEQ ID NO:27.

13. The kit of claim 11, wherein the cancer is breast cancer.

14. A diagnostic kit for determining the presence in a subject of a homozygous null allele of the gene encoding GSTM1 that is correlated with a decreased risk of developing cancer, comprising means for identifying the allele of the subject's GSTM1 gene in a biological sample from the subject, wherein the identifying means comprises a nucleic acid primer pair selected from the group of primer pairs having a nucleic acid with the sequence identified as SEQ ID NO:22 and a nucleic acid with the sequence identified as SEQ ID NO:23; a nucleic acid primer pair having a nucleic acid with the sequence identified as SEQ ID NO:24 and a nucleic acid with the sequence identified as SEQ ID NO:25; and a nucleic acid primer pair having a nucleic acid with the sequence identified as SEQ ID NO:26 and a nucleic acid with the sequence identified as SEQ ID NO:27.

15. The kit of claim 14, wherein the cancer is breast cancer.

16. An isolated nucleic acid having the sequence identified as SEQ ID NO:22.

17. An isolated nucleic acid having the sequence identified as SEQ ID NO:23.

18. An isolated nucleic acid having the sequence identified as SEQ ID NO:24.

19. An isolated nucleic acid having the sequence identified as SEQ ID NO:25.

20. An isolated nucleic acid having the sequence identified as SEQ ID NO:26.

21. An isolated nucleic acid having the sequence identified as SEQ ID NO:27.

22. A pair of primers, wherein the primers are from about 15 to about 35 nucleotides in length, and wherein one of the primers has a nucleotide sequence specific for cosmid clone cg/m1 from about nucleotide 15734 to about nucleotide 17595, and another primer has a nucleotide sequence specific for cosmid clone cg/m12 from about nucleotide 8402 to about nucleotide 10260.

23. The pair of primers of claim 22, wherein the pair is selected from the group of pairs of primers consisting of a nucleic acid with the sequence identified as SEQ ID NO:20 and a nucleic acid with the sequence identified as SEQ ID NO:21; a pair of primers consisting of a nucleic acid with the sequence identified as SEQ ID NO:22 and a nucleic acid with the sequence identified as SEQ ID NO:23; a pair of primers consisting of a nucleic acid with the sequence identified as SEQ ID NO:24 and a nucleic acid with the sequence identified as SEQ ID NO:25; and a pair of primers consisting of a nucleic acid with the sequence identified as SEQ ID NO:26 and a nucleic acid with the sequence identified as SEQ ID NO:27.


SEQUENCE LISTING (29 sequences)

SEQ ID NO: 1 - DNA, 20 bp, Artificial Sequence
  Description of Artificial Sequence; note = synthetic construct
ggctgagcaatctgacccta

SEQ ID NO: 2 - DNA, 21 bp, Artificial Sequence
  Description of Artificial Sequence; note = synthetic construct
ggccccaactactcagaggct

SEQ ID NO: 3 - DNA, 21 bp, Artificial Sequence
  Description of Artificial Sequence; note = synthetic construct
gaaaggctgggtccaccctct

SEQ ID NO: 4 - DNA, 30 bp, Artificial Sequence
  Description of Artificial Sequence; note = synthetic construct
ccaggaagagaaagacctcccagcgggcca

SEQ ID NO: 5 - DNA, 21 bp, Artificial Sequence
  Description of Artificial Sequence; note = synthetic construct
gtggtttttgtcaaccagtgg

SEQ ID NO: 6 - DNA, 37 bp, Artificial Sequence
  Description of Artificial Sequence; note = synthetic construct
gcccactgaaaaaatcatcactctgctggtcaggtgc

SEQ ID NO: 7 - DNA, 21 bp, Artificial Sequence
  Description of Artificial Sequence; note = synthetic construct
ccttggccgctaaacccgctg

SEQ ID NO: 8 - DNA, 20 bp, Artificial Sequence
  Description of Artificial Sequence; note = synthetic construct
ctggcgcgtgaagaagttgc

SEQ ID NO: 9 - DNA, 23 bp, Artificial Sequence
  Description of Artificial Sequence; note = synthetic construct
ttccttactggtcctcacatctc

SEQ ID NO: 10 - DNA, 20 bp, Artificial Sequence
  Description of Artificial Sequence; note = synthetic construct
tcaccggatcatggccagca

SEQ ID NO: 11 - DNA, 36 bp, Artificial Sequence
  Description of Artificial Sequence; note = synthetic construct
gccgccatcacccagcggatggtggatttcgctgtc

SEQ ID NO: 12 - DNA, 21 bp, Artificial Sequence
  Description of Artificial Sequence; note = synthetic construct
gttttcagtgaacgtggtgtg

SEQ ID NO: 13 - DNA, 39 bp, Artificial Sequence
  Description of Artificial Sequence; note = synthetic construct
cgggatccctcctgtcggtgctggccactgtgcatgtgg

SEQ ID NO: 14 - DNA, 32 bp, Artificial Sequence
  Description of Artificial Sequence; note = synthetic construct
ggggtaccttattggcaagtttccttggcttg

SEQ ID NO: 15 - DNA, 29 bp, Artificial Sequence
  Description of Artificial Sequence; note = synthetic construct
caacggaggcggcagctcgggtccgcgcc

SEQ ID NO: 16 - DNA, 29 bp, Artificial Sequence
  Description of Artificial Sequence; note = synthetic construct
ggcgcggacccgagctgccgcctccgttg

SEQ ID NO: 17 - DNA, 27 bp, Artificial Sequence
  Description of Artificial Sequence; note = synthetic construct
cgaccggccgtccttcgcctccttccg

SEQ ID NO: 18 - DNA, 27 bp, Artificial Sequence
  Description of Artificial Sequence; note = synthetic construct
cggaagcaggcgaaggacggccggtcg

SEQ ID NO: 19 - DNA, 38 bp, Artificial Sequence
  Description of Artificial Sequence; note = synthetic construct
gttgcccgggcagtgccttcacatagtcatccttgcgc

SEQ ID NO: 20 - DNA, 21 bp, Artificial Sequence
  Description of Artificial Sequence; note = synthetic construct
ctgccctacttgattgatggg

SEQ ID NO: 21 - DNA, 21 bp, Artificial Sequence
  Description of Artificial Sequence; note = synthetic construct
ctggattgtagcagatcatgc

SEQ ID NO: 22 - DNA, 24 bp, Artificial Sequence
  Description of Artificial Sequence; note = synthetic construct
cctgttgaaggagcttatgctgaa

SEQ ID NO: 23 - DNA, 23 bp, Artificial Sequence
  Description of Artificial Sequence; note = synthetic construct
ttctgaggactggactgatgatc

SEQ ID NO: 24 - DNA, 23 bp, Artificial Sequence
  Description of Artificial Sequence; note = synthetic construct
ctgatgtatccagctgaagcctg

SEQ ID NO: 25 - DNA, 23 bp, Artificial Sequence
  Description of Artificial Sequence; note = synthetic construct
cattagacagaacgcatgaccac

SEQ ID NO: 26 - DNA, 23 bp, Artificial Sequence
  Description of Artificial Sequence; note = synthetic construct
ctggtccagagcctaccaggtgc

SEQ ID NO: 27 - DNA, 22 bp, Artificial Sequence
  Description of Artificial Sequence; note = synthetic construct
tgggtatgatgaagttgaccac

SEQ ID NO: 28 - DNA, 38703 bp, Artificial Sequence
  Description of Artificial Sequence; note = synthetic construct
tagaatctgaccactcagtcccacatcccagggttcagggacaaagaatcccgctgagggctctggaccccacacagctaaggcttccagggtttaggcaagccctgagggacacccgtcccaattagacagggggcccagcatcccaccccagcattctgccttgcaaggccagagggctccctcaccggggctcagggaagagacagcctgcagctccagcaggggaggcctgcagaggcagccacaggtggccaccatgggttgcttggtgccaacttagcgtgagtgggcagagcccgaacacgagggtgccattaccgtcccccatcgtgtacttcttttcctcatagcttgagtctgtgtattccaggagcaggcggatggcgtgggccagctgggagagatggcccgcaggtcgcgtcagagatggagggtccctgactttgtctgcaccagggaagcccccagtttacactgcacgtacccgttgcctcctctacccgcccacacccccaacacacacacagcggcatgcacaggcccttctgaggcgggcagccctgaagaggaacggtccctagagtccgtcccgcagctgcttcgcacttccctcccctgcgccctcgtcccaccgtccagcggaccctcgctcaccccgcggatgtcccagtaccccagtatcatgggcatggtgctggttggtgcggattccgcagacaggcctaaaccgagcagggctcagagtataagagctccggagaggggacaggcggggccttgctgcgaccccgcccctcggcccgccacaacccgaaaggcgccattctggaggcccgggagagggcgcctggggctgcactcagtaagacttcctcccgcctcccaggcccgagggttccgggagcgaagtcagggcgcccagaagcccaaggcggggctcggcctcgccagcttccctacccagccctgaggctccacgagtccatccagcccttgctaggtccacagcgacttggctgtgcgcttgagacaccagccagatcctaacggagcaaagctcttctcccttctcctccctgcccgcggtgtccctcagccttctctccgctgccgagttcccaagggctctgggagactccggctgcaggggtcagactaaaaagtggtggccccaacctgggaatttaattcagcccctgtcactgtaagagcaggacttcctctgatccgaaagctactcccagggcttagtctcccctctagccccgcctacacaggaacagtgtcagtggtataggaaggacccccaggaaaagggccagagtaaaggaaatgtggtctgtgttttctgttaggggccctcgggtactgagtccttcggtcatctggctaagtactgtgtaaattagccacttcgtattttggcacaatttatgaatcgaaatccaaggatcagatccagcaagttggtgaggtatctgaggtgccccctagaaatgtccagccagtccactaggtgaacctcacagagaccggaacagcaacagcaacagtgagtgcagtggccacagagagcaggcgagggaggatggaagaacttcccggaccaaactgagggtccggctgctatttctctaggcccaataacgagatgcagatcaaatggggaggaagagaggttttatttctgcaaccggtaacagggagaaggcctagaaattaccaccagaccaactcaatattacaaagttttccagagcatatataccttctaaggtatatgtctacaagtaagtgtgcattcatctaaagacataagtgattaacttctttaatctgtaactgaggtctgggtcctgaggaccttattctggagcctcagtctatttatttaatctaaatgggtccaggtgcgggggatgattacccttatcttgtctcctgctaaatcacggaggtttggggagttccttcagacgcccaataaacttgtttgtggaggcctggggagtttcttcagactcacaataaaacttgtttaattctaaataggtcctgttaagaattccttcattgttttgtcatgctttaaggcccaggaaaggcctaagcaaaactcttggtgggcttttgttacatccagcctctgtataaaggtactggcttttttcagcttttaatatttaacttcaccactcagtcagtactgaaacagttgttatggaggcctgtgttagtgacgcctggcctgccacagaacctgtccctcagagaagccagaaccctgtcctgctgaggagggaggtcagaggatggtggccagagaggagcctgagggctccccacaggaccacaggcagggaccctcccctctccagatccaccattccggacctggacctggctcctgacccatgtgtgttcttcagtatgagacggtggctccagtggcctttgaagtcacaccgtgatatgtgacccatggtacaacctccacgagaacaatgtccaacctgccaactttcttctttcaaggtagaaggaagactttcaaaagagttgtgcaatggattagcctggggttgactgctttaaaggatattgcaaataataatggacatatggaaatagatgatagacctttaatgagaaatcattttgcaatgtaaaccaggctgttgtgctgcaaaaaaagtagtttttttgttttgttttgttttgttttgttttgttttgttttttgtaaattagctaaaacattgttaggactccagaggatgaacccagtatatcaaaaaagtttcaaaccacctggataatgtgtggtttggggttgagtaaacaggtagacttcatcacatccctattaggccacagaaatcaggtggtcagcaggtggctgctaagttggggcacttggacactgtgtcactgaggctggcaaggcacagtccttgttgcaaacactccccgcccatccagacctgggcagtactcccagctcagatacctggcaggtatccatgtttaggaagcagttttggcctcacagtgaactcttacatagattttcctgcattgaggcaatagcaccagcacagacacaagtgcctggccctaggaaacattatatttctcattatgcattaagagtttatgattctgagttaatccgtcttgagctgtgaaagataagatgtctagatccaattgaaaaaaaaaacgattttttaagaaagctaacactcatgagaataataattttgaaacacatagaaccagtactgttacattagtattgtttgaaatacttgttccttggtgccataaagaaatagcacttgaacataaatttaatttcctcagcaaggctattggggaacctgcccccaatatttcaacgtaggttctttctattttccataagtgtcagccagctgagaaataaagagaaagagtacaaagagaagaattttacagctgggccgccaggggtgacatcacatatcggtaggaccatgatgcctgcccgagcctcaaaaccagcaagtttttattaaggattgcaaaaggggtggtggtgtacgaacagagggtaggtacgaagatcacatgctttaaagggcaaaaagcagaacaaagatcacatgcttctgaggaaacaggacaaaggcaaaagtagaactcctgataagggtctatgttcagtggtgcacatattgtcttgataaacatcttaaacaacagaacacagggttcaagagcagagaaccagtctgaccacaaatttgccagggccaggttttttccccacctgagggtactgcaggagaccggggtgtatttcagtccttatctcaactgcataggacagtcattcattcccaaagcggctgtttatagacctccccccaggaatgcattcctttcccagagtattaatattaatattccctgctaggaaaagaatttagcaatatcttccctacttgcatgtccatttataggctctctgcaagaagaaaaacatggctctattttgcccgaccctgcggacagtcagaccttatggttgtcttctcttgttccctaaaaatcgctgttattctgttctttttcaaggtgcactgatttcatattgttcaaacacacatgttttacaatcaatttgtacagttaacacaattatcacagtggtcctgaggtgacctatatcctcagcttaagaagataacaggattaagagattaaagtaaggcaggcataagaaattataaaagtattaatttgggaactgataaatgtccatattaaaatgaaatatttgcaatttatgttcctctgctgcagctccagccggtccctccgttcggggtccctgacttcctgcaacaaaggccatttttactttctgcagaaagggtacactcgccagcagttttaccacgagagtacactgaacaaaggagaaagggtcatttataacttgatgtgtccaccctactgctgtgtccagtttccactggctggaacgggacctcacattctgtatttgtcctgattggctagcaacttagaactttttaaaagaggcagaggcagaggagaacaaaggaagggggaactcacttgtggaatgctgagtaaggtaaaaacaccttcaaataaggaagaggaacaggctatgacctcatgcttgtttggatcagtataagcatgccagggcaagtatttaggctaaattgtgggagttaagaacataaagtacattaatttctttattacagctagcagatatttcagaatatcagcaccggtctttgaataaattttgcttctaagagaagttacgacttattcttaattagatggggagaaaagtctctttgaagaggaacctctactttactttttacagtattaatatatttggtcattcctttattgaaacaacaattgtcgagatcctactacattccaggcactatgaataaaatgatgcttcaaggacgctgtatttgcccatctccaactacatatcaggtgggaacttcaggggagagattttgaaaatcaggacacatcttcgtgttttggcacactgagaggcagtggagtgggagaggggttgagtgcaggagagagaagtagtgcctgctgttgcaaggactctgaggagccaggaagaaggccaggccccaggtggcttcttcagaagcaggagtagagctctgcggaaggcgggaaggactggactagcggctctccaaagtagctgccatagtggccacaaggatcacaccagcggggaaagtgggtctgtggacggggatcaggagcagcagggatgagggaagctgcaagtgttcagaaggaaggggttgtagccagttgtcaccacacagaaactcagtcccgacagggcctggacttctgcatttgtatgtgctgtctgaactgcaaaatgtggaaaagttctaatttttaaaacaatgctgtgccagacagtcaaaatgcactgggtcccactttaccaagggcctgtaatgggacctctggactggaattcataattccttctgtctcttctgacaacattcttacattctcaaggcaaattaacagaaaatctacctaacatgttgtcagaattttaaaaactaatctattagaaacaaatactgacagacaaaataaaatgtatcctaattcatggaaatgtattaaagtctcttaaatattgtttgggtaaacttaggaaatctgaaatataaagaatcttttcttcaatctgaaaagaagaaaatagaaatcaccatttgaagatgctaccaaatttgaattgagaaaacatttttccaacatgcctgtgataccaagaataaggcaaacaaactagaatttcaagaaagtagaacaggcacaaagttagagatggaaaaagaagtggaagcaataagtgaccttaagagaaatattgaaactttaaccaatttgcttgttatatgtaatagtttattctgttaaaagttaggtaactacacgcctgtaatccccacactttgggaggccaaggcgggcggatcacgaggtcaggagatcaagaccatcctggctaacatggtgaaaccctgtctccactgacaatacaaaaaattagctgggcgtggtggcagacacctgtagtcccagctacttgggaggctgaggccggagaatggcatgaaccagggaggtggagcttgcagtgagccgagatagtgccactggactccagcctgggcgaaagagcgagactccgtctcaaaaacaaaaacaaaacaaaacaaaaaaaacttaggtaattaacacagcattagtacaacaaaaacaaaaaatataattataagttaatacaaatcagaactaagaaagaagatgtagtacaagtgattttttaaattggggaaacatttttctaaactctatgttaattgatgttaccaccaagtggaagaaacacaaacaggccaaaacatgttaagagataaagattctgaatgaagtacaaaaagcgtgataaagaaaaataacaacacaaaagggcattttatcctcagcattaggctggatatgttttcaaaaattttgaaaatggcatggatgacacagattgtcataaactacagacatgacctaacatgggaaggacaacacaggcaaggatgattataaatcactgtcacttaccagcctgaatccaaacatcaggaatgctacctcagagaaaagaaaatagtgtttttcttataatggtgttgtgctcattcggagcttgctgggggagacagagacagaaaggcgggggcaggagaaggaggggcagagggtactctggggggcagaaagcccatgtggaaacattagtatctagggtaatcagagtcgtttctaatctttctacaatttctcaaactctgacgctagatcaatataaaaaggtaaacaagaaaattacatatacatatactcatattattaggtattaatgatacttatattattaacttaaataaatgttccatcaaaaaggaattaactacatgagctacattatgaatctttttattattattaatatttttaatgctagtatccatccagcacaccactggatgtgattcaagtgaaggtgattttgcccctcaggggacatgtaggtctgtctggagactttgttaattgtaaacaccaggaagcgcccgtggtattagtgggtagaggatgggtaggatgctgagcaccctgcgatgctcaggacagcgccccacaacaaagaattctgccctccaaatctctgtacggccgaccttaagaaaccctagtctgtgatccaggacagggtcgaaagctgcagtgggagaaacaaggtcctcgaaattctggctggcaggaagcaggccagaggacccacttccctggacaccagctggcagaagggaggacctagagacaatctcagctggtgcagcccatgcaggcatcaagctgcacatcaggaatcagagaaggagaattgggcaggcaaacggtctactaggtggacaggctgggcagcctggtcttccagggtacagggtagggagtggtgggactctctctgccagtcagacagcaggcctcccctgcagctctcagcacattgctccagagccaggcagcctgagcccagcaccatcgggtaattcttctagagctgctggggcaaagctaggcaggatgaactgaacttattttcaaaaggtatccaccctgactgagaagggtgtgtagaaagttgctacgagtcactgccaacttgtaatggcttgactagaccctccctgcatttaacacccccagaggggtggccaaaagtcaaatatataataagcacctgatgactgccacagacatggatatgagtgatgctagtcaccagacgcagcccccttcccactgctcacccacccttccaagcctgccagagaccctcacccaaaatgaattagcaaaatgtgcaagcagatgttttcatctctggagactttaaaacaatgatcatcaacacggagatttaccaggaaagataatatcttccctcaaaatctccttggagtttactgtgaggcatctcagacagtgctttccatatagaaagaaacccaggtaatagatatgctgcagcgttttatcttgtgtttgattttataaaacttacttattgatgatggtgacccacttgtgtatttttttttttttttttgctatgtgtactagtcaaggttcttcaaaatttgaagtgacaccactaagataagcctgtaggaaatttgtgtgtttatttacaaatttatttatctcttatgggaattggctcacacaattttggagtgaagtcccacaatcagctggctgcaggacagagaagcaggaaagccagcggtatcattcagtgtgagtctaaaggcctcagaacaaaggtgggggtgggaggcgatggtgttagtccctgatcaagtctgaaggccagagaaccaggagtgtggaggtcccaggataggcggagatggatgtcccagtcagagatatcaaatgcacccttcctctgtctttttctcctctctgggccctgacagtttggtggatgcctccttcatttgctgagagaaatcttggctcagtcttctgattcaaaggcgaatcacttctggaaacaccctcacaaacacaataattttaccagccatctgagcatcccttagcccagtcaagttgacacagaaaactaactaaccatcacattatgccacaggaaaccaagaacaaaatcacaattaattacattgtgcctatgggagaaaaagccttgactgccacaagatcattctttaagctttctccaggagttgactaagccaccagtagagaatgggtagagagttttatacaggagattcacaaacaagtagattcatagactgtgattcattggcagaaacatgcacaaccaataaacaaaaaaggtgaaggtagaaaacttgaaggtattctttttttctttttttgagatggaatctcactctgtcacccaagctggagtgcagtgtgcaatggcacgatcttggctcactgcaacctctgcctcccaggttgaagtgattcttgtgcctcagcctcctgaatagctgggattacaggtgcccaccaccatgcccggctaatttttgtatttttagtagagacagggtttcaccatattggctaggctggtctcaaactcctgacctcaagtgatccacccgcctcggcctcccaaagtgctgggaatacaggcatgagccaccatgcccagccagaaaatgtgaaggtgttcttattaaaaaaaaaaaatcataagatgaaatcgcgatctcctaagtgtctaaaatagtgcagaatgatccgtgtaccttgctggtggcaccataaattaagttattgtctctggagagcaatgaaattatctgttgtaataccattaaaccagtctcatcctttaccttgtagtaagcgctttggggatctactcaaagtcaccaaaaacgggaaagaattagtcaaaacatgatcaccctgctctaaaaacaaataaataagttagaaaaatctacacatgccatagcataagaatgactaggcaaaatatagtttatccacaggaactaatatgagatagccatcaatattagaagtaggcatgcatatagactgccaacatatacagtttgttttttttttttttttgagagggagtcttgttctgttgcccaggctggagaggagtggcatgatcttggctcactgcagcctctgcctcctgagttcaagcaattgtcctgcctcagcctcttgagtagctgggactacaggcaagctccaccacgcccaaaaatacaaaaaaaaataattttgatatttttagtagagatgtggtctcaccgtgtacaagccaggatggtctcgatctcctgacctcgtgatctgccccctcggcctcccaaagtgccgggatcacactcatgagccactgcgcccagcccatatacagatttttaagtaaaatgttaggccaggagtggtggctcacacctgtaatcccagcactttgaggggtagaggtgggtggataacttgaggcccggagtttgagaccagcctggccatcatggcaaaacctcatctctactaaaaatacaaaaattagccaggtgtatgcctgtagtcccagctactcaggaggctgaggcaagagaatcgcttgaacccaggaggcagaggttgcagtgagctgagattgcaccactgtgctccagtctgggcaacagagtgagcctctgtctcaaaaaaaagaagtaaaatgttaaagagaaaaaactgtagaatctaagagtaaacacgcacaccagtctcacctatctgaagaagtaggcatgaataaaaatctaaagagaatttgaaatgacaccattaagatgaacgtataatttgcttaataattgttcagtgagcacctactatgctcagggactgtcctaggcgatatgcaaacacctgttacatcagtgaacaagaagagagaaaaacacatgcccttatgatgttgatatcttatgagggaaagcaaacacaagcacacaaataatgtgcatgcttttaactgaagtgtaatacacatgcagtgcacaggccttaatgcatgcttttcattgaagtgtaagatacatacagtggacaggccttaagtgtgctactctgtgggcttttccacttgtgtactcttctggaatcacctcccagataaagagagaaaacttacagcactccaccaggccccttcaataggcaaccttccagttctcacaaagttcaccaatcatctgacctctgataccatggaatcatttccctctcctgattttcgtgtaagtggaatgagattgtgtgtcctttctcctgtctggatccttttactcaatacgtctgtgaggcccatccatgtttttgcacatagcgttagcatgtttattttcattataccagagtaagtattccattatatgaatgatggaattatgttgataaaccacagttcatttgttcatggaatttggattgttttcagttattggctatttaaaataaagctgcttttggcatcactacacctggttcttggaatccattaggactcattcctgtagggtgggatttctgggtcctgggtagatgtaggtttagctctagttgacagtgccagtattccaaagtggctttcccaccagcaatgcatgacagttccagttactccataaatcaccaacaattgtcggtcctttcaatgtcagccattctagtggggatacagtaggatcttaggggtttttaactttctgctgtctaatgatgttgagcaccttctcatgagcttagtggccatgtggatattcacttttgggaaatgcttgttgaaattctgactcattttttaatgagttgtattttcggatatatagtttttggagaagagacctttgttgatttgtaacaagatctccctgctgagccttgcgtgaatacatgacccacagactcaacagctatttctggtatcacagcttaggaactgctaagctatgagaatcttcctctgaagcagttcactctccagtgagattttaagttacttcaacaagaggcaatgcagattgctgtcagggtggaggaagcctatgcttagcatgttcctgcaaggatgtgaatcaagaagagatcaacctgtagatccccacacagcctttggaattatagacctcagaatgacaaacagaaaaagcgtaaaaaagcaaaagatatagccaggcagagtggctcacgcatgtaattccagaactttgggaagccgaggtgggtggatcacgaggtcaggagttcgagaccagcatggccaacatggtgaaaacccgtctctaaaacaaaaatacaaaaatcggctgggcacagtgtgcacgcctgtagtcccagctactcgggaggctgaggaaggagaattgtttgaacccaggaaggggaggttgcagtgagacgagatcacaccaccacactccagcctaggcgacagagtaatacttcctctcaaaataacaacaataataataatttttaaaaagcaaaggatgcagtcagactggcatcgacctactcaccagaaatgctcaattctagaaaaaagtgaataaatctattagaagtctgaaggttttcatcatagaattctatccccagctgatatggtttggctgtgtccccacccacatcttaccttgaattgtaataatccccaggtgtcaagggtgggggccgggtgcagaaaactccatcatgggggcagtttcccccatactgttctcacggtagtgaataagtctcacaagatctgatggttttataagtgggagttcccctgcacacgccctcttgcctgctgccatgtaagacattactttggtcctcattcgccttctgccatggttatgaggcctcccaggccctgtggaactgtgagtcctttaaacctctttcctttatcaattacccagtctcgggtatgtctctattagcagcgtgagaacagactaaatcctcagccagagtattcagtaagcatgtaaaggcactttcagttatgcaaggactaaagacatttctctcacaggtacccaggaaagagaggaagtaaacccagagagaacgtcctgatgacagctgtgcagcaagcctggagagggtacctggaagccatctgagaaaaacaatagagattgggtcctggagacctaacagaagtgatgcagtcatatagtggagaaggtaaaagggaccctggttctctctgatggagattccacccccttggggtcctattcaatgagacacaagctctgtgcaattatggaaagtactcaatacggctggatgaatgaatgggagatccaggccaagaaagagacatcctttccacagtgcctcatggggcttcctgctctgagtctctcaggaatgggagcaagaattctcaagcgccttactatgcccagcagaatcctttagaacccagccactgccaggatcaccagccagtcccttggaagaggcagtgacagacagacagacagtctgggtagatggccaggagcaaataagacaagacacacagcaaatgtggttgaggctttacttccatcaggtaggcctaaggcagggaccatgctgcaggagcagggcctgtaaaccagtcaatgctgctccttcatgcaacacggggacagctcggggctgagctccacaggcgaggggccaggcaggccctttaaagcagacacaaccactaacaggaaggaaggtcaggctggctttagtgcagggaagggtaatgatgggaggggatgttcacgaaggatagtgggtagctgaggcttcaaagggcctgcatgggacaggggtttagggggagtgaagagggacaatgaagtcttgggagtaaagatgggaataaacaggagaaaggaacgaggaggcaggtgctgggatgcagagtccagctgcgctgcacagcctgggcagcaggctgagtatgggctcctcactcccacctcctggccttcaaggccctacttgttgccccagacagccatctttgtgaacacaggtcttgggaggaagcggctggacttcatgtaggcagagatcttctccaagccctgagaggggggaaaataaggccagaactcaaggtcagctgcccatgagggctggcacaaccccactgcagacacagcaggtcagggccctggccacagcctttccatgtgggctcaagtgctgaggcatcacacagcttcttagggtccccagatctgcggagttaactcttccaacttcctttaagtaatgttgtaccacagtaacagctaacacttatgaagattctacacggtgctgggtgctctgtacccaagagaaattacaacgtaagcttagaaaacccactcgactactctgcaaggctgaatctattatcaccatttgtcatgtgggaaacagaagttcagcaacttccttacatttaaatacctagtaaggggaatagaacttcaatgtagcctggttctgtctctctccagttctggcctctttctgccaaattctcttcttcttctgccccaccacccctcacaccaggccctgtcactccttgactgcacaaacagtctccttttccacagggaggctcttggcttgtctgggtatagagacacagaagggacctccagcagaacaaaggggctctgaacacactcacccaatgtctcagtaaaacctggatgaaagtggaatggggagtggcagtgcacaagaaaatcaaaggcgagactaggctctaatcattccccttaaagcagaaagtccaaggttctccatctggtatcttagcaaaatctcactgtgccctaacccatggcatctccagccctgctgcagatcaggctacatgacactcagatgtgctgtgcacaatcttaccaggattgaatgcccagataaggtattcaaaaacatccggctcacacctgtaatcccagcactttggaaggccgagacgagtggatcacctgaggtcaagagttggagaccagcctggccaacatggtgaaaacccatctctactaaaaatacaaaaattagctggtcatggtggcgggcacctgtaatcccagctacttggtaggctgaggcagagaattgcttgaacccaggaagcggaggttgcagtgagccaagatcgcaccaatgcactccagcctgggtgacaggcttgaccctggcacataacgggccatagctctcagctctcctccatccctcccttcccaccacctcttacctaactccaaggccactcagcctggaccagaaccgcagaaccagagctgatgggaactagctgagcaagcagatctcttgatggggagtggaaaagggaaacagattcaaggctcagaagcctgttggacaaatgcctcaaatccccaaatctgtagtaatgtaggatttgctgctctcaaacaagttcagcacacacttcacaggctgctctgcctgcaggcctctcactgcactcatttccaggatggggctgtcatctatgcatctcccactgtgcctcacaccctgcctcatgcagaggagaaggtcagtgaacaggagtaaaatcagcgtgctactcatctcacagggggaacacctggaacattggagggaaaaaaagagaggaagaggccaggcgcggtggctcacgcttgtaatcccagcacttagggaggctgaggtgggtggatcacctgaggtcaggagtttgagaccagcctggccaacatagcaaaaccccatctctactaaaaatacaaaaattagctggacgtggtggtacacacctgtaatcccagctactcaagaggctgaggcaggagaatcacttgaacccaggagacagaggttgcagtgagccgagattgctccactgcactccggcctgggtgacagagcaagactccgtctcaaaaaaaaaaaaaaaaaaagaggtagagaaggaaagtgctccagcacagggcatttcagcataagctccttcaacaggggcacccacatatgtaccttaatccagctccagccacagccctggctctcaggattccccacatgggggttcaatcatacaaagtttctgggtgtagctgtgaatactgcctgtgggttcagtattgaacatcaatgctactgtgtgtcagtcctactattgccagctgcatccgtggtcctgaagtgctctgcctcttccaaagtgtcctgggctatgaaacccaggctggacaaggcctgaggcttttcttttgtcacaaaacggagagccctgactgccaaccatagatccagaggctcgcagggagcccagcacagcctccactggctctgggaccctgagcaaaggactgaccctctcattctcagtttctttgtctctaaaatgggggagataattctaccaccctgtaggaatgttgtaaatttcaaattcaacacaataggtataatgtgctgggtacagttcgtggcatgaagccagcactcaataaatgcaagttattcttttcttcttttttttgtttgagatggagtttcactcttgttgcccaggctggggtgcaatggcacgatcttggctcaccgcaacctccgcctcccaggttcaagagattctcctgcctcagcctccctagtagctgggattataggcatgtgccaccacgcccagctaattttgtatttttagtagagacaggggtctcaccatgttggtcaggctggtctcgaactcccgacctcaggtgatccacccaactcggcctcccaaaatgctgggattacaagtgtgagccaccatgcccggctccttttttttttttttttttttttcttgagatggagtcacactctgtcatacaggctggaatgcagtggcacgatcttggctcactgcaacttccacctcccaggttcaagtgattctcctgcctcagcctcccaagtagctgggattacaggtgctcgccaccacgcccagctaatttttgtatttttttttagtaaagatggggttttgccatgttgggcaggcaggtatcaaactcctaaccacaggtgatctgcctccctcagcctccaaagcgctgagattacaggcgtgagccaccgcacatggccaaaaatgtgagttattctttgtgtagctccaggactgggaaagcatcacaaaggagggaaaggaataagagacataaagagaaaggaggctgggggcatcacctcaaatcgggagatgaagtccttcaggtttgggaaggcatccaggcagctgggctcaaatacttggtttctctcaaggacatcataagcgatgaaatccacaaaggtgatctggataaggccaaggatatgtgggctgaaacctccataatataggaagtatgacaaatctgtggtgtccccatcacacgcccccattaccttgtccccaagaaaccatggctgcttccccagaaactgtgagtagagcttcagcatttcagggagtgcctgcaggtattctggtttcagtttctcctgaggcagaaaacagctgtcaacaccctcagacacaagctccctgcacagatgcggcctcccagttttgtgcatggccccagctggccgcgggcaagcagtcatcttccccacagctggaactgggtcactgtccaggcttcagctggatacatcagcatttattagactccaactgggtagctggagtctttctgaggtgatgggatggcagagcggaatgtgacactgtccctaaatctgtccccactgacctcccaacaacctctgggtcagccccaggagcacctgagcacctggtaggctctggaccagatatgagcagaatcaaggatgcaaagaactaaaggagctcagagaaagaagtagaaatttcctcctagttgggcagcagtagaatacaatggacattgagtggtttctggaaagatgaggagatattcagaggataagtggaagaaaggagggggaaaaagagcctctggctgcccctaagctgtctgttaggaaaattcgctgatctcagtgtatgtcaagagagcactcggaagacaccaaaatttcaggtgccattactcctttgttttgcacttgttttcctcatagtttgtgtattcctaaagcaggcagatgatataaaccactgggagagatggcccaaagcacaggtcatgggtcagagatggaagggattctggaagtaaccattactgtctagagagtgaattcataccctggggagtcaggattctataagtagattggtggggaattgatttagaggttttatttgacacgactttggggtttgagggtcactctggtggcagtataggaaatagcttgaaggatgagtagcactaaagagttcaaacagaaaacaggacccgtgtatttggcaggaccacagtcttggctcctgatcaaaatgagaggtgaagctggctgggcttctgggtcaggtgaggacttggagaacttttctgcctagccaaaggattgtaaatgcacaaatcagcgctctgtgtctagctaaaggtttgtaaacgcaccaatcagcactctgtaaaaacagaccaatcagcactctgtaaaacggaccaatcagcactctgtaaaatggaccaatcagctctctgtaaaatggaccaatctgcatgtgggtggggccaaataagggatttccccccccacctccgcccttcccaagcagcagtagcaacctgctcaggttgtcttccaggctgtggaagtgtattttctttcactcgtcaccataaatcctgctgttgctcactctttgggtttgaactacctttatgagctataacattcactatgaaggtctgcagcttcactcctgaagccagccagaccatgaacccaccaagaggaataaacaactccagacgcgccacctttaagagctgtagcactcactgcgaaggtctgcggcttcacccctgaagtcagcaagaccacaaacccaccggaaggaagaaactgcggacacatctgaacatctgaaggaacaaactccggacacaccatctttaagaactgtaacactcaccgtgtaggaccgcggcttcattcttagagtcagcaagaccaagaactcaccagaaggaatcaattttggacacaaactctgcagggaaaggggagactgggagtagggaaggggagactgggagtggggtggggggacttacaaaatctgggtcatagcagagtttggccagctgcatacggctgtccataaactggttctccaaaatgtcttcgcgaatctgctccttttctgattccccgcctaccctcgtaaagcagattcactctcagcctcacagcccagccaagccgaggaggaggccacggtccctccccgcatcttgcaaccaaatctactcacacaggttgtgcttgcgggcaatgtaccgcaggatggcgttgctctgggtgatcttgtgagtcccatcaatcaagtagggcagctggatagatgggaaaccggggaagctcagttgggccaccaggctccccagcatccccttcccatatgcaagggcaaagaggagaccagggcctgctcatggcaggcctgagatagaaatactgtcacactaataaatgagctgggacacagaacatcatgcaagggctgtgtagacagccaagagtgagaggaattgggcagaagactcctgatcttaacacctctaagctggggagaggagatgcagtcagagacacgtcgcactttcccccaaaaccgccccttcccctgcacctacattgggaaagtccaggcccagcttgaatttttcattcagccactggcttctgtcataatcaggagctgtggtggggaagatgaagtgaaaacaaacctccccaaagcccagccctccttcctggggaccctcccaaggagccagtggcaagacccctgagacaggtggatctagaatctgaccactcagtcccacatcccaggattcagagaaaaagaattccagtgagggctctggaccccacacagctaaggcttccagggtttaggcaagccctgagggacacccatcataattacccagacgggggcccagcatcccgccccagcattctgccttgcaaggagctccctcaccagggctcagggaagggacagcctgcagttccagcaagggaggcctgcagagtcagccacaggtggccactatcggttgcttggtgccaacttagtgtgagggggcagggcccagactcgagggtgccattaccgtcccccatcgtgtacttcttttcctcgtagcttgagtctgtgtattccaggagcaggcggatggaatgggccagctgggagagatggcccacagctcgggtcagagatggagggtccctgactttgtgacgactctgcacaaggggagccccatctcctcctctcgttcctgcctcacccgcccccaccccgcacgcccagccacacgcacagacagcggcaagcacagaccccgctgtcagggacagccctgaagaggaaccgtccctagagcccgtcctgcagctgctccacacttccccgcccccacgcacccccgtcccaccgcccagcggaccctggctcaccccgcggatgttccagtaccccagtgtcatgggcatggtgctggttgctgtggattctgcagacaggcctcagcggggcggggctcagcgtttgtgagaggcccagagagggtagaggggaagccttgctgcgaccccgccccacggcccgccctgcccccgaaacgggccaatctggaggcctggagcgcgctcatggggctaggagtaggatctcctcccacctcccagccccgtgggtttcaggagagagatcaggacgcccagaagcccagggcgggggagaactggttgagtccaggggttcaagactgaactgagctatgatcgcgccgctgcactctaggttaggcaagaaagaaaggctctctctaaaacagagagattctgaataaagtaataatagcctaataaagaaaaataacacaaaagaacatttggtgctcagggattcactggataagttttcaaaacttttcaatgtatgatagagattgttataaactgcggacatacgtggcatgacagacctaacgtgggaaggacaacacaggcaaggatgattataactcactgtcacttatcagcctaaatccaaacgtcaggaataccgcctcagagaaaagaaaatgatgtttttgtcataagtggtgctgtgctcctagggagcttgctgggtgggaagagagacagaaaggtggggagcaggggctggtggacttggggagggaggagaaagcccatgtggaaacgttagaatctggggtaatcagaggtctttgtattcattcgttttgtaaatttctcaaactctcatgttaaatcaaaataaaaagttaaaaaaaaaaaactaccaggacagacatacacaaatattattaactgaaataaatgttccatcaaaaaggacttaccttaactacatgagttatattatgatttctattattattattattattattttaatattagtatccatccagcacaccactggtcttcaagtggaggtaactttgcccctcaggggacatgtaggtctgtctggagactttgttaattgtcaccactgggaagcgcccatggcatctagtgggtagagtttgcgtaggatgctgaacaccctgcaatgctcaggacagtgccccacaacaaatgattttgccctccaaaatgacagtactgctgaggtgaagaaaccctagtctaaaacccagccaacgtggtgaaatcccgactgtactaaaaatgcaaaaaaattagccaggcctggtggcgggtgcctgtaatcccagctactcaggaagctgaggcaggagaatcgcttgaaccagggaggtggaggttgtagtgagctgaggttgcaccactgcactccagcctgagcgacagagtaagactccgtctcaaaaaaaagaaaaagaaacccaggaatgggtcaaaagttgcagtgggagaaacaaggtcctcaaaattctggtggcaggaagcaggccaaaggacccacttccctggacaccagctggcagaagggaggacctagagacaatctcagctggcacagtccctgcaggcatcaagctgcacatcaggaatgagagaaggagaattgggcagggaaatggtctaccaggtggacaggctgggcagccttgatctttcagggtacagggtagggagtatgggactctctctgccagtcagacagcaggcctaccctgcagctctgagcacattattccagagacaggcagcctgagcccagcaccagagtgggtaattcctctagagctgctggggcaaggctaggcaggatgaactgaacttatttttgaaaagtatccacccagcctgggaagcatggtgaaacattgtctaccaaatatacgaaaattagctggccatggtggccctttacctgtggtcccagctacttgagaggctgaggtgggaggatcattggagcctgagaagtcgaggctgcaatgagccgtgatcacaccactgcactccagcctgggtgacagaatgagaccctgtctcaaaaaaaagtaataacaagtcaaatttaaaaagaaggtatctacctgactatcgtgtgtagaaaggtcccaaaagtcaatgccaatttgcaatgacttgactggactcttcctgcatttaacacccccattaggggtggccaaaagtcgaatatataataagcacctgatgactgccacagacatggatatgagtgatgctagtcaccagaggcagcccccttccactgttcacccacccttccaagcctgccagagaccctcacccaaaatgaatcgacaaaatgtgcaagcagatgttttcatctttggagtctttaaaataatgatcatcaacagggagatttaccaggagaaaagagaatatcttccctcaaaatctccttggagtttactgcaaggcatctcagaaagtgctttccatatagaaaagcccccaggtaatagatatgctgcagcgttttatcttgtgtttgattttttaaaacttattgatgatgagtttttgtgtgtgtgtgtgtttttgtttgttgttgttgtcgttgttgttttttgctatgtgtactaggtcagggttcttcaaaatttgaagtgacaccactaagataaggctgtaggaaatttgtgtgtttatttataaatttatctatctgttatgggaattggctcacacaattttggagtgaagaagtcccatgatctgccgactgcaggatagagaagcaggaaagccagtggtatcattcagaggctaaaggcctcagaacaaaggtgggggtgggaggcgatggtgttagtccctgatcaagtctgaaggccagagaaccaggagtgtggaggtcccaggataggcagagatggatgtcccagtcagagaggtcaaattcacccttcctgtctttttcttctctctgggccctgacagtttggtggatgcctccttcattgctgaaggaaatctttgctgtcttctgattcaaaggcaaatctcttctggaaacaccctcacaagcacaatgattttacctgccatctgagcatcccttagcccagtcaagttgatgcagaaaattgaccatcacattatgccacaggaaacctagaacagaatcataattaattacattgtgcctattagagagaaggtcttgactgccacaagatcattctttaacctttctccaggagttgactaagccacaggtagagaatgggtaaacagagagttttatacaggagattcaaggacgggtagattcatagactgtgattcattggcagaaacatgcacacactataaacaaaaaaggtgaaggtagaaaacttgaaggtattctataaacctataaaaatattataagatgaaactgcaatccctcaagtacctaaggtagtgcagaaagatccatgtgacttgcaggtgtcaccatgaattaagttattctctctgggctgggcgcgatggctcaagcctgtaatcctagcactttgggaggctgaggcaggcaaatcacgtgaggtcaggagttcgagaccagcctggccaatatggtgaaaccccatctctactaaaaatacaaaaattagccgagggtggtggcacacgcccgtaattccagctactcaggaggcagaggcaagaaaattgctggaactcaggaggcggaggttgcagtgagctgagatcgcgccactgcactccagcctgggtgacagagtaagactccacatcaaaaaaaaaaaaaaaaaaaaaaaaaaaaaaaaaaaagctattctctctagagagcaatttatctatgataataccataacaccattctcaccttttgccttgtagtaattgcctttggggaccaactcaaataaagtacccaaaaaccagagaaggaattagtttaaacatgatcactctgttctaaaaataaataaatgagaaaaatccacatgcccaatagggtaagaatgactaagcaaactataatttatccacaggaagtaatgatagccattaaatgctagaaataggcatgcatatagactgccaacatgtacagattcttaggtaaaatgttaaagagaaaaaactgcagaacctaagagtgtatgtgcccatcaatctcacctatccaaagaagtaggcataaataaaaatctaaagggaatttgaaatgacaccattaagatagagcctataatttgcctaataactattcagtgagcacctgctatgctcagggactgtcctaggtgatatgcaaacacctgttacatcagcgaacaagaagagacaaaaacacatgtcctgaggacgttgacatcttatgagggaaagcaaaccaacacaagcacacaaataatgtaagacatatgagcatgcttttcattgaagtgtaatacacatacagtgcacaggccttaatgcatgctttttttttttctttttgagacggagtcttgctctgtcgcccaggctggagtgtgcagtggcgtgatctcagctcactgcaacctctgcctcctgggttcaagcaattctcctgcctcagcctcctgagtagctgggattacaggcgctgccaccacgcctggctgatttttgtatttttagtagagacggggtttcaccatgttggtcaggctggtctcaaacccccaacctcatgatccacacgcctcggcctcccaaagtgctgggattatagacgtgagccaccgcacctggccaagcatgcttttcattgaagtgtaatacatacagtgcataggccttaagtgtgctactctgtgggcttttccacttggatactcttctggaatcacctcccagataaagagagaaaacttacagcactgcaccaggagccttcaataggcaaccttccagttctcaccaaggtcaccaatcatctgacctctgataccatggactcatttccctctcctgaatttcgtgtaagtggaatgagatcgtgtgtcctttctcctgtctggatccttttactcaacacttctgtgagacccatccatgtttttgcacatagtgttagcatgtttattttcattatagcagagtaaatataccattatatgaatgatggaattatgttgataaaccacagtttatttgttcattcttctattcatggaatttggattgttttcagttattggctatttaaaataaagctgcttttggcatcattatacctggctcttggaatccattaagactcattcctatagggtgggatttctgggtcctgggtagatgtatgtttagctctagttgacactgccagtattccaaagtggctttcccaccagcaatgcatgacagttccagttactgcataaatcaccagcaattgtcagtcctttcaatgtcagccattctagtggggatacagtaggatcttatgggtttttaactttctgctttctaataatgttgagcaccttctcatgagcttgttggcaatttggatattcatttttcggaaatgcctgttcaagttttgactcattttttaatgggccgtatttctggatatatagtttttggagaaaagacctttgttgatttgtaataagttctccctgctgggcatcacctgaattcctaacccacaaaatcaacaactatttgtgataacacagcttgggaactgctgagtgggagaaccttctggtgaagcagctcacagtagagcttgcgtcctagagatctaagctgtgatggagtcagtcagtgtggaagacagaaggatcctggttctctgtgatgggaattccaccacctctgggcatctaagacgtactcactgagacctagctctgcctaaccatggaaagcactgaataatactgaatacataaaaataaatgaagatctaggcccagaaagaggcatcctttctatagagcactttatgtggcttcctgctctaaggcagtcagaaaagagatcaagaagttggaaacagggccaggcatggtggctcacacctgcaatcccagcaatttgggatgccaaggtaagaggatcgcttgagctcaggagttccagaccagcctgggcaacataaagagacacatctctacaaaaatttttttaatgtggccaggtgtggtggcacacctatagtctcagctatttgggaggctgaagtgggaggattcttgagcccaggactggatgttgcagtaagctacgatgccgccactgcccttcaccttgggaagcaggagaccttatttcaaaaaaaaaacaaagttggaaacaccttactaagcccatcataatctctatgaacccagctactgccagggtcaccagccagtccctcagaagaggcagtgacagacacagtctgggcagccaggagcaaataagacaagactatagcaaatgtggctgaggctttattttgatcaggtaggcctaaggcaggggccatgctgcaggagcagggcctgtaaaccagtcaatgctgtcatgcaacacggggacagctcagggctgagctccacaggcgaggggccaggtaggcccttcaaagcagatacaaccactaacaggaaggaaggtcaggctggctttagtgcagggaagggtagtgttgggagggggatgttcatgaaggaaagtgggtagctgaggcttcaaagggcctgcatgggacaggggtttaggggaagtgaagaggcccaataaagtcttgggggtaaagatgggaataaacaggagaaaggaacgaggaggcaggtgctgggatgcagagtccagctgcgctgcacagcctgggcagcaggctgagtatgggctcctcactcccacctcctggccttcaaggcattacttgttgccccagacagccacccttgtgtacagaggttttgggaggaagcggctggacttcatgtaggcagagatcttctccaagccctgagaggggggaaaataaggccagaactcaaggtcagctgcccatgagggctgggacaaccccactgcagacacagcaggtcagggccctggccacagcctttccacgtgggctcaagtgctgaggcatcacacagcctcttagggtccccagatttgcggagttaactcttccaacttcctttaagtaatgttgtaccacagtaacagctaacacttatgaagattctacccggtgctgggtgctctgtacccaagagaaattacaacataagcttagaaaacccactcgactactctgcaaggctgaatctattatcaccatttctcatgtgggaaacagaagttcagtaacttcctcacactcaaatagctactaagggggacagagctgcaacgtagactggttctgtctctctccagttctggcctctttctgtcaaatccccatcctcccccaaatccctcaggaggtcctgtcactctctgactacacaacattctcctgtttcacagggaggcacctggcttgcctaggtttgcacacacagaggggacctctgaaaatgtctgtgcagcctggaagagagcccagatctcacccaaggacctggcaaaacctggatgaaagtggtcaaggtgtgttggaccagcaaaagaaacgccagaccaggctctaatcattccttttaaagcagaaagtctaaacctccccacctggtacctgagctaaacaccactgtgccccaacccacctcatctccaaccccactacctcagaccaggctgtatgacactcaggtgtgctctgcacgatctttccaggatcaaatagccgtgtaatttctaaggattcagagagagccagggctgcctattcccaaagggccaagcccaggtggaccatatgctgcagccctcagctctcttcccctcccactacctcttacataaccccaaggccactcagccgagaccacagccagagctgatgggaactagctgagtaaccagatcccttgatgggtagtgggaaaggaaaacaaattctaggctgaaaaacttgttggactaatgcctcaaattctcacatctgtactaaaagtaggatttgctgctctcaaacaaattcagcacgggtgagggatggttccccatgagtgaggggcaggccccaggcctggtacccaggtggtgccacacacctcacaggctgctctgcctgcaggcctctcactgcactcatttccaggatggggcagtcatctatgcaactcccactgtgcctcacaccctgcctcatgcagaggagaaggtcggtgaatagcagtaaaatcagtgtgctactcatctcacagggggaacacttggaacactggagggaaaaaagagaggaaaaaagataaagaaggagagtgccccagcacagagcacttcaacatacactccttcaacaggggcacccacatatgtaccctaatccagctctggccacagctctggctctcaggattccccacgtgggggtctgatcgtgcaaagcttctgggagtagctatgaatactgcctgtgggtttcgtactcgacgtcaatgccaccgtgtgtcagtcctactattgccagctgcatccatgctcctaaagttctctgcctcttccaaagtgccctgggctgtgaaatccaggctggatgaggcctgaggcttctttcttttgtcacaaaagggagagccctgactgccacccatagatccagatgctcacagggagcccagcacagcctccactggctctgggacccagaacaaaggactgaccctctcattcaaagttttcttatctctaaatgggcaagataattctgtcaccctgtaggaatgttggaaatttcaaattccacacgataggtataatgtgctgggcaaggttcctggcatgaagccagcactcaataaatgcaagttattctttatgtagctccaggactgggaaagcatctgaaaggaggtaacggaacaaggggcatcaaagagaaaggaggatgggggcatcacctcaaagcgggagatgaagtccttcagatttggaaaggcgtccaagcagttgggctcaaatatacggtggaggtcaaggacatcataggcgaggaaatctacaaaggtgatctgcagaaggccaagaatgtgtgggctggaatctctgtaacgtaggatgtatggcaaatctctagtgtcctcatcacatgcccccattaccttgtctccaacaaaccatggcctcttccccaggaactgtgagaagtgctgcatcattgtaggaagttcctccaagtattctggcttcagtttctcctgaggcagaaaacagctgtcaccaccctcagacacaagctccctgcacagatgtggcctccccagggctgtgtatggccccagctggccacgagcaagcagccatcttccccacagctggaactggcttcagctggacataccagcatttattacactccaaatgggtagctggaggcttttgtggtgatgggaaggcagagaggaatgcaacactatccctgaatctgtccccactgacctcccaaccaccctcctctgggtcagcccaggagcacctgagtacctggcaggctctggacccagacatgagcaaaatcaaggatacaaagaactaaaggagctcagagaaagaagttgaaagttcctcctagtgcagtggcagaataatacaatggacattgagtagtttctggagagatgagattttgagaggctgcatggaaggagacagaggaacaaaggccctctggccgcccctgagctgcctgttaggaaaatgcattcatccctgtgcgtgtcaaggagaagcactaggaaaagaatagcacggaattccacagaggagggtgctattacatcccactacatacttctttaccccatagtgtgagtctgcgtattccccgaacaggcagatggcatgaaccagctgggttagatggcccgtggcagaggtcaggggtcagaggtgaagggaattctgaacgcatgactttattgtctaggaactgaattgctagctttcataggacttcctatgattggtggggactgatttcaagtttttattttataagtttaatgatatgacagtcactctgctacagtacagggaaccctcagaggatcaatagcagtgaggagttcaaattgtgaataggaccagagtagatgggaatacaagcctggcttctggtccagaccagggagggactcacaaagtcagggctgtagcagactctggccagctgattggagacgtccatagcctggttctccaaaatgtccacacgaatcttctcctcttctgtctccccacctgccacccacaaaacacagactcactctgagcatagcaccccaatccagccaaggaggatgccaccttccccccacacactgcagccaaccacactcacacaggttgtgcttgcgggcaatgtagcacaggatggcgttgctctgggtgatcttgtgagccccatcaatcaagtagggcagctggatagatgggaaaccggggaagctcagttgggccagcaggctccccagcatccccttcccatatgcaagggcagaggggagaccaggcactcactgtgcccgctcatggcaggcctgacatagaaatactgtcacatgaacaaatgagctgggacacagaacatcatgcaggggctgtgtagacaaccaggagtgagaggaattgggcagaagactcctgatccgaacacctctaatctggggagaggagatgcagtcagagacacgtcgcactttcccccaaaaccgccccttcccctgcacctacattgggaaagtccaggcccagcttgaatttttcattcagccactggcttctgtcatagtcaggagctgccgtggggaagaagaagtgaaaacaaacctccccagagcccagccctccttcccggggaccctcccaaggaaccagtggcaagatccctgagacaggtggatctagaatctgaccactcagtcccacatcccagggttcagggacaaagaatcccgctgagggctctggaccccacacggctaaggcttccagggtttagggaagctctgagggacacccaacccaattaaacagggggcccagcatcccgccccagcattctgccttgcaaggccacaggactccctcaccggggctcagggaagggacagcctgcagctccagcaggggaggcctgcagaggtagccacaggtggccaccatgggttgcttggtgccaactcagcgtgagtgggcagagcccggacacaagggtgtcattaccgtcccccatcgtatacttcttttcctcgtagcttgagtctgtgtattccaggagcaggcggatggcgtgggccagctggaagagatggcccgcaggtcgtgtcagagatggagggtccctgacttggtgactttctctgcaccaggtgagcccctagtctacactgcactgcaccccccccccaccccggcgcacgcacacacacacacacacacacacacacacacacacacacaggcatgcacaggccctcctgtgagagatagccctaaggagggaaccgtccctagagccggtccccagccgctcggcacttcccgcccacgcgcccggtcccacagtgcagcggaccctcactcaccccgcggatgtcccagtaccccagtgtcatggacatgatgctggttggtgtcgattctgcagacaggcctcagctgggctgaactgcgacctcctctggggttcccggcacgcaggggctggacctagcgccagacccgccccctcggccccgctgcgcccgccgatcttcaaggtcgtcacttccaaccggccgatcttcaaggtcgtcacttccaaccaacaggcgcgggaggcacggagcaggttgctggatcctcactggctggaaggagtaagatccaccgccacctccgagtgttcagggagcaaggtccggaagcactaggaggggctcggcctcgccagcttccgtagccccgccccgccccgctccgcttcggacctctgctgggtccccagggactcggctgtgcgcgtgagagtaaagccagatcgtaagagaaaagttcttcccccgtttcttcttctccggacgtcgcccagccttctgcctctcggctgccgagttcccacaggctctgggagactgaggctgccagggtcagactaaagagaggtctcagagagtttaattcaacacttcttggctactaagtcttagaagtctgatggtgtgctctctctgctgagttggggagcgtgaatggaggctatgtcaccgaagctgatagagctcagtctctgttgcagatgctcccgacccttttgcattgggccagttccccagctctgagactgggtccaggctcaggaagtggcctatgtgtcaaggtggattcccatgtaggtttgcctacagcaagacgatagcaccatgcacacacaagtgcttggacttctgaatagttatacttctcattatacattaagacattaagaatgtgtcattctgagtcttttttttttttttttttttggaacggggactcgctgtgttgcccaggctggagtgcagtagggcgatctcggttcattgcaacctccgcctcccgggctcaagcgattctcctgcctcagcctcccgagcagctgggattacagacgagcgccacgacgccctgctaacttttttgtattttctgtagagatgggatttcaccgtgttgaccagactggtcttgaactcctggtctcaagtgatccacccgcctccgcctcccaaagtgctgggattacaggcatgagccacggtgcctggcctgtcattctgagtttatccatcttgagctgggaaagataaaatgtctaggtccaattgtgaagatgattttttaagaaagctaacacttatgagaataattatgaaagaaacagaattgatattgttacattagtattcacccatttggtcattcctttattgaaacaacagttgttgagatcctaccatattccaggcactgggaataaaacaatccttcaaaaacactgtactttggccacctacgactagaagtcaggtgggaactacaggggagagattttgaaaatacgggacacttcttactgtttttgcacactgagaggcagtggagtgggagaggggttgagtgtaggagagagaagcggtgcctgctgttgcaaggactctgaggagccaggaagacggggcccaggtcccaggtggcttcttcagaaggaggagtacggctctgcggaaggcgggaagaactggaccagcaggatctccaaagtaggggccacagtggccacgaggatcacaccagggcgggaagtgggtctgtggacagggatcgggagcagtggggatgagggaggcggaagtggtgccctggggaagaatgtgcacgctgttgtcaggttattttctgcatgcctgtgagatctaaccggttcattgtgtgttcacattctgcatttccttacttaccttccgtctcagcctttccataattgaaagaggggttttagagcctcccattgttaatgtagaactgtctatttctcccttcaattctgtcaaattttgctgcatatatcttgttttattcctactatgggaattgcatcttgtatgagagaaatcaattatccttcctggatggtccaccagattatctgtgctttcctcattttagaagcttctgggtatccttccttccttccttccttccttccttccttccttccttcccttccttccttccttccttccttccttccttccttctttctttctttctttcctttctttctttcttttctttctttcctttctttctttctttctttctttctttctttctttctttctttctttcttttctttctttctttctttctttctttctttctttctttctttcttttctttctttctttctttctatggagtttcaatcttgttgcccaggctggagtgcaatggtgcgatctcggctcaccacaacctccgccttcctggttcaagcaattctcctgcctcggcctcccgagtagctgagattacaggcatgcgccaccatgcccggctaattttgtatttttagtagagacactgtttctccatgttggtcaagctggtctcgaactccgatgtcaggtgatctgcccccctcggcctcccaaagtgctgggattacaggcgtgagccaacgcgcctgggcacttctgtatatctttttcaagaaaacatgatttcctggtgggatgatagttttaaatatctggcaatctctaacaattcattggaatcacagggtagcagtggacagaatgatcctgatgtcatgtaagaaacaggattttagtggagcactgattatactggtattaagtatcatttattacaataggaatgagtcacttttgtgttcctcaagatggacagattccttgagtggaattaaatagtttctgcaaagcaggctcagatgtctctgaagaaagcaaacaaccgtggtagccagaatagacccaatattagtcctgatttattggccttgtaatggcctcagcagaagcaatttgttggcacgaagtctaagcacaattgttgcaagactgaaggttgggatgaatgatgtgagtgaaacataattaccataaggagccagttaaagggaggttgccttgtgaaggttttaaaaggacagctatgaaatttttgtcagcagggttgttagattcagagtggcagcaatggtttgggaaaaaaaagtataagaaaattttgttttctgttaagtggtagatagagaacataagaggtcagaagtaacaggatgagggccaggtacagtggcttgtgcccatcataccaggtacttgggaaactgaggtgggagaatcactagagcccaggagtctgaggctgcagtgagccatgattgtgccactgtactccagcctgagcaacagagtgagaccctggcttttaaataaaaataaataaataaataaataaataaataaataaataataggatgaaaataggaagctaacattaatattttagatggcttcagagtagaataatagccaggaaagggtgttgagtcagattgttaatccttatccaaagtgctgggcagaagctatccacctctgaagtcagcatcttctggagcatacctaagaatctcagtttaagatttccagtggaatgggtgtccaagagtcaggaggaaatattgtatgtcttttgagctaggaaataatttacctaaggatccatactttggaatattactgctgggtcagttgttaagtatctgataaggtccatttcctcgaggatcaaaggcagttaatctcaggtcttttccagaacactaaatctcttgttgtgaatcacaggtgagttgtttggaaatgaagatcatatctgttgatgataaagctgaaggtattctatgacgcccttgcaattcttaatcatatttcatcataattgtaataggaccaaatcaaagattggtggtgaaattttcaaatacatggacaggccatttttctttttctttttcttttttttagagacagggtgttgctatgttgcccaggctggagtgcagtagctcttcacaggtgcaatcatagtgcactacagcctcaaactcttggcttcaagcaatctcccacctcagcctcctgaatagctaagattatagacgcgagccaccgcacccagcttggcctcgtattaattcataaggaggtaacctgtgggtcccagaaggggctgatcttatacacactgaagctagaggtaagattttattttatttttattttttcaatttgagagcaggtactgtttattaactgaccagcttagaaaaataatcatggaagacaccttagttcattcttctaataagcctgttgatctggtcctccctgttgccagcatctccaccttctacaaaatgggtggcctttttcttcattccacctcgtggataagataatttgaagggccacaggaagttatttgtttttttgcagacttttccaacagtatagatcccttgaatcagatcctccatgcagataatgccatatttaccaagagatcaagcaatcaaagcgttgtctgtcaaagcaattcttttcttattgattttgccataaccacgcttgtagcctagttcatttactgacttcagatttgggtacccgcatgcaatatatggctctacaatcctcagcatgttaactgaagccctgttaagcttcacaaagtttccattgaagatttgacaaaggcgaagaagctgcaacaccttttggacctttgggttcacaccattgatacctctgatcccgatgacaaatgccaatttgggttctgcacgtacatagaagttgccagcttttcttgcccccctcactattcaaatttcagttctgtacatctgcctattcgctttttcacagataagtttcctccttgccttttgaagcatcttttgggcaaacttatttctcaggcacttgatctttagctctgcaaaattccttttctttttctgaagggtttctggcacagcaggaaccttcttcttcttctctttgacaccctctatggttccagctggaaaagagaagattttagaccattggagtttaagggagctttgttcatcttagctgtcaaatttcattagttctctctgtttttcctgaggaccgtggatgataaggacagtaaagtttttgggtaaaaggcaaactttacagagttccttaataacaataccagtaaaatgagtgtcgttgtcattattagagaaataagtggaaattccaggttggaaaacaaaatcaagcagaaatttttgccacagttataacagccataactctccggcaaggaaaagctttaatccatcttgaaggagaaaaaaaaaaaccaaaataatcagagcatatttatagtccattgcaggaggcatttgtataaagtgattggaggtctgaagggccctcgagatcttagttcttgttcatatcccacctttgcatttgctaggatcgcattgttggcagtaggacaagtttttaaaatatccttggaaatacctctaaaattacccccaccaatgttgatttagtatagttgccaatttgtccctgctattatgagtaatatcgcaaagcatttttctgaaattcccctttaaatcattcgccattatgtgtcagctatcctgacagcatcaaatagtctcttttgccatgaacatccaaacttttcccacttccagattttttcacttttctacttttgggaatcaggggtttgtctctgacagttaaaaatggcctctttgaatcctccaagacccctcttttctgtgtgatttgcaggagcattgatcttggtgacgctactggggttagcatggtggccagctaaagtattactagaaattctgggtctttgtgcattgaatggtggtctacttttattacagctatttcctcaggtaacagcaaagcagctgaaagttttatttttaatctgtcatccaatttttgcagatttttgtggaggttagaaaactcttcatttccaaagcattcccaattcacagactccccaaaagtgcatctgccattagtgtatatgcttactcttttacactttagcagccagcagacccagatgcaggcaattatttcagtcattggagctgatttaatttctggagtggtctctactttaagggtgaatttaggtctgtgatagcattttaaaatatgaacaaataaaatcaggtcagggctatccaagggaatttctaacaagacgttttaagagcatgtgaaaaacactgagagttaacagaataatttggtgaaaaaaataacctctagtaatctgggtgcaaaacaactgagtcattttacatgacagagccaaatcctaattttactcctttggtacataattttctaatagattataataaaggctctattaatattcctttatttgtatgcatttaggcatacacagatatataaatatacaaataggtaacaaattaaagctttcttttttaagttgtaataaaaacacttaaaatga

SEQ ID NO: 29 - DNA, 30140 bp, Artificial Sequence
  Description of Artificial Sequence; note = synthetic construct
atgaccagttgcatccagttgtccgtggtctggttctccaaaatgtccacaagaatcttcagaagaggagaagattcgtgtggacattttggagaaccaggttatagacgtctccagtcagctggccggagtctgctacagacctgactttggaagtccctccctggtctgggccaaaaggcagggaaatgggtgactgggagtggggtgtgggactcacaaaatctgggtcatagcacagtctgaccagctccatgtggttatccataacctggttctccaaaatgtccacacgaatcttctcctcttctgtctccccacctgccacccacaaaacacagactcaatctcagcatcacagcccagccaaggaggaggtcaccttccccccgcacattgcagccacccacactcacacaggttgtgcttgcgggcaatgtagcgcaggatggcattgctctgggtgatcttgtgagccccatcaatcaagtagggcagctggatagatgggaaaccggggaagctcagttgggccaccaggctccccagcatccccttcccatatgcaagggcagagaggagaccagggcctgctcatggcaggcctgagatagaaatactgtcacactaataaatgagctgggacacagaacatcatgcaagggctgtgtagacagccaagagtgagaggaattgggcagaagactcctgatcttaacacctctaaggtggggagaggagatgcagtcagagacacgtcgcactttcccccaaaaccgccccttcccctgcacctacattgggaaagtccaggcccagcttgaatttttcattcagccactggcttctgtcatagtcaggagctgtggtggggaagaagtgaaaacaaacctccccagagcccagccctccttcccggggaccctcccaaggagccagtggcaagatccctgagacaggtggatctagaatctgaccactcagtcccacatcccagggttcagggagaaagaatcccactgagggctctggaccccacaacggctaaggcttccagggtttagggaagctctgagggacacccaacccaattagacagggtgtccagcatcccgccccagcattctgccttgcaaggccagagggctcactcacgggagctcagggaagggacagcctgcagctccagcaggggaggcctgcagaggcagccacaggtggccaccatgggttgcttggtgccaactcagcgtgagtgggcagggcccggacacgagggtgccattaccgtcccccagcgtgtacttcttttccacatagcttgagtctgtgtattccaggagcaagcggatggcgtgggccagctgggagagatggcccgcggctcgggtcagagatggagggtccctgacttggtgactttctctgcaccaggccagcccctagtctatactgcacgtacccgtcgcctcctctacccgcccccacccccaaacacacacacagcgtcatgcacaggcccttctgaggcgggcagccctgaagaggaacggtccctagagccagtcctgcagctactccacacatccccgcctccgccccctgtcccacccaccagaggaccctcgcttaccccacggatgtcccagtaccccagagtcatgggcatggtgctggttggtgcggattctgcagacaggcatcagcggagcggggctcagactttgagaggcccaggaggggacaggcggggccttgctgcgaccccgcccctcggcccgccacaaccccgaaacacgccattctgtaggcccccgagcgagcgcccggggctggaatcaataagccccctcccgcctcccagtccccagggttccgggagcgaggtcagggcgcccagaagcccaaagcggggctcggcctcgccagcttccccaacccgccctgaggctccatgagtccatccagcccttgctaggtccacagcgacttggctgtgcgcttgagacaccagccagatcctaacggagcaaagctcttctcccttctcctccctgcccgcagaatccctcagccttctctccgctgccgagttcccaagggctctgggagactccggctgcaggggtcagactaaaaagtggtggtcccaacctgggaatttaattcagcccctgtcactgtaagagcaggacttcctctgatccgaaagctactccgagggcttagtctcccctctagccccgcctacacaggaacagtgtcagtggtataggaaggacccccaggaaaagggccagagtaaaggaaatgtggtctgtgttttctgttaggggcctttggatactgagtccttcggtcatctggctaagtactatgtaaattagccacttcgtattttggcacaatttatgaatcgaaatccaaggatcagatccagcaagttggtgaggtatctgaggtgccccctagaaatgtccagccagtccactaggtgaacctcacagagaccggaacagcaacagcaacagtgagtgcagtggccacagagagcaggcgagggaggatggaagaacttcccggaccaaactgagggtccggctgctatttctctaggcccaataacgagatgcagatcaaatggggaggaagagaggttttatttctgcaaccagttacagggagaaggcctggaaattactaccagaccaactcaaaattaaagttttccagagcatatataccttctaaggtatatgtctacaagtaagtgtgcattcatctaaagacataagtgattaacttctttaatctgtaactgaggtctgggtcctgaggaccttactctggagcctcagtctatttacttaatctaaatgggtccaggtgcgggggatgattgcccttatcttgtctcctgctaaatcatggaggtttggggagttccttcagatgcccagtaaacttgtttgtggaggcctggggagtttcttcagactcacaataaaacttgtttaattctaaataggtcctgttaagaattccttcgttgttttgtcatgctttaaggcccaggaaaggcctaagcaaaactcttggtgggcttttgttacatccagcctctgtataaaggtactggcttttttcagcttttaatatttaatttcatcactcagtcagtactgaaacagttgttatggaggcctgtgttagtgacacctggtctgccacagaacctgtccctcagagaagccagaaccctgtcctgctgaggagggaggtcagaggatggtggccagagaggagcctgagggctccccacaggatcacaggcagggactctcccctctccagatccaccatgccggacctggacctggctcctgacccatgtgtgttcttcagtatgagacggtggctccagtggcctttgaagtcacaccgtgatatgtgacccatggtacaacctccgcaagaacaatgtccaacctgccaactttcttctttcaaggtagaaggaagactttcaaaagagttgtgcaatggattagcctggggttgactgctttttttaaaggatattacaaataataatggatatatggaaatagatgatagacctttaatgagaaatcattttgcaatgtaaaccaggctgttgtgctgcaaaaaaagtagttttttgttttttgtttttgtttttgtttttttgtaaattagctaaaacattgttaggactccagaggatgaacccagtatatcaaaaaagtttcaaaccacctggataatgtgtggtttggggttgagtaaacaggtagacttcatcacatccctattaggccacagaaatcaggtggtcagcaggtggctgctaagttggggcacttggacactgtgtcactgaggctggcaaggcacagtccttgttgcaaacactccccacccatccagacctgggcagtactcccagctcagatacctggcaggtatccatgtttaggaagcagttttggcctcacagtgaattctcacatagattttcctgcattgaggcaatagcaccagcacagacacaagtgcctggccctaggaatcattatatttctcattatgcattaagagtttatgattttgagttaatgggtcttgagctgtgaaagataagatgtctagatccaattgataaaaacgattttttaagaaagctaacactcatgagaataataattttgaaacacatagaaccagtactgttacattagtattgtttgaaatacttgttccttggttccataaagaaatagcacttgaacataaatttaatttcctcagcaaggctatcggggaaactgcccccagtatttcaacataggttctattttccataagtgtcagctggctgagaaataaagagaaagagtacaaagagaggaattttacagctgggccgccaggggtgacatcacatatcagtaggaccgtgatgccggcccgagcctcaaaaccagcaagcttttattaaggattgcaaaaggggagggggtgtacgaacggggggtaggtacaaagatcacatgctttaaggggcaaaaaacagaacagagatcacatgcttctgaggaaacaggacaagggcaaaagtagaactcctgataagggtctatgttcagcagtgcacgtattgtcttgataaacatcttaaacaacagaacacagggttcaagagcagagaaccagtctgaccacagatttgccagggccaggtttttccccaccctagtaagcctgagggtactgcaggagaccagggtgtacttcagtccttgtctcaactgcataggacagacattcccagagcggccgtttatagacctccccccaggaatgcattcctttcccagagtattaatagtaatattccttgctaggaaaagaatttagcaatatcttccctacttccatgtccatttataggctccctgcaagaagaaaaatatggctctattttgcctgaccctgcacgcagtcagaccttatggttgtcttctcttgttgcctaaaaatcactgttattctgttcttcttcaaggtgcactgatttcatattgtgcaaacacacatgttttacaatcaatttgtacagttaacacaattatcacagtggtcctgaggtgacatatatcctcagcttatgaagataacaggattaagagattaaagtaagacaggcgtaagaaatgtaaaaagtaataatttgggaactgataaatgtccatattaaaatgaaatatttgcaatttatgttcctctgccgcggctccagtcagtccctccattcggggtccctgacttcccgcaacatctctccctttctttttatataaatgtgccatggtgatgaaggcttattcgttctctcggttttgacacaggattctttggctggtccggcacacagtatccagattccttctcgagctccaaccaacattacacttttcctggagtcaaaacgggagtcaacacaagtgtataaatgacaatgcattggacagtttgattgtttgtccaaattttgatatttgccactaacagcatgtaaggaagcttaacacaactctgtataggaatagtcaggttggaggtaaacaaagcagaatgcttgaatctatgttgatactgagggagaggagcggcggtggcaatgcccgatgttctccaccataaagaagcaaaccgaggtgcctggggatgctgaagaagtagaggggcatacctgggtcgataagaattatcgtaatgccaattggagtcccgtaaaggaggatcggcatcaaaaagaggaaaaaggttcaaaggggatttatcatggggttcagaatcacagatactggcagtccagacatgacaatagccaatttcgaaagttctgggtgttctgggctcagaatggggaatatcatacaaggccttgggggtggagggtaatgtccttatcttcccattttaaggtaaagaatgagccgaaccacctatgcaaagtaggatgatgatcctcgtcctcccaataagaaataaaataagtagcctccaggaattcccctcccccagaggagcaagtgttttttaaatagccctttggtgcccagtctattactaaagcatatgagtcattttttaatactactgcatgtgagttaacacaatctccccaaattaaagttttagatgggccctcaaaatttttaggacatagttttcctacaggtttatattgaaagtatggggtatctcctattactccccttttcatttgtcttaaaggacaaagggagaggccggagaccaaatgtccccattttcctgtagctaatctctccagaagataagcagcccagacttgagtttctagatggatacaacgagttgcatgtccgaggcacagaggagggtatttataacccatagtaacattaaatgcagtgccttcttctcctggttgagcagggcaatggtcatctatagctccaggcatctacacactatcgttagtgtagatttccgcaggagcattcatccaggtgagaggtcgaataagtggaggaaaaggcacataagcccaataagaataattttgtgtagcaggcaaatcagtgtgaggggaaactggtgagacagaaagtataaggagtagaattattaaataaaacctattgtaagcgagatccagtgctgaaggaggaagagaagaacagagggatgttattttcaggctaatagaaatggtgagattttcaggtttgtaaggagaaaaagaaaggtaattaggagaagtgggattagttagaggggtctctgttgccattagggaggattgaatgagatccattttgatttggtgtgccagtttttgaggagttggcacagatctcaccaggtatgagggcagtctctgacgtggacatctcttccctgtgtttttcattgtcagtattcacacgaagttaaagtcttctagtgggcacccagacaggggattgatgatctcctggtgaaacacaagcataccctcttccccacattataattgttccaggttcccaggtatttgtctgggagtttttccataacactggcttgctttcatttagggagaattttttgcctgtataatggtgttcggctgcagtcagagtattgtctttaggaacatttagaaagtttaaagtaaacaatgctaaatggaattgggagtggggagtggttaaattatgcttttaaaccataaaggaatttagtaattccttcatgttttggaccgagactgagtccaggaacagtccccacgttttccattatatgttgactgggagcacgagaagagttatgtggaatattaatttcagccccccattgttccagcaaatatctaccccaaagattaatggggattggcatgatataaggctgaattgttcccttttgaccatcaggcccagtgcaaggcaagataaatgtgctttggtgaacttcatcagcttttccaacccttactagtcccatgttagtgggatgtttaagccaggaagaaggccataaattagaggaaataatagaaacatcagccccagtatctactaggccctcaaacttttttcctttaatatgtatggtgcaggtgggctgttgtttagaaattactttaatccaataagcggctttttcaccgccagagcccatcccagggccccctgtcttatctcctttgtttaaaacaatattaggtagtaaaagtagttgagcaattgactcactggccggaatggaaacaggaaccttggcagacacgattattttaatctcatcagaggaatcagaattaatgagaccagtatgaactatgatacctttagcagaggtggatgccctacctaacaccaggcccaccaaaccttgaggtaaagggccagtggcccccgtggggacaattaaaggcaaagaattcggtagtaaatttagaggagtggtactacagagattgaccgccctgccccctactgtggaggtagacaagcattgtactgagacagaagaagcgactgggacccatctgggttggctgtaggtaaatttgcttgtgctgggggctgtattgggaccgcttgaagcagaaacacaatattggtctgagtctgaggtgtcccatttgatactggggcctgggactggccctgcttcccatttccctggttctgtggcaagggattgccatctatatcagacttagagtggcaagtacttgcccagtgtttacctttacgacaatgagggcaaacagtagcaggagcatttggccatgtttgttgagctggcttggccgcttttaacagtgcaatttttttgggtatgatgaagttgaccacaattatagcaggctccaagaaaagaattcagtcaagccagtttgattgccatccttcatggcccgtgcccacagaatagctttgtgggtctctgatccaatgctttcacaagctttaatatatgcaggcacacctcgtgatcaggtaaattttgtcattagacagaacgcatgaccactttacactcatggttcgcattctcaaaagctaacatatgaaggagaataccttgagcgtaatcatcagagacagattttgaacagcatcttgtaatttagccaaaaaatcaggatgtaattcactgcgaccctgtttaacagtagtaaaagaaacaggagcttggcctggggcacctagtttatcccaaactctcatacacaactttgttacttgttccgtggtgagagcgtcaaagtctaattgggcggtagtgtcagagtgattatcagagcctgtgagctgagcctgagtaattagaatgccatcaacccaatttagctgagcctgcagacgggcctcctctgaccaccaggtacagaattgtaaatgctgagatggagttagaacagctttcgccaaaaggtcccattctgaaggaagcaaaatgacctcagtacaaagagtctgtaatgccattttaacatatggagaagcaggaccatactgagcacaagcatccttgaattcttttgaaaaggtaagattgagcagtgcatatcaacgcatttgtaccccttgagcattgggaggttccagcgtgaccggataagcccacacctctaatccacttgtttctttgttctggcgtaataagcattgcatggaagtttcaagagcaggcacatgagatggagttggcatagaagtgacagtaaaagtatgtgtagataagggaaactgaggttcaggaggtcagaccagtatgagagtgtgagaaaggggcattgggggagcagaagagacagaagcatactggtgattattgggaaatcctgtgcaaccagagtggcaggggcgtccatgtgtgaagaagggtacagagaagcaggttgagtgaatggcaaagtgactggttgagggaccgaaatgacaggagggtgaggggctgcggtgggggagggcctgcagaattacaggtaaattgtagtttggtctggagccattagatggctctggaggcaaagcctgcaaagatttgaaaagggaagagttagcatagatatggtgcagagctgtccgagtcagggccatgggagctacaagtatcaggtcttcatgaaaagaaataagaccatcgggggtgacattaagccaaagtcaccggagttagatattgaatcctcaatatcgttaggtgggggaggagtaggcgaagggagaggctgagcagataatgaagcccgagtgggagaggaaagctgtggaagaggtagaaggtcgccagattcagaaaactgtggtaactgcagggggtcacgggattggtatgtcgttaggatggcacataccaaggcccaatcaccccaaacagtgaagggaacataatttcctgatgggatcagttcctggaattttgcaccaacgcgatcccatagttccacatctaattttcccttttcaggaaaccaaggacagtgttcttccaacaccctgaatagggtgaccatattttccatgggcacccaaactccaccctgttttaacaggagtttaatatagcagagataagcataatatttagactccatgtgacccatagttacccgagacaatacatagaaaattcaccaatcatcagggagccgaacaagcgtttctgaggactggactgatgatcgtttctccacacctaccaaagggaatcaggttcccacatgcacttaggaaaaagaaaaccatgttggtgcaccagatattgggggaacctgcccccagtatttcaatgtaggttctttctactttccataagtgtcggctggctgagaaataaagagaaagagtacaaagagaggaattttacagctgggctgctggaggtgacatcacatatcagtgggaccgtaatgcccgcccgagcctcaaaaccagcaagtttttattaaggattgcaaaaggggagggggtgtacgaacagggggtaggtacgaagatcacatgctttaaagggcaaaaagcagaacaaagatcacatgcttctgaggaaacaggacaagggcaaaatagaactcctgataagggtctatgttcagcggtgcatgtattgtcttgataaacatcttaaacaacagaacacagggttcaagagcagagaaccagtctgaccacaaatttgccagggccaggtttttccccaccctagtaagcctgagggtactgcaggagaccagggtgtatttcagtccttatctcaactgcataggacagacattcattcccaaagcgaccatttatagacctccccccaggaatgctttcctttcccagagtattaatattaatattccttgctaggaaaagaatttagcaatatcttccctacttgcatgtccatttataggctctctgcaagaagaaaaatatggctctattttgcctgaccctgcggacagtcagaccttatggttgtcttctcttgttccctaaatatcgctgttattctgttctttttcaaggtgcactgatttcatattgttcaaacacacatgttttacaatcaatttgtacagttaacacaattatcacagtggtcctgaggtgacctatatcctcagcttaagaagataacaggattaagagattaaagtaaggcaggcataagaaattataaaagtattaatttgggaaccgataaatgtccatattaaaatgaaatatttgcaatttatgttcctctgctgcagctccagccggtccctccattcggggtccctgacttcctgcaacaaaggccatttttactttctgcagaaagggtacactcgccagcagttttaccacgagagtacactgaacaaaggagaaagggtcatttataacttgatgtgtccaccctactgctgtgtccagtttccactggctggaacgggacctcacattctgtatttgtcctgattggctagcaacttagaactttttaaaagaggcagaggcagaggagaacaaaggaagggggaactcacttgtggaacgctgagaaaggtaaaaacaccttcaaataaggaagaggaacaggctatgacctcatgcttgtttggatcagtataagcatgccagggcaagtatttaggctaaattgtgggagttaagaacataaagtacattaatttctttattacagctagcagatatttcagaatatcagcaccggtctttgaataaattttgcttctaagagaagttacgacttattcttaattagatggggagaaaagtctctttgaagaggaacctctactttactttttacagtattaatatatttggtcattcctttattgaaacaacaattgtcgagatcctactacattccaggcactatgaataaaatgatgcttcaaggacgctgtatttgcccatctccaactacatatcaggtgggaacttcaggggagagattttgaaaatcaggacacatcttcgtgttttggcacactgagaggcagtggagtgggagaggggttgagtgcaggagagagaagtagtgcctgctgttgcaaggactctgaggagccaggaagaaggccaggccccaggtggcttcttcagaagcaggagtagagctctgcggaaggcgggaaggactggactagcggctctccaaagtagctgccatagtggccacaaggatcacaccagcggggaaagtgggtctgtggacggggatcaggagcagcagggatgagggaagctgcaagtgttcagaaggaaggggttgtagccagttgtcaccacacagaaactcagtcccgacagggcctggacttctgcatttgtatgtgctgtctgaactgcaaaatgtggaaaagttctaatttttaaaacaatgctgtgccagacagtcaaaatgcactgggtcccactttaccaagggcctgtaatgggacctctggactggatttcataattccttctgtctcttctgacaacattcttacattctcaaggcaaattaacagaaaatctacctaacatgttgtcagaattttaaaaactaatctattagaaacaaatactgacagacaaaataaaatgtatcctaattcatggaaatgtattaaagtctcttaaatattgtttgggtaaacttaggaaatctgaaatataaagaatcttttcttcaatctgaaaagaagaaaataaaaatcaccatttgaagatgctaccaaatttgaattgagaaaacatttttccaacatgcctgtgataccaagaataaggcaaacaaactagaatttcaagaaagtagaacaggcacaaagttagagatggaaacagaagtggaagcaataagttaccttaagagaaatattgaaactttaaccaatttgcttgttatatgtaatagtttattctgttaaaagttaggtaactacacgcctgtaatccccacactttgggaggccaaggcgggcggatcacgaggtcaggagatcaagaccatcctggctaacatggtgaaaccctgtctccactgacaatacaaaaaattagctgggcgtggtggcagacacctgtagtcccagctacttgggaggctgaggccggagaatggcatgaaccagggaggtggagcttgcagtgagccgagatagtgccactggactccagcctgggcgaaagagcgagactccgtctcaaaaacaaaaacaaaacaaaacaaaaaaaacttaggtaattaacacagcattagtacaacaaaaacaaaaaatataattataagttaatacaaatcagaactaagaaagaagatgtagtacaagtgattttttaaattggggaaacatttttctaaactctatgttaattgatgttaccaccaagtggaagaaacacaaacaggccaaaacatgttaagagataaagattctgaatgaagtacaaaaagcgtgataaagaaaaataacaacacaaaagggcattttatcctcagcattaggctggatatgttttcaaaaattttgaaaatggcatggatgacacagattgtcataaactacagacatgacctaacatgggaaggacaacacaggcaaggatgattataaatcactgtcacttatcagcctgaatccaaacatcaggaatgctacctcagagaaaagaaaatagtgtttttcttataatggtgttgtgctcattcggagcttgctgggggagacagagacagaaaggcgggggcaggagaaggaggggcagagggtactctggggggcagaaagcccatgtggaaacattagtatctagggtaatcagagtcgtttctaatctttctacaatttctcaaactctgacgctagatcaatataaaaaggtaaacaagaaaattacatatacatatacccatattattaggtattaatgatacttatattattaacttaaataaatgttccatcaaaaaggaattaactacatgagctacattatgaatctttttattattattaatatttttaatgctagtatccatccagcacaccactggatgtgattcaagtgaaggtgattttgcccctcaggggacatgtaggtctgtctggagactttgttaattgtaaacaccaggaagcgcccgtggtattagtgggtagaggatgggtaggatgctgagcaccctgcgatgctcaggacagcgccccacaacaaagaattctgccctccaaatctctgtacggccgaccttaagaaaccctagtctgtgatccaggacagggtcgaaagctgcagtgggagaaacaaggtcctcgaaattctggctggcaggaagcaggccagaggacccacttccctggacaccagctggcagaagggaggacctagagacaatctcagctggtgcagcccatgcaggcatcaagctgcacatcaggaatcagagaaggagaattgggcaggcaaacggtctactaggtggacaggctgggcagcctggtcttccagggtacagggtagggagtggtgggactctctctgccagtcagacagcaggcctcccctgcagctctcagcacattgctccagagccaggcagcctgagcccagcaccatcgggtaattcttctagagctgctggggcaaagctaggcaggatgaactgaacttattttcaaaaggtatccaccctgactgagaagggtgtgtagaaagttgctacgagtcactgccaacttgtaatggcttgactagaccctccctgcatttaacacccccagaggggtggccaaaagtcaaatatataataagcacctgatgactgccacagacatggatatgagtgatgctagtcaccagacgcagcccccttcccactgctcacccacccttccaagcctgccagagaccctcacccaaaatgaattagcaaaatgtgcaagcagatgttttcatctctggagactttaaaacaatgatcatcaacacggagatttaccaggaaagataatatcttccctcaaaatctccttggagtttactgtgaggcatctcagacagtgctttccatatagaaaaaaacccaggtaatagatatgctgcagcgttttatcttgtgtttgattttataaaacttacttattgatgatggtgacccatgtgtattttttgttgctacgtgtactagtcaagggtcttcaaaatttgaagtgacgccactaagataagcctgtaggaaatttgtgtgtttatttacaaatttatgtatctcttgtgggaactggctcacataattttggagtgaagtcccacgatcagctggctgcaggacagagaagcgggaaaggccagcggtatcattcagtgtgagtctaaaggcctcagaacaaaggtgggggtgggaggcgatggtgttagtccctgatcaagtctgaaggccagagaaccaggagtgtggaggtcccaggataggtggagatggatgtcccagtcagagaggtcaaattcacccttcctctgttttttcttctctctagaccttgacagtttgttggaagcccccttcattgctgagagaaatcttggctcagtcttctcattgaaaggcaaatctcttctggtaacaccctcacaaacacaatgattttacctgccatctgagcatcccttagcccagtcaagttgacacagaaaactaaccatcacattatgccacaggaaaccaagaacaaaatcacaattaattacattgtgcctatgggagaaaaagccttgactgccacaagatcattctttaatctttctcctggagatgactaagccaccggtagagaatgggtaaacagagagttttatacaggagattcacgaacaagtagattcatagactgtgattcattggcagaaacatgcacaacctataaacaaaaaaggtgaaggtagaaaatttaagggtattctttttgttgttgttgttgttgttgttgaggtggactctcactctgtcgcccaagctggagtgcagtgtgcaatggcacgatcttggctcagtgcaacttctgcctcccaggttcaagcgattcttgtgcctcggcctcctgaatagctgggattacagttgtccaccaccacgcctggctaatttttgtatttttagtagagacagggtttcaccacattggctaggctggtctcaaactcctgacctcaagtgatccaccggcttcagcctcccaaagtgctgggattacaggcatgagccaccatgcccagccagaatatttgaaggtgttcttattaaaaaaaaaatcgtaagatgaaattgcgatctcctaagtgcctaaaatagtgcagaaaggtccgtgtactgtaccttgctggtggcaccataaattaagttattgtctctggagagcaatcaaattatctgttataccattaaaccagtctcaccctttaccttatagtaatcacctttggggatctactcaaagtaaccaaaaatgggaaggaattagtcaaaacatgatcaccctgctctaaaaacaaataaataagttagaaaaatccacatacgccatagcataagaatgactaggcaaactatagttaatccacaggaactaatgtgagatagccatcaatattagaagtaggcatgcatatagactgccaacatatacagttttttttttttttttgagacagagtcttgttctattgcccagactggagaggagtggcactatctcggctcactgaaggctccacctcccaagttcaagcgattgtccggcctcagcctcctgagtagctgggactacaggcaagagccaccacacccggctaatttttgtatttttactagggaagtggtttcaccatgttagccaggatggtctcgctctcctgacctcgtgatctgcctgccttggcctcccaaagtgctgggattacaggcgtcagccaccaagcccagcccatacacagattttttaagttaaatgttaggccaggcgcagtggctcacacctttaatcccagcactctgaggggtagaggtgggcagataacttgaggcccggagttcgagaccagcctggccatcatggcaaaacttcatctctaccaaaaatacaaaaattagccaggtgtggtggtgtatgccagtagtcccagttactcaggaggctgaggcacgagaatcgcttgaacccaggaggcagaggttgcagtgagctgagactgcgccactgtgctccagcctgggcaacagagtgagactctgtctcaaaaaaagaaagagaagtaaaatgttaaagagaaaaaatggtacaatctaagagtatacatgcacaccagtctcacctatccaaagaagtaggcatgaataaaaatctaaagggaatttgaaatcacacctttaagacgaacctataatttgcttaataactattaagtgagcacctactatgctcaaggactgtgctaggtgatatgcaaacacccgttacatcagtgaacaagaagatacaaaaacacatgccttcatgacattgacatattatgagggaaagcaaaccaacacaagcacacaaataatgtaagacatatgtgcatgcttttcattgaagtgaatacacatacagtgtacaggccttaagtgtgctactctgtgggcttttccacttggatactcttctggaatcacctcccagataaagagagaaaacttacagcactgcaccaggagccttcaataggcaaccttccagttctcaccaaggtcaccaatcatctgacctctgataccatggactcatttccctcccctgattttcgtgtaagtggaatgagattgtgtgtcctttctcctgtctggatccttttactcaacacatctgtgagacccatccatgtttttgcacatagtgttagcacatttattttcattatagcagagtaagtattccattatacgaatgatggaattgtgttgataaaccacagttcatttgttcattcttctattcatggaatttggattgttttcagttattggctatttaaaataaagctgcttttggcatcattatacctggttcttggaatccattaggactcattcctgtagggtaggacttctgggtcctgggtagatgtatgtttagctctagttgacagtgccagtattccaaagtggctttcccaccagcaatgcatgacagttccagttactccataaatcaccaacaattgtcggtcctttcaatatcagccattctagtggggatacagtaggatcttaggggtttttaactttctgctgtctaatgatgttgagcaccttctcatgagcttagtggccatttggatattcacttttgggaaatgcttgttgaaattctgactcattttttaatgagttgtattttcggatatatagtttttggagaagagacctttgttgatttgtaacaagtactccctgctgagccttgcgtgaatacctgacccacagactcaacaactatttctggtatcacagcttaggaactgctaagctatgagaatcttcctctgaagcagttcactctccagtgagattttaaattacttcaagaagatgcaatgcagataaagatatgggattgctgtcagggtggaggaagcctatgcttagaaagttcctgcaatgatgtgagtcaggaagagatcaacctgtagatccccacacggcctttggaattatagacctcagaatgataaacagaaaaagcttaagtaaaaaagcaaaagatatggtcaggcgcagtagttcgcgcatgtaattccagaactttgggaggcctaggtgggtggatcacgaggtcaggagtttgagaccagcctggccaacacggtgaaaacctgtctctaaaacaaaaatacaaaaattagctgggcacagtggtgtgcgcctatagttccacctacttgggaggctgaggaaggagaatcgtttgaacctgggaaggggaggttgcagtgagacaagatcacaccactgcactccagcctgggtgacagagtaatactccctctcaaaataataataataatttttaaaaagcaaaagatgctgtcagaactggcatccacctactcaccagaaatgctcaattctagaaaagagcgagtaaatctattagaggtctgaaggttttcatcatagaattctatccccagctgatatggtttggctgtgtcaccacccacatcttaccttgaattgtaataatccccaggtatcaagggtgggggccgggtgcagaaaactgaatcgtgggggcagtttcccccatactgttctcatggtagtgaataagtctcacaagatctgatggttttataagtgggagttcccctgcacacgccctcttccctgctgccatgtaagacattactttggtcctcattcgccttctgccatggttatgaggcctcccaggccctgtggaactgtgagacctttaaacctctttcctttatcaattacccagtctcgggtatgtctctattagcagcgtgagaacagactaaatcctcagccagagtattcagtaagcatgtaaaggcactttcagttatgcaaggactaaagacatttctctcacaggtacccaggaaagagaggaagtaaacccagagagaacgtcctgatgacagctgtgcagcaagcctggagagggtacctggaagccatctgagaaaaacaatagagattgggtcctggagacctaacagaagtgatgtagtcatatagtggagaaggtaaaagggaccctggttctctctgatggagattccacccccttggggtcctattcaatgagacacaagctctgtgcaattatggaaagtactcaatacggctggatgaatgaatgggacatccaggccaagaaagagacatcctttccacagtgcctcatggggcttcctgctctgagtctctcaggaatgggagcaagaattctcaagcgccttactatgcccagcagaatcctttagaacccagccactgccaggatcaccagccagtcccttggaagaggcagtgacagacagacagacagtctgggtagatggccaggagcaaataagacaagacacacagcaaatgtggttgaggctttacttccatcaggtaggcctaaggcagggaccatgctgcaggagcagggcctgtaaaccagtcaatgctgctccttcatgcaacacggggacagctcggggctgagctccacaggcgaggggccaggcaggccctttaaagcagacacaaccactaacaggaaggaaggtcaggctggctttagtgcagggaagggtaatgatgggaggggatgttcacgaaggatagtgggtagctgaggcttcaaagggcctgcatgggacaggggtttagggggagtgaagagggacaatgaagtcttgggagtaaagatgggaataaacaggagaaaggaacgaggaggcaggtgctgggatgcagagtccagctgcgctgcacagcctgggcagcaggctgagtatgggctcctcactcccacctcctggccttcaaggccctacttgttgccccagacagccatctttgagaacacaggtcttgggaggaagcggctggacttcatgtaggcagagatcttctccaagccctgagaggggggaaaataaggccagaactcaaggtcagctgcccatgagggctgggacaaccccactgcagacacagcaggtcagggccctggccacagcctttccatgtgggctcaagtgctgaggcatcacacagcttcttagggtccccagatttgcggagttaactcttccaacttcctttaagtaatgttgtaccacagtaacagctaacacttatgaagattctacacggtgctgggtgctctgtacccaagagaaattacaacgtaagcttagaaaacccactcgactgctctgcaaggctgaatctattatcaccatttctcatgtgggaaacagaagttcagcaacttccttacatttaaatacctagtaaggggaatagaactgcagtgtagcctggttctgtctctctccagttctggcctcttcctgacaaattctcttcttcttctgccccaccacccctcacaccaggccctgtcactccttgactgcacaaacagtctccttttccacagggaggctcttggcttgtctgggtatagagacacagaagggacctccagcagaacaaaggggctctgaacacactcacccaatgtctcagtaaaacctggatgaaagtggaatggggggtggcagtgcacaagaaaatcaaaggcgagactaggctctaaacattccccttaaagcagaaagtccaaagttctccatctaatacctgagcaaaatctcactgtgccctaacccatggcatctccagccctgctgcagatcaggctacatgatactcagatgtgctgtgcacaatcttaccaggattgaatgcccagataaggtattcaaaaacatccggctcacacctgtaatcccagcacattgggaagccgaggtgggtggatcacccgaggtcaggagttcaagaccaccctggccaacatggtgaaagcccatctctactaaaaatagaaaaattagctgggcgtggtgatagacacttgtaatcccagctactcgggaggctgaggcagagaattgtttgaacccgggaggcagaggttgcagtaagccgaggtcacgcaactgcactccagcctgggcaacagcaagacacgatctcaaaaaaaaaaaaaaaaaaaagatccaggactgcctgttctcaaactgattgaccctggcatgtcataggccatagacctcagctctcctccacccctcccttcccaccacctcttacataactccaaggccactaagcctggaccagaaccagagctgatgggaactagctgagcaagcagatttcttgatgggaaagggaaacagattctaggctcagaagcccgttggactaatgcctcaaattcccagatctgtactaaagtaggatttgtgctctgaaacaagatcagcaagggtgagggatggttccccgtgagtgaggggcaggccccgggcctggtacccaggtggtgccacacacacctcacaggctgctctgcctgcagccctctcactgcactcatttccaggatggggcagtcatctatacatctcccactgtgcctcactccctgcctcatgcagagaaggtcggtgaataggagtaaaatcagcatgctactcatctcacagggggaacacgtggaacactggagggaaaaaagaggaagaggagtggctcatatctgtaatcccagcactgagggaggctgatgtgggcagatcacctgaggtcaggagttcgagaccagcctgaccaacatggcaaaaccccatctctactaaaaatacaaaaattagccagacgtggtggtgcacacctgtaatcccaactaatcgtgcagctgaggcagtagaatcgcttgaacccaggaggcggaggttgcagtgagccaagatcactccattgaactccagcctgggcaacagagcaagactccagcacaaaaaaaaaaaaaaaaaaaaaaaaagaatagaggtggagtaaggaagtgccccagcaccgggcatttcaacatatactccttcaacagggacacccacatatgtaccttaatccagctacagccacagccctggctctcaggattccccacactggggtccaatcatgcaaagcttctggaggtagctatgaatattgcctgtgggttcagtactagacatcaatgtcaccgttgtgtcagtcctactattgccagctccatctatggtcatgaagttctctgcctcttccaaagtgtcctgggctgtgaaacccaggctaggtgaagcctaaggctttttcttctgtcacaaatgggagagccctgactgccacccatagatccagaggctcacagggagcccagcacagcctccactagctctgggaccctgagcaaaggactgaccctctcattcttagtttccttatctctaaaatgggcgagataattctgttaccttactggaatgttataaaattcaaatttcacacaataggtataatgtgctgggcacggttcgtggcatgaaaccagtactcaatacatgcgagttattctgtgtgtagctccaggactgggaaaacatctgaaaggaggtaacggaacaaggggcatcaaagagaaaggaggatgggggcatcacctcaaagcgggagatgaagtccttcagatttgggaaggcgtccaagcacttgggctcaaatatacggtggaggtcaaggacatcatagacgagaaaatctacaaaagtgatctgcagaaggccaagaatatgtgggctggaacctccataacacgtgaagcaaaacagatctcattccccatatcactcctcctttaccttgtttcctgcaaaccatggccgcttccccagaaactctgagtagagctttagcttttcagggagttcctccaagtactttggcttcagtttctcctgaggcagaaaacagctgtcaccaccctcggacaaaagctccctgcacagacgtggcctccccggggctgtggacggcccaggctggccatgggcaagcagccatcttccccaagctggaactggggcagtgcccaggcttcaattggatacatcagcatttattagactctgactgagtgccagaccctatctaaactgatcagaaggcagagaggaatgagatgctgtccctgaatctgtcctcactgacctcccaatagcctctgggtcagccccaggagcgcccaagcacctggcagcctctggatccagaccagcagaatcaaggatgcaaagaactaagggagctcagggaaagtagaaagttccttctaatggggtggcagaagaaaacaatgaacattgactagtttccagggagatgagaagttcaagttgaaaggaaggagggagaggaataaaggccctctgactgcctctgagctgtctgttatgaaaatgcgcagatccctgtgcatgtcaatgacagcactcagaaaactgttgaggagaagatgcaattcgagggtgccattacattcctgagtcttctacttgagtctgcatattctaggagcaggcaggtgatgtgaactggctgacagaaatgacccaggacagaagtcagaggccagagctgatgaaggagattctgaacacaaactttaccatacaggaactgaatttctaccctgggatgcctggatcgtgtcctaaggttggtgggaattgcctctgcttttgttgacatgacattggagtcgtgagaataactgctgatagtatgggaaagcacttggaggatgaatggtaccagggacttaagaccagtgtgtgcaggaatgcaagagtcctggccccaaatccaaactctgtcagatgcagctcactggggacactcacaaattctggattgtagcagatcatgcccagctgcatatggttgtccatggtctggttctccaaaatgtccacacgaatcttctcctcttctgtctccccacctgccacccacaaaacacagactcaatctcagcatcacagcccagccaaggaggaggccaccttccccccacacattgcagccacccacactcacacaggttgtgcttgcgggcaatgtagcacaagatggcgttgctctgggtgatcttgtgagccccatcaatcaagtagggcagctggatggatgggaaaccggcgaagctcagttgggccaccaggctccccagcatccccttcccataagcaagagcagagaggagaccgggcactcactgtgcctgctcatggcaggactgaaataagaatactgtcacatgaacgaatgcaggtggacacagaacatcatggaaggactgtttagacagccaggagtgagaggaattgggcagaagactcctgatcttaacacctctaagctggggagaggagatgcagtcagagacacgttgcactttcccccaaaaccgccccttcccctgcacctacattgggaaagtccaggcccagcttgaatttttcattcagccactggcttctgtcataatcaggagctgtggtggggaagaagaagtgaaaacaaacctccccagagcccagccctccttcccggggaccctcccacggagccagtggcaagatccctgagacaggtggatctagaatctgaccactcagtcccacatcccagggttcagggacaaagaatcccgctgagggctctggaccccacacagctaaggcttccagggtttaggcaagccctgagggacacccgtcccaattagacagggggcccagcatcccaccccagcattctgccttgcaaggccagagggctccctcaccggggctcagggaagagacagcctgcagctccagcaggggaggcctgcagaggcagccacaggtggccaccatgggttgcttggtgccaacttagcgtgagtgggcagagcccgaacacgagggtgccattaccgtcccccatcgtgtacttcttttcctcatagcttgagtctgtgtattccaggagcaggcggatggcgtgggccagctgggagagatggcccgcaggtcgcgtcagagatggagggtccctgactttgtctgcaccagggaagcccccagtttacactgcacgtacccgttgcctcctctacccgcccacacccccaacacacacacagcggcatgcacaggcccttctgaggcgggcagccctgaagaggaacggtccctagagtccgtcccgcagctgcttcgcacttccctcccctgcgccctcgtcccaccgtccagcggaccctcgctcaccccgcggatgtcccagtaccccagtatcatgggcatggtgctggttggtgcggattccgcagacaggcctaaaccgagcagggctcagagtataagagctccggagaggggacaggcggggccttgctgcgaccccgcccctcggcccgccacaacccgaaaggcgccattctggaggcccgggagagggcgcctggggctgcactcagtaagacttcctcccgcctcccaggcccgagggttccgggagcgaagtcagggcgcccagaagcccaaggcggggctcggcctcgccagcttccctacccagccctgaggctccacgagtccatccagcccttgctaggtccacagcgacttggctgtgcgcttgagacaccagccagatcctaacggagcaaagctcttctcccttctcctccctgcccgcggtgtccctcagccttctctccgctgccgagttcccaagggctctgggagactccggctgcaggggtcagactaaaaagtggtggccccaacctgggaatttaattcagcccctgtcactgtaagagcaggacttcctctgatccgaaagctactcccagggcttagtctcccctctagccccgcctacacaggaacagtgtcagtggtataggaaggacccccaggaaaagggccagagtaaaggaaatgtggtctgtgttttctgttaggggccctcgggtactgagtccttcggtcatctggctaagtactgtgtaaattagccacttcgtattttggcacaatttatgaatcgaaatccaaggatcagatccagcaagttggtgaggtatctgaggtgccccctagaaatgtccagccagtccactaggtgaacctcacagagaccggaacagcaacagcaacagtgagtgcagtggccacagagagcaggcgagggaggatggaagaacttcccggaccaaactgagggtccggctgctatttctctaggcccaataacgagatgcagatcaaatggggaggaagagaggttttatttctgcaaccggtaacagggagaaggcctagaaattaccaccagaccaactcaatattacaaagttttccagagcatatataccttctaaggtatatgtctacaagtaagtgtgcattcatctaaagacataagtgattaacttctttaatctgtaactgaggtctgggtcctgaggaccttattctggagcctcagtctatttatttaatctaaatgggtccaggtgcgggggatgattacccttatcttgtctcctgctaaatcacggaggtttggggagttccttcagacgcccaataaacttgtttgtggaggcctggggagtttcttcagactcacaataaaacttgtttaattctaaataggtcctgttaagaattccttcattgttttgtcatgctttaaggcccaggaaaggcctaagcaaaactcttggtgggcttttgttacatccagcctctgtataaaggtactggcttttttcagcttttaatatttaacttcaccactcagtcagtactgaaacagttgttatggaggcctgtgttagtgacgcctggcctgccacagaacctgtccctcagagaagccagaaccctgtcctgctgaggagggaggtcagaggatggtggccagagaggagcctgagggctccccacaggaccacaggcagggaccctcccctctccagatccaccattccggacctggacctggctcctgacccatgtgtgttcttcagtatgagacggtggctccagtggcctttgaagtcacaccgtgatatgtgacccatggtacaacctccacgagaacaatgtccaacctgccaactttcttctttcaaggtagaaggaagactttcaaaagagttgtgcaatggattagcctggggttgactgctttaaaggatattgcaaataataatggacatatggaaatagatgatagacctttaatgagaaatcattttgcaatgtaaaccaggctgttgtgctgcaaaaaaagtagtttttttgttttgttttgttttgttttgttttgttttgttttttgtaaattagctaaaacattgttaggactccagaggatgaacccagtatatcaaaaaagtttcaaaccacctggataatgtgtggtttggggttgagtaaacaggtagacttcatcacatccctattaggccacagaaatcaggtggtcagcaggtggctgctaagttggggcacttggacactgtgtcactgaggctggcaaggcacagtccttgttgcaaacactccccgcccatccagacctgggcagtactcccagctcagatacctggcaggtatccatgtttaggaagcagttttggcctcacagtgaactcttacatagattttcctgcattgaggcaatagcaccagcacagacacaagtgcctggccctaggaaacattatatttctcattatgcattaagagtttatgattctgagttaatccgtcttgagctgtgaaagataagatgtctagatccaattgaaaaaaaaaacgattttttaagaaagctaacactcatgagaataataattttgaaacacatagaaccagtactgttacattagtattgtttgaaatacttgttccttggtgccataaagaaatagcacttgaacataaatttaatttcctcagcaaggctattggggaacctgcccccaatatttcaacgtaggttctttctattttccataagtgtcagccagctgagaaataaagagaaagagtacaaagagaagaattttacagctgggccgccaggggtgacatcacatatcggtaggaccatgatgcctgcccgagcctcaaaaccagcaagtttttattaaggattgcaaaaggggtggtggtgtacgaacagagggtaggtacgaagatcacatgctttaaagggcaaaaagcagaacaaagatcacatgcttctgaggaaacaggacaaaggcaaaagtagaactcctgataagggtctatgttcagtggtgcacatattgtcttgataaacatcttaaacaacagaacacagggttcaagagcagagaaccagtctgaccacaaatttgccagggccaggttttttccccacctgagggtactgcaggagaccggggtgtatttcagtccttatctcaactgcataggacagtcattcattcccaaagcggctgtttatagacctccccccaggaatgcattcctttcccagagtattaatattaatattccctgctaggaaaagaatttagcaatatcttccctacttgcatgtccatttataggctctctgcaagaagaaaaacatggctctattttgcccgaccctgcggacagtcagaccttatggttgtcttctcttgttccctaaaaatcgctgttattctgttctttttcaaggtgcactgatttcatattgttcaaacacacatgttttacaatcaatttgtacagttaacacaattatcacagtggtcctgaggtgacctatatcctcagcttaagaagataacaggattaagagattaaagtaaggcaggcataagaaattataaaagtattaatttgggaactgataaatgtccatattaaaatgaaatatttgcaatttatgttcctctgctgcagctccagccggtccctccgttcggggtccctgacttcctgcaacaaaggccatttttactttctgcagaaagggtacactcgccagcagttttaccacgagagtacactgaacaaaggagaaagggtcatttataacttgatgtgtccaccctactgctgtgtccagtttccactggctggaacgggacctcacattctgtatttgtcctgattggctagcaacttagaactttttaaaagaggcagaggcagaggagaacaaaggaagggggaactcacttgtggaatgctgagtaaggtaaaaacaccttcaaataaggaagaggaacaggctatgacctcatgcttgtttggatcagtataagcatgcca